(19) United States
(12) Patent Application Publication
(10) Pub. No.: US 2026/0027255 A1
(43) Pub. Date: Jan. 29, 2026

(54) Cross-Linked Telechelic Polymer Compositions, Methods of Preparation Thereof, and Methods of Using Same

(71) Applicant: Betty Yu Skin Technology Advisors, LLC, Los Altos, CA (US)

(72) Inventor: Betty Yu, Los Altos, CA (US)

(21) Appl. No.: 18/998,070

(22) PCT Filed: Jul. 24, 2023
(86) PCT No.: PCT/US2023/070805

§ 371 Date: Jan. 23, 2025

Related U.S. Application Data
(60) Provisional application No. 63/392,003, filed on Jul. 25, 2022.

(51) Int. Cl. A61L 26/00; A61K 8/25; A61K 8/891; A61K 9/107; A61K 31/196; A61K 31/351; A61K 31/4174; A61K 31/4745; A61K 31/496; A61K 31/513; A61K 31/573; A61K 31/7036; A61K 33/04; A61K 38/12; A61K 47/02; A61K 47/34

(52) U.S. Cl. CPC A61L 26/0019; A61K 8/25; A61K 8/891; A61K 9/107; A61K 31/196; A61K 31/351; A61K 31/4174; A61K 31/4745; A61K 31/496; A61K 31/513; A61K 31/573; A61K 31/7036; A61K 33/04; A61K 38/12; A61K 47/02; A61K 47/34; A61L 26/0061

(57) ABSTRACT

The present disclosure relates, in part, to cross-linked telechelic polymer compositions and methods of preparation thereof. In another aspect, the present disclosure relates to multilayer compositions comprising an adhesive base (AB) and a mechanically reinforcing composition (MRC), methods of preparation thereof, and methods of use thereof. In certain embodiments, the in situ prepared compositions are suitable for application to the skin of a subject for treating a wound and/or skin condition.

### CROSS-REFERENCE TO RELATED APPLICATIONS

The present application claims priority under 35 U.S.C. § 119(e) to U.S. Provisional Patent Application No. 63/392,003, filed Jul. 25, 2022, which application is incorporated herein by reference in its entirety.

### BACKGROUND OF THE DISCLOSURE

Silicone skin-adhesive compositions have been commercially available for decades, and have been evaluated for use in a multitude of dermatological products that include wound dressings, bandages, medical tapes, transdermal delivery devices, and cosmetics. These products primarily constitute prefabricated cross-linked polysiloxanes that are affixed to the skin upon being removed from a release liner.

More recently, in situ cross-linked, skin-adherent films have been described, wherein polysiloxane reactants and one or more catalysts are stored separately and allowed to react when they are placed in contact on the skin. While these films purport to provide demonstrable skin benefits, such as skin cosmesis and reduction in the skin trans-epidermal water loss, their commercial presence has been limited by a number of factors, including user application error, poor organoleptic qualities, inconveniently lengthy film cure times, poor durability, and short shelf-life and/or storage stability.

There is thus a need in the art for skin-adhesive polysiloxane compositions, methods of preparing the same, and methods of use thereof. The present disclosure addresses this need.

### BRIEF SUMMARY OF THE INVENTION

In one aspect, the present disclosure provides a telechelic polymer composition comprising a cross-linked reaction product of any of.
(i) at least one polysiloxane (a) comprising a number of diorganosiloxy monomers, at least one 1-alkenyl-1-organosiloxy monomer, and two termini, wherein each terminus is independently selected from a triorganosiloxy group and a 1,1-organo-1-alkenyl-siloxy group, and wherein at least three Si atoms present in the polysiloxane are alkenyl-substituted;
(ii) at least one polysiloxane (b) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H);
(iii) at least one polysiloxane (c) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted;
(iv) at least one inert formulation-compatible polysiloxane (d); and
(v) at least one Group X transition metal catalyst.

In certain embodiments, the telechelic polymer composition further comprises:
(vi) at least one additional polysiloxane (f), wherein the at least one additional polysiloxane comprises a number of diorganosiloxy monomers, optionally a 1,1-diorgano-hydrosiloxy monomer, and two termini, wherein each terminus is independently selected from a triorganosiloxy group and a 1,1-diorgano-hydrosiloxy group, and wherein one Si atom present in the polysiloxane is substituted with H (i.e., Si—H).

In certain embodiments, the telechelic polymer composition comprise an adhesive base (AB) composition.

In another aspect, the present disclosure provides a method of preparing the telechelic composition of the present disclosure, the method comprising:
(i) contacting each of the following to provide a first mixture:
   at least one polysiloxane in (a) comprising a number of diorganosiloxy monomers, at least one 1-alkenyl-1-organosiloxy monomer, and two termini, wherein each terminus is independently selected from a triorganosiloxy group and a 1,1-organo-1-alkenyl-siloxy group, and wherein at least three Si atoms present in the polysiloxane are alkenyl-substituted;
   at least one polysiloxane in (b) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H); and
   at least one inert formulation-compatible polysiloxane in (d);
(ii) contacting the first mixture with a Group X transition metal catalyst to provide an at least partially cross-linked mixture; and
(iii) contacting the at least partially cross-linked mixture with:
   at least one polysiloxane in (c) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted.

In another aspect, the present disclosure provides a mechanically reinforcing composition (MRC) comprising:
(i) at least one polysiloxane (g) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted;
(ii) at least one polysiloxane (h) comprising a number of diorganosiloxy monomers, a number of I-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H), optionally wherein at least three Si atoms present in the polysiloxane are substituted with H (i.e., Si—H);
(iii) at least one reinforcing material; and
(iv) at least one silicone miscible, volatile fluid.

In another aspect, the present disclosure provides a multilayer composition comprising:
(a) an adhesive basal layer comprising the adhesive base composition of the present disclosure, wherein the adhesive basal layer optionally further comprises one or more additional compounds and/or compositions selected from the group consisting of a non-reactive fluid, emulsifier, a cosmetic, and a pharmaceutically active agent and/or composition;
(b) a mechanically reinforcing layer comprising the mechanically reinforcing composition of the present disclosure;
   wherein the adhesive basal layer is in contiguous contact with at least a portion of a surface of an object; and
   wherein the mechanically reinforcing layer is in contiguous contact with at least a portion of the surface of the adhesive basal layer.

In another aspect, the present disclosure provides a method for applying a multilayered wound dressing composition to a wound of a subject, the method comprising:
(a) applying to the surface of the wound an adhesive basal layer comprising the adhesive base composition of the present disclosure, wherein the adhesive basal layer optionally further comprises one or more additional compounds and/or compositions selected from the group consisting of a non-reactive fluid, emulsifier, and a pharmaceutically active agent and/or composition; and
(b) applying to the surface of the adhesive basal layer a mechanically reinforcing layer comprising the mechanically reinforced composition of the present disclosure;
   wherein the adhesive basal layer is in contiguous contact with at least a portion of the surface of the wound; and
   wherein the mechanically reinforcing layer is in contiguous contact with at least a portion of the surface of the adhesive basal layer.

In another aspect, the present disclosure provides a method of treating a skin condition and/or wound of a subject, the method comprising:
(a) applying to the surface of the wound an adhesive basal layer comprising the adhesive base composition of the present disclosure, wherein the adhesive basal layer optionally further comprises one or more additional compounds and/or compositions selected from the group consisting of a non-reactive fluid, emulsifier, a cosmetic, and a pharmaceutically active agent and/or composition; and
(b) applying to the surface of the adhesive basal layer a mechanically reinforcing layer comprising the mechanically reinforcing composition of the present disclosure;
   wherein the adhesive basal layer is in contiguous contact with at least a portion of the surface of the wound; and
   wherein the mechanically reinforcing layer is in contiguous contact with at least a portion of the surface of the adhesive basal layer.

In another aspect, the present disclosure provides a kit comprising:
(a) a container comprising the adhesive base composition of the present disclosure, wherein the container is suitable for dispensation;
(b) a container comprising the mechanically reinforcing composition of the present disclosure, wherein the container is suitable for dispensation; and
(c) instructional materials for use thereof.

### BRIEF DESCRIPTION OF THE FIGURES

The drawings illustrate generally, by way of example, but not by way of limitation, various embodiments of the present application.

FIG. 1 provides a graph showing the instantaneous viscosity profile of composition F3-99-1 as a function of time.

FIG. 2 provides a graph showing the instantaneous viscosity profile of composition F3-99-2 after addition of component 2.

FIG. 3 provides a process schematic showing crosslinked telechelic synthesis wherein an inert siloxane brush architecture is incorporated.

FIG. 4 provides a graph showing instantaneous viscosity measurements of Step 2 products of F3-134-1 (i.e., F3-131-1) and F3-133-1 (i.e., F3-132-1).

FIGS. 5A-5C provides photographs showing an improvement in the right and left skin sites before application (FIG. 5A), 48 h (FIG. 5B), and 64 h (FIG. 5C) after initial application of adhesive base (AB) F2-48-2 and mechanical reinforcing composition (MRC) F2-37-1 to eczematous skin at the knee flexural sites of a 2 year old subject.

FIG. 6 provides photographs showing improvement in visible appearance of a facial abrasion over a period of 46 h after application of a crosslinked adherent skin dressing (ASD) comprising F3-102-1 (AB) and F3-96-1 (MRC).

FIGS. 7A-7E provide photographs showing the right leg of an individual self-diagnosed with dry skin with the formulations indicated in Table 20 applied to three distinct sites of the leg, wherein photographs were taken at baseline (i.e., before application; FIG. 7A), 2 min (FIG. 7B), 24 h (FIG. 7C), 48 h (FIG. 7D), and 96 h (FIG. 7E) post application. Sites of application are indicated in FIG. 7B.

FIGS. 8A-8E provide photographs showing the left leg of an individual self-diagnosed with dry skin with the formulations indicated in Table 20 applied to three distinct sites of the leg, wherein photographs were taken at baseline (i.e., before application; FIG. 8A), 2 min (FIG. 8B), 24 h (FIG. 8C), 48 h (FIG. 8D), and 96 h (FIG. 8E) post application. Sites of application are indicated in FIG. 8B.

FIGS. 9A-9C provide photographs showing the right leg of an 85 year old individual experiencing radiation dermatitis after application of F3-136-1 (AB) and F3-115-1 (MRC), wherein photographs were taken at baseline (i.e., before application; FIG. 9A), 2 min (FIG. 9B), and 48 h (FIG. 9C) post application.

FIGS. 10A-10H provide photographs wherein F3-136-1 and F3-115-1 were applied to eczematous lesions present on the dorsal hand region of a 3 year old using a metal roller to apply the AB and a metal spatula to apply the MRC, wherein photographs were taken at baseline (i.e., before application; FIG. 10A), 24 h (FIG. 10B), 48 h (FIG. 10C), and 72 h (FIG. 10D) post application for the left hand, and baseline (FIG. 10E), 24 h (FIG. 10F), 48 h (FIG. 10G), and 72 h (FIG. 10H) post application for the right hand.

FIGS. 11A-11B provide photographs showing the left volar forearm of an individual at baseline (FIG. 11A) and 6 hours after sodium dodecyl sulfate (SDS) patch removal (FIG. 11B). The indicated sites of the forearm were contacted with a SDS patch before or after sequential application of an adhesive base (AB) composition (F5-64-1) and mechanically reinforcing composition (MRC) (F5-66-1).

FIGS. 12A-12B provide photographs showing the right volar forearm of an individual at baseline (FIG. 12A) and 6 hours after sodium dodecyl sulfate (SDS) patch removal (FIG. 12B). The indicated sites of the forearm were contacted with a SDS patch before or after sequential application of an adhesive base (AB) composition (F5-64-1) and mechanically reinforcing composition (MRC) (F5-66-1).

FIG. 13 provides a bar graph depicting average transepidermal water loss (TEWL) value measured at the antecubital fossa (ACF) and outer calf (OC) regions for eight and seven volunteers, respectively, at baseline and one hour after product application for each formulation (i.e., A and B). Product A (AB2-004 and MRC2-003) and Product B (AB4-002 and MRC2-003).

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### DESCRIPTION

In one aspect, the cross-linked telechelic polymers disclosed herein are synthesized utilizing platinum-catalyzed hydrosilylation, first described by Chalk and Harrod in the 1960's. In certain embodiments, cross-linked telechelic polymer synthesis comprises (1) fractional grafting of silicone chains. In certain embodiments, cross-linked telechelic polymer synthesis comprises (2) crosslinking initiation. In certain embodiments, cross-linked telechelic polymer synthesis comprises (3) vinyl capping or reaction termination.

These cross-linked telechelic polymers may be incorporated into multi-layered compositions that are designed to generate micron-thin, in situ cured, adhesive dressings on biological surfaces such as the integumentary system. In certain embodiments, the multi-layered compositions minimally comprise an adhesive base (AB) and a mechanical reinforcing composition (MRC), which upon contact react to form an optically inconspicuous, durable, protective adhesive dressing on the applied surface.

In certain embodiments, the AB comprises at least one cross-linked telechelic polymer and may include a variety of excipients to optimize the adhesion and/or modulate the organoleptic characteristics during application of the formulation. In certain embodiments, the MRC comprises at least one vinyl-functional polysiloxane, at least one hydrido-functional polysiloxane (i.e., Si—H), and at least one reinforcing particle. For both the AB and the MRC, factors such as the reactant molecular weights, the reactive group concentration, the crosslink density, and the excipient chemistries determine the cured adhesive dressing physico-mechanical properties when the AB is placed in contact with the MRC.

For most two-component industrial adhesives, the target pot-life, or working time during which the viscosity of the two components remains low enough to be uniformly mixed and then applied properly to a surface before it can no longer be manipulated, depends on the end user application. For health and beauty applications, a rapid cure time is desired, which then requires a short product pot-life. One consequence of a short pot-life is the increased likelihood of suboptimal mixing of the two components and non-uniform application on the target surface prior to curing, both of which result in variable product performance, including product failure to achieve the touted performance specifications. On the other hand, a long pot-life product calls for stronger user compliance during the curing period such that the adhesives are formed properly.

The relevant formulation composition variables, such as the polymer molecular weight, the vinyl to hydride ratio, the crosslink density and the catalyst concentration, each influence different characteristics of the product performance profile such as spreadability on skin, adhesiveness, elongation, toughness, modulus, and cure rate. Constraints are placed on the range of achievable physico-mechanical properties as a result of the prioritized performance attributes selected in the material optimization scheme.

For single layer compositions, the interdependencies that exist amongst the parameters that define the product performance profile limit the degree to which each parameter may be independently optimized without compromising the performance of the other attributes. For example, greater adhesivity may limit the material toughness.

In addition to those formulation constraints, soft materials that adhere comfortably to the skin while providing the mechanical toughness and durability to endure one's daily routines generally require high molecular weight, and therefore high viscosity, telechelic polymers as precursors. These high viscosity compositions often result in poor organoleptic attributes, including poor on-skin spreadability.

Finally, product stability is essential to ensuring a commercially feasible supply chain to support consumer use. For the platinum-catalyzed hydrosilylation reaction, product stability relies on the sustained Pt activity, which may be subjected to a host of poisoning agents present in the respective composition, or may decline as a result of the formation of colloidal platinum with time.

The application of the novel cross-linked telechelic polymers in these multilayered, in situ cure adhesive dressings enable the independent optimization of adhesivity and mechanical robustness with the development of the AB and the MRC layers, respectively. Thus, a separate mechanical reinforcing layer composition, when combined with the adhesive base layer, dictates the bulk mechanical properties of the dressing such as the modulus, elongation, and/or toughness. Moreover, the multilayer adhesive dressing compositions described by the disclosure provide desirable commercial product attributes such as consistency of user application, favorable organoleptic characteristics, rapid in situ curing at low platinum concentrations and prolonged shelf-life stability.

### Definitions

The term “about” as used herein can allow for a degree of variability in a value or range, for example, within 10%, within 5%, or within 1% of a stated value or of a stated limit of a range, and includes the exact stated value or range.

The term “active agent” as used herein refers to a drug or any compound that is a therapeutic agent or a candidate for use as a therapeutic or as lead compound for designing a therapeutic or that is a known pharmaceutical. Such compounds can be small molecules, including small organic molecules (e.g., anti-microbial agent), peptides, peptide mimetics, antisense molecules, antibodies, fragments of antibodies, recombinant antibodies.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

As used herein, “anti-microbial agent” refers to a molecule or compound suitable for use in a formulation, such as a cosmetic, personal care, paper or textile application, that reduces or prevents microorganism growth. See, for example, U.S. Pat. Nos. 3,202,514 and 3,915,889. Examples of anti-microbial agents include, but are not limited to, sorbic acid and its salts, such as calcium sorbate, sodium sorbate and potassium sorbate, and benzoic acid and its salts, such as calcium benzoate, sodium benzoate and potassium benzoate, natamycin (pimaricin), nisin, and propionic acid and its salts.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “contacting” as used herein refers to bringing two or more materials into close enough proximity such that the two materials can physically interact.

The term “cross-linked” herein refers to a composition containing intermolecular crosslinks and optionally intramolecular crosslinks as well, arising from the formation of covalent bonds. Covalent bonding between two cross-linkable components may be direct (e.g., hydrosilylation of an alkene moiety with a silicon hydride); in which case an atom in one component is directly bound to an atom in the other component, or it may be indirect, through a linking group. A cross-linked matrix may, in addition to covalent bonds, also include intermolecular and/or intramolecular noncovalent bonds such as hydrogen bonds and electrostatic (ionic) bonds. The term “cross-linkable” refers to a component or compound that is capable of undergoing reaction to form a crosslinked composition.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups.

The term “emulsifier” as used herein means a molecule that concentrates at the interface between the phases of an emulsion and reduces the interfacial tension between the phases, thus stabilizing the emulsion.

The term “emulsion” as used herein to a stable suspension of two incompatible fluid materials, where one fluid (such as a liquid) is suspended or dispersed as minute particles or globules in another fluid (for example, oil dispersed in water or silicone dispersed in a carrier fluid).

The term “fluid” as used herein refers to a substance that undergoes continuous deformation when subjected to shear stress.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-vl), and the like.

The term “hydride weight percent” as used herein refers to the percentage mass of the siloxane monomers and/or siloxane termini of a polysiloxane comprising a Si—H moiety as compared to the total mass of the polysiloxane.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “organo” as used herein refers to an organic substituent which is selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted C₆-C₁₀ aryl, optionally substituted phenyl, optionally substituted benzyl, and optionally substituted C₂-C₁₂ heteroaryl. The term “organo” may be used interchangeably with “R”, wherein “R” is defined analogously. The following terms may be used interchangeably with the parenthetical structural definition: diorganosiloxy (—(SiR₂)—O—); 1-alkenyl-1-organo-siloxy ((Si(alkenyl)(R))—O—); 1-organo-hydrosiloxy (—(SiHR)—O—); 1,1-diorgano-1-alkenyl-siloxy (—SiR₂(alkenyl)); triorganosiloxy (—SiR₃); and 1,1-diorgano-hydrosiloxy (—SiR₂H). In certain embodiments, R is a C₁-C₆ alkyl. In certain embodiments, the C₁-C₆ alkyl is linear. In certain embodiments, the C₁-C₆ alkyl is branched. In certain embodiments, R is optionally substituted C₁-C₆ haloalkyl. In certain embodiments, the C₁-C₆ haloalkyl is a C₁-C₆ perfluoroalkyl. In certain embodiments, R is optionally substituted phenyl.

The term “perfluoroalkyl” as used herein refers to an alkyl group in which all of the hydrogen atoms have been replaced by fluorine atoms. Exemplary perfluoroalkyl groups include, but are not limited to, C1-5 perfluoroalkyl, such as trifluoromethyl, pentafluoroethyl, and heptafluoropropyl, inter alia.

The term “polysiloxane” as used herein refers to a polymeric material that comprises a number of linearly arranged siloxane units (i.e., —R₂SiO—) and two termini (i.e., R₃Si—), wherein “R” is selected from H, alkyl, and/or vinyl substituents.

The term “regioregular copolymer” is used to refer to the connectivity of monomer units along the multimeric backbone (e.g., monomer unit A and monomer unit B), where the copolymer comprises two monomer units linked in a regular interleaved manner (i.e., . . . ABABAB . . . ) along the multimeric backbone. In one preferred type of regioregular copolymer, each of the two monomer units is also symmetric along the central axis of the “connectivity” monomer unit between the monomer units.

The term “rheology” as used herein refers to a study of the change in form and flow of matter under the influence of stresses, embracing elasticity, viscosity, and plasticity. For example, when liquids are subjected to stress they will deform irreversibly and flow. The measurement of this flow is the measurement of viscosity.

The term “silicone” and “siloxane” are synonymous. As used herein, the term “siloxane” refers to a class of compounds that include alternate silicon and oxygen atoms, and can include carbon and hydrogen atom substituents. A siloxane contains a repeating silicon-oxygen backbone and can include organic groups attached to a significant proportion of the silicon atoms by silicon-carbon bonds. In commercial silicones most groups are methyl; longer alkyl, fluoroalkyl, phenyl, vinyl, and a few other groups are substituted for specific purposes. Some of the groups also can be hydrogen, chlorine, alkoxy, acyloxy, or alkylamino. These polymers can be combined with fillers, additives, and solvents to result in products classed as silicones. See Kirk-Othmer Encyclopedia of Polymer Science and Technology, Volume 15, John Wiley & Sons, Inc. (New York: 1989), pages 204-209, 234-265, incorporated herein by reference. The siloxanes include any organosilicone polymers or oligomers having a linear or cyclic, branched or crosslinked structure, of variable molecular weight, and essentially based on recurring structural units in which the silicone atoms are linked to each other by oxygen atoms (—Si—O—Si—), and where optionally substituted, substituents can be linked via a carbon atom to the silicone atoms.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “telechelic” is used in the conventional sense to refer to a large molecule (e.g., a polymer or copolymer) that is capped by at least one reactive end group. The preferred telechelic compounds herein are regioregular copolymers having two terminal functional groups, each capable of undergoing further reactions.

The term “uniform” as used herein in the context of one or more coating compositions, is defined herein to mean that the coating composition has a substantially consistent thickness, such that the thickness of the composition measured at position of the film differs from the average thickness of the composition by no more than 25%. In certain embodiments, the composition thickness at a measured position differs from the average thickness of the composition by no more than 10%.

The term “vinyl equivalent” as used herein, refers to the mass (or weight) percentage of siloxane monomers and/or siloxane termini of a polysiloxane as compared to the mass of the polysiloxane.

The term “viscosity” refers to the tendency of a fluid to resist flow and is defined as shear stress divided by shear strain. A fundamental unit of viscosity measurement is the “poise.” A material requiring a shear stress of one dyne per square centimeter to produce a shear rate of one reciprocal second has a viscosity of one poise, or 100 centipoise (cP). Viscosity measurements can be expressed in “Pascal-seconds” (Pa s) or “milli-Pascal-seconds”(mPa s), which are units of the International System and are sometimes used in preference to the Metric designations. One Pascal-second is equal to ten poise; one milli-Pascal-second is equal to one centipoise (P). Conditions used to measure the viscosity should be provided since non-ideal liquids have different values of viscosity for different test conditions of shear rate, shear stress and temperature. The term “kinematic viscosity” as used herein will be referred to simply as viscosity, unless otherwise noted. Kinematic viscosity measurements can be expressed in “centistokes” or “cSt”. The absolute viscosity may be determined by multiplication of the kinematic viscosity by the density of the substance for which the calculation is performed.

### Compositions

### Telechelic Polymer Composition (Adhesive Base)

In one aspect, the present disclosure provides a telechelic polymer composition comprising a cross-linked reaction product of any of:
(a) at least one polysiloxane (a) comprising a number of diorganosiloxy monomers, at least one 1-alkenyl-1-organosiloxy monomer, and two termini, wherein each terminus is independently selected from a triorganosiloxy group and a 1,1-organo-1-alkenyl-siloxy group, and wherein at least three Si atoms present in the polysiloxane are alkenyl-substituted;
(b) at least one polysiloxane (b) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H);
(c) at least one polysiloxane (c) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted;
(d) at least one inert formulation-compatible polysiloxane (d); and
(e) at least one Group X transition metal catalyst.

In certain embodiments, the composition further comprises:
(f) at least one additional polysiloxane (f), wherein the at least one additional polysiloxane comprises a number of diorganosiloxy monomers, optionally a 1,1-diorgano-hydrosiloxy monomer, and two termini, wherein each terminus is independently selected from a triorganosiloxy group and a 1,1-diorgano-hydrosiloxy group, and wherein one Si atom present in the polysiloxane is substituted with H (i.e., Si—H).

In certain embodiments, the at least one polysiloxane (a) possesses at a viscosity ranging from about 800 cSt to about 1200 cSt. In certain embodiments, the at least one polysiloxane (a) possesses an average molecular weight ranging from about 15 kDa to about 45 kDa. In certain embodiments, the at least one polysiloxane (a) possesses an average molecular weight of about 28 kDa. In certain embodiments, the at least one polysiloxane (a) possesses an alkenyl equivalent per kilogram (mol/kg or mmol/g) selected from the group consisting of 0.01, 0.05, 0.10, 0.15, 0.20, 0.25, 0.30, 0.35, 0.40, 0.45, 0.50, 0.55, 0.60, 0.65, and 0.70. In certain embodiments, the at least one polysiloxane (a) possesses a vinyl equivalent per kilogram ranging from about 0.11 to about 0.15. In certain embodiments, the at least one polysiloxane (a) possesses a vinyl equivalent per kilogram ranging from about 0.5 to about 0.7.

In certain embodiments, the at least one polysiloxane (b) possesses a viscosity of about 10,000 cSt. In certain embodiments, the at least one polysiloxane (b) possesses an average molecular weight ranging from about 45 kDa to about 75 kDa. In certain embodiments, the at least one poly siloxane (b) possesses an average molecular weight of about 62.7 kDa. In certain embodiments, the at least one polysiloxane (b) possesses a hydride weight percent selected from the group consisting of 0.001%, 0.002%, 0.003%, 0.004%, 0.005%, 0.006%, 0.007%, 0.008%, 0.009%, and 0.010%. In certain embodiments, the at least one polysiloxane (b) possesses a hydride weight percent of about 0.003%.

In certain embodiments, the at least one polysiloxane (b) possesses at a viscosity ranging from about 7 cSt to about 10 cSt. In certain embodiments, the at least one polysiloxane (a) possesses an average molecular weight ranging from about 1 kDa to about 1.1 kDa. In certain embodiments, the at least one polysiloxane (a) possesses a hydride weight percentage of about 0.18%, 0.19%, or about 0.20%.

In certain embodiments, the at least one polysiloxane (c) possesses a viscosity ranging from about 5,000 cSt to about 170,000 cSt. In certain embodiments, the at least one polysiloxane (c) possesses a vinyl equivalent per kilogram selected from the group consisting of about 0.010, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.030, 0.031, 0.032, 0.033, 0.034, 0.035, 0.036, 0.037, 0.038, 0.039, 0.040.

In certain embodiments, the at least one polysiloxane (f) possesses a viscosity ranging from about 150 cSt to about 250 cSt. In certain embodiments, the at least one polysiloxane (f) possesses an average molecular weight ranging from about 5 kDa to about 15 kDa. In certain embodiments, the at least one polysiloxane (f) possesses a weight fraction ranging from about 0.01% to about 75% of the reactant composition. In certain embodiments, the at least one polysiloxane (f) possesses a weight fraction selected from the group consisting of about 0.01%, 0.05%, 0.10%, 0.50%, 1.0%, 2.0%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0%, 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, and 75.0%.

In certain embodiments, the at least one polysiloxane (a) is a compound of formula (Ia):

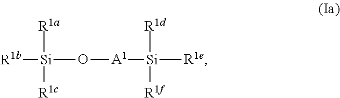

wherein:
A¹ comprises m units of

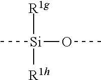

monomer and n units of

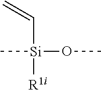

monomer, wherein each ---- bond is a Si—O bond;
m is an integer ranging from 410 to 470;
n is an integer ranging from 1 to 50;
R^{1a}, R^{1b}, R^{1c}, R^{1d}, R^{1e}, and R^{1f} are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, optionally substituted C₂-C₁₂ heteroaryl, and optionally substituted C₂-C₆ alkenyl,
   wherein R^{1a}, R^{1b}, R^{1c}, R^{1d}, R^{1e}, and R^{1f} are selected such that each Si atom is substituted with no more than one optionally substituted C₂-C₆ alkenyl; and
R^{1g}, R^{1h}, and R¹ⁱ are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl.

In certain embodiments, R^{1a}, R^{1b}, R^{1c}, R^{1d}, R^{1e}, R^{1f}, R^{1g}, R^{1h}, R¹ⁱ, m, and n are selected such that the at least one polysiloxane (a) possesses a viscosity ranging from about 800 cSt to about 1200 cSt. In certain embodiments, R^{1a}, R^{1b}, R^{1c}, R^{1d}, R^{1e}, R^{1f}, R^{1g}, R^{1h}, R¹ⁱ, m, and n are selected such that the at least one polysiloxane (a) possesses an average molecular weight ranging from about 15 kDa to about 45 kDa. In certain embodiments, R^{1a}, R^{1b}, R^{1c}, R^{1d}, R^{1e}, R^{1f}, R^{1g}, R^{1h}, R¹ⁱ, m, and n are selected such that the at least one polysiloxane (a) possesses an average molecular weight of about 28 kDa. In certain embodiments, R^{1a}, R^{1b}, R^{1c}, R^{1d}, R^{1e}, R^{1f}, R^{1g}, R^{1h}, R¹ⁱ, m, and n are selected such that the at least one polysiloxane (a) possesses an alkenyl equivalent per kilogram selected from the group consisting of 0.01, 0.05, 0.10, 0.15, 0.20, 0.25, 0.30, 0.35, 0.40, 0.45, 0.50, 0.55, 0.60, 0.65, and 0.70.

In certain embodiments, the at least one polysiloxane (b) is a compound of formula (Ib):

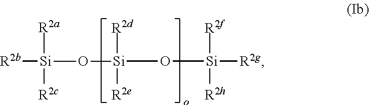

wherein:
R^{2a}, R^{2b}, R^{2c}, R^{2f}, R^{2g} and R^{2h} are each independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
each occurrence of R^{2d} and R^{2e} is independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
o is an integer ranging from 500 to 1500; and
wherein R^{2a}, R^{2b}, R^{2c}, R^{2d}, R^{2e}, R^{2f}, R^{2g} and R^{2h} are selected such that each Si atom is substituted with no more than one H atom.

In certain embodiments, R^{2a}, R^{2b}, R^{2c}, R^{2d}, R^{2e}, R^{2f}, R^{2g}, R^{2h}, and o are selected such that the at least one polysiloxane (b) possesses a viscosity of about 10,000 cSt. In certain embodiments, R^{2a}, R^{2b}, R^{2c}, R^{2d}, R^{2e}, R^{2f}, R^{2g}, R^{2h}, and o are selected such that the at least one polysiloxane (b) possesses an average molecular weight ranging from about 45 kDa to about 75 kDa. In certain embodiments, R^{2a}, R^{2b}, R^{2c}, R^{2d}, R^{2e}, R^{2f}, R^{2g}, R^{2h}, and o are selected such that the at least one polysiloxane (b) possesses an average molecular weight of about 62.7 kDa. In certain embodiments, R^{2a}, R^{2b}, R^{2c}, R^{2d}, R^{2e}, R^{2f}, R^{2g}, R^{2h}, and o are selected such that the at least one polysiloxane (b) possesses a hydride weight percent selected from the group consisting of about 0.001%, 0.002%, 0.003%, 0.004%, 0.005%, 0.006%, 0.007%, 0.008%, 0.009%, and about 0.01%.

In certain embodiments, R^{2a}, R^{2b}, R^{2c}, R^{2d}, R^{2e}, R^{2f}, R^{2g}, R^{2h}, and o are selected such that the at least one polysiloxane (b) possesses a viscosity of about 7 cSt, 8 cSt, 9 cSt, or about 10 cSt. In certain embodiments, R^{2a}, R^{2b}, R^{2c}, R^{2d}, R^{2e}, R^{2f}, R^{2g}, R^{2h}, and o are selected such that the at least one polysiloxane (b) possesses an average molecular weight ranging from about 1.0 kDa to about 1.1 kDa. In certain embodiments, R^{2a}, R^{2b}, R^{2c}, R^{2d}, R^{2e}, R^{2f}, R^{2g}, R^{2h}, and o are selected such that the at least one polysiloxane (b) possesses an average molecular weight of about 62.7 kDa. In certain embodiments, R^{2a}, R^{2b}, R^{2c}, R^{2d}, R^{2e}, R^{2f}, R^{2g}, R^{2h}, and o are selected such that the at least one polysiloxane (b) possesses a hydride weight percent selected from the group consisting of about 0.18, 0.19, or about 0.20%.

In certain embodiments, the at least one polysiloxane (c) is a compound of formula (Ic):

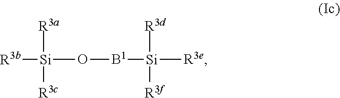

wherein:
B¹ comprises p units of

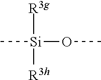

monomer and q units of

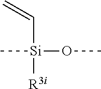

monomer,
   wherein each ---- bond is a Si—O bond;
R^{3a}, R^{3b}, R^{3c}, R^{3d}, R^{3e}, and R^{3f} are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, optionally substituted C₂-C₁₂ heteroaryl, and optionally substituted C₂-C₆ alkenyl,
   wherein R^{3a}, R^{3b}, R^{3c}, R^{3d}, R^{3e}, and R^{3f} are selected such that each Si atom is substituted with no more than one optionally substituted C₂-C₆ alkenyl;
R^{3g}, R^{3h}, and R³ⁱ are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
p is an integer ranging from 500 to 2000; and
q is an integer ranging from 0 to 50.

In certain embodiments, R^{3a}, R^{3b}, R^{3c}, R^{3d}, R^{3e}, R^{3f}, p, and q, are selected such that the at least one polysiloxane (c) possesses a viscosity ranging from about 5,000 cSt to about 170,000 cSt. In certain embodiments, R^{3a}, R^{3b}, R^{3c}, R^{3d}, R^{3e}, R^{3f}, p, and q, are selected such that the at least one polysiloxane (c) possesses an alkenyl equivalent per kilogram selected from the group consisting of about 0.010, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.030, 0.031, 0.032, 0.033, 0.034, 0.035, 0.036, 0.037, 0.038, 0.039, 0.040.

In certain embodiments, the at least one polysiloxane (f) is a compound of formula (If):

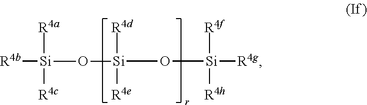

wherein:
R^{4a}, R^{4b}, R^{4c}, R^{4f}, and R^{4g} are each independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
each occurrence of R^{4d} and R^{4e} is independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
r is an integer ranging from 100 to 400; and
wherein no more than one of R^{4a}, R^{4b}, R^{4c}, R^{4d}, R^{4e}, R^{4f}, R^{4g}, and R^{4h} is H.

In certain embodiments, R^{4a}, R^{4b}, R^{4c}, R^{4d}, R^{4e}, R^{4f}, R^{4g}, R^{4h}, and r are selected such that the at least one polysiloxane (f) possesses a viscosity ranging from about 150 cSt to about 250 cSt. In certain embodiments, R^{4a}, R^{4b}, R^{4c}, R^{4d}, R^{4e}, R^{4f}, R^{4g}, R^{4h}, and r are selected such that the at least one polysiloxane (f) possesses an average molecular weight ranging from about 5 kDa to about 15 kDa. In certain embodiments, R^{4a}, R^{4b}, R^{4c}, R^{4d}, R^{4e}, R^{4f}, R^{4g}, R^{4h}, and r are selected such that the at least one polysiloxane (f) possesses an average molecular weight of about 10 kDa. In certain embodiments, R^{4a}, R^{4b}, R^{4c}, R^{4d}, R^{4e}, R^{4f}, R^{4g}, R^{4h}, and r are selected such that the at least one polysiloxane (f) possesses a weight fraction ranging from about 0.01% to about 75% of the reactant composition.

In certain embodiments, the Group X catalyst comprises Pt. In certain embodiments, the Pt is Pt(0). In certain embodiments, the Group X catalyst is Karstedt's catalyst:

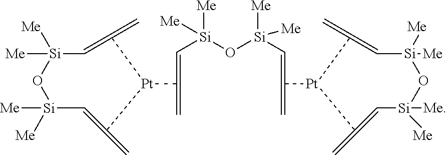

In certain embodiments, the inert formulation-compatible polysiloxane is a compound of formula (Id):

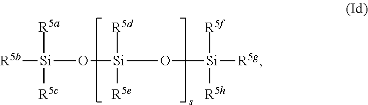

wherein:
R^{5a}, R^{5b}, R^{5c}, R^{5d}, R^{5e}, R^{5f}, R^{5g}, and R^{5h} are each independently selected from the group consisting of C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl; and
s is an integer ranging from 1 to about 500.

In certain embodiments, the inert formulation-compatible polysiloxane is selected from the group consisting of polydimethylsiloxane, dimethiconol, disiloxane, trisiloxane, and diphenyl dimethicone/vinyl diphenyl dimethicone/silsesquioxane cross-polymer.

In certain embodiments, the inert formulation-compatible polysiloxane is decamethylcyclopentasiloxane.

In certain embodiments, R^{1a}, R^{1b}, R^{1c}, R^{1d}, R^{1e}, R^{1f}, R^{1g}, R^{1h}, and R¹ⁱ are each independently CH₃ or CH═CH₂. In certain embodiments, R^{2a}, R^{2b}, R^{2c}, R^{2d}, R^{2e}, R^{2f}, R^{2g}, and R^{2h} are each independently H or CH₃. In certain embodiments, R^{3a}, R^{3b}, R^{3c}, R^{3d}, R^{3e}, and R^{3f} are each independently CH₃ or CH═CH₂. In certain embodiments, R^{3g}, R^{3h}, and R³ⁱ are each independently CH₃ or CH═CH₂. In certain embodiments, R^{4a}, R^{4b}, R^{4c}, R^{4d}, R^{4e}, R^{4f}, R^{4g}, and R^{4h} are each independently H or CH₃. In certain embodiments, R^{5a}, R^{5b}, R^{5c}, R^{5d}, R^{5e}, R^{5f}, R^{5g}, and R^{5h} are each independently CH₃.

In certain embodiments, the composition further comprises at least one additive.

In certain embodiments, additive is at least one selected from the group consisting of glycerin, cetyl diglyceryl tris(trimethylsiloxy)silylethyl dimethicone, hexamethyldisilazane (HMDS) fumed silica, and polyoxyethylene/polyoxypropylene copolymer (PEG/PPG-18/18 dimethicone).

In certain embodiments, the composition has a viscosity selected from the group consisting of about 4000, 5000, 6000, 7000, 8000, 9000, 10000, 15000, 20000, 25000, 30000, 35000, 40000, 45000, 50000, 55000, 60000, 65000, 70000, 75000, 80000, 85000, 90000, 95000 and about 100000 cSt.

In certain embodiments, the composition has a total ratio of units of silicon hydride (i.e., Si—H) to vinyl-substituted silicon (i.e., Si—C(H)═CH₂) in all reactant components selected from the group consisting of 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, and about 0.80.

In certain embodiments, the at least one polysiloxane (a) comprises trimethylsiloxy terminated, 0.8-1.2% vinylmethylsiloxane dimethylsiloxane copolymer. In certain embodiments, the at least one polysiloxane (b) comprises hydride terminated polydimethylsiloxane. In certain embodiments, the at least one polysiloxane (c) comprises vinyl terminated poly dimethylpolysiloxane. In certain embodiments, the at least one inert formulation-compatible polysiloxane comprises polydimethy siloxane and/or decamethylcyclopentasiloxane. In certain embodiments, the at least one Group X transition metal catalyst comprises Karstedt's catalyst. In certain embodiments, the at least one additional polysiloxane (f) comprises monohydride terminated polydimethylpolysiloxane.

In certain embodiments, the at least one polysiloxane (a) comprises about 6.0% to about 12.0% of the composition by weight (w/w %). In certain embodiments, the at least one polysiloxane (b) comprises about 6.0% to about 12.0% of the composition by weight (w/w %). In certain embodiments, the at least one polysiloxane (c) comprises about 20.0% to about 30.0% of the composition by weight (w/w %). In certain embodiments, the at least one formulation-compatible polysiloxane comprises about 10.0% to about 60.0% of the composition by weight (w/w %). In certain embodiments, the at least one Group X catalyst comprises about 0.001% (10 ppm) to about 0.02% (200 ppm) of the composition by weight (w/w %). In certain embodiments, the at least one additional polysiloxane (f) comprises about 1.0% to about 10.0% of the composition by weight (w/w %).

In certain embodiments, the at least one polysiloxane (a) comprises about 9.0% of the composition by weight (w/w %). In certain embodiments, the at least one polysiloxane (b) comprises about 9.4% of the composition by weight (w/w %). In certain embodiments, the at least one polysiloxane (c) comprises about 24.2% of the composition by weight (w/w %). In certain embodiments, the at least one formulation-compatible polysiloxane comprises about 49% of the composition by weight (w/w %). In certain embodiments, the at least one Group X catalyst comprises about 0.0048% (48 ppm) of the composition by weight (w/w %). In certain embodiments, the at least one additional polysiloxane (f) comprises about 5.8% of the composition by weight (w/w %).

In certain embodiments, the at least one polysiloxane (a) comprises about 14.0% to about 20.0% of the composition by weight (w/w %). In certain embodiments, the at least one polysiloxane (b) comprises about 0.20% to about 0.40% of the composition by weight (w/w %). In certain embodiments, the at least one polysiloxane (c) comprises about 18% to about 40% of the composition by weight (w/w %). In certain embodiments, the at least one formulation-compatible polysiloxane comprises about 35% to about 50% of the composition by weight (w/w %). In certain embodiments, the at least one Group X catalyst comprises about 0.0080% (80 ppm) to about 0.0120% (120 pm) of the composition by weight (w/w %). In certain embodiments, the at least one additional polysiloxane (f) comprises about 7% to about 12% of the composition by weight (w/w %).

In certain embodiments, the at least one polysiloxane (a) comprises about 15.5% of the composition by weight (w/w %). In certain embodiments, the at least one polysiloxane (b) comprises about 0.30% of the composition by weight (w/w %). In certain embodiments, the at least one polysiloxane (c) comprises about 35.5% of the composition by weight (w/w %). In certain embodiments, the at least one formulation-compatible polysiloxane comprises about 37.3% of the composition by weight (w/w %). In certain embodiments, the at least one Group X catalyst comprises about 0.0090% (90 ppm) of the composition by weight (w/w %). In certain embodiments, the at least one additional polysiloxane (f) comprises about 8.5% of the composition by weight (w/w %).

In certain embodiments, the at least one polysiloxane (a) comprises about 18.9% of the composition by weight (w/w %). In certain embodiments, the at least one polysiloxane (b) comprises about 0.30% of the composition by weight (w/w %). In certain embodiments, the at least one polysiloxane (c) comprises about 21.60% of the composition by weight (w/w %). In certain embodiments, the at least one formulation-compatible polysiloxane comprises about 45.1% of the composition by weight (w/w %). In certain embodiments, the at least one Group X catalyst comprises about 0.0108% (108 ppm) of the composition by weight (w/w %). In certain embodiments, the at least one additional polysiloxane (f) comprises about 10.4% of the composition by weight (w/w %).

In certain embodiments, the at least one polysiloxane (a) comprises about 17.18% of the composition by weight (w/w %). In certain embodiments, the at least one polysiloxane (b) comprises about 0.26% of the composition by weight (w/w %). In certain embodiments, the at least one polysiloxane (c) comprises about 19.69% of the composition by weight (w/w %). In certain embodiments, the at least one formulation-compatible polysiloxane comprises about 44.00% of the composition by weight (w/w %). In certain embodiments, the at least one Group X catalyst comprises about 0.0099% (99 ppm) of the composition by weight (w/w %). In certain embodiments, the at least one additional polysiloxane (f) comprises about 9.45% of the composition by weight (w/w %).

In certain embodiments, the telechelic polymer composition (adhesive base) comprises AB6-001 (Table 24). In certain embodiments, the telechelic polymer composition (adhesive base) comprises AB6-001 (Table 24), wherein each component has a weight percent variance of about ±0.10%.

In certain embodiments, the telechelic polymer composition (adhesive base) consists essentially of AB6-001 (Table 24). In certain embodiments, the telechelic polymer composition (adhesive base) consists essentially of AB6-001 (Table 24), wherein each component has a weight percent variance of about ±0.1%.

In certain embodiments, the telechelic polymer composition (adhesive base) comprises F5-41-1A (Tables 25a-25b). In certain embodiments, the telechelic polymer composition (adhesive base) comprises F5-41-1A (Tables 25a-25b), wherein each component has a weight percent variance of about ±0.1%.

In certain embodiments, the telechelic polymer composition (adhesive base) consists essentially of F5-41-1A (Tables 25a-25b). In certain embodiments, the telechelic polymer composition (adhesive base) consists essentially of F5-41-1A (Tables 25a-25b), wherein each component has a weight percent variance of about ±0.1%.

In certain embodiments, the telechelic polymer composition (adhesive base) comprises F5-78-2 (Table 29). In certain embodiments, the telechelic polymer composition (adhesive base) comprises F5-78-2 (Table 29), wherein each component has a weight percent variance of about ±0.1%.

In certain embodiments, the telechelic polymer composition (adhesive base) consists essentially of F5-78-2 (Table 29). In certain embodiments, the telechelic polymer composition (adhesive base) consists essentially of F5-78-2 (Table 29), wherein each component has a weight percent variance of about ±0.1%.

In certain embodiments, in the telechelic polymer composition (adhesive base) of formula AB6-001, F5-41-1A, or F5-78-2 the fumed silica comprises about 4% to about 10% of the composition (w/w %). In certain embodiments, in the telechelic polymer composition (adhesive base) of formula AB6-001, F5-41-1A, or F5-78-2 the composition may further comprise at least one additive. In certain embodiments, the at least one additive comprises about 0.25% to about 2% of the composition (w/w %).

In certain embodiments, each occurrence of optionally substituted alkyl, optionally substituted cycloalkyl, optionally substituted alkenyl, optionally substituted heteroalkyl, optionally substituted heterocycloalkyl, optionally substituted benzyl, optionally substituted aryl, optionally substituted heteroaryl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH. N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

### Mechanically Reinforcing Composition (MRC)

In developing a mechanical reinforcing composition, the target performance attributes included rapid curing (e.g., within 2 minutes), mechanical toughness to provide lasting durability, >150% elongation to complement comfortable skin movement, and a Young's modulus allowing for natural skin aesthetics. Moreover, an ideal viscosity was proposed to be sufficiently low so as to enable dispensation from commercial packaging. Because of the viscosity requirement for formulation dispensing, low viscosity formulation components were screened, using a semiquantitative assessment of the resulting material mechanical properties.

In another aspect, the present disclosure provides a mechanically reinforcing composition (MRC) comprising:
(i) at least one polysiloxane (g) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted;
(ii) at least one polysiloxane (h) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H), optionally wherein at least three Si atoms present in the polysiloxane are substituted with H (i.e., Si—H);
(iii) at least one reinforcing material; and
(iv) at least one silicone miscible, volatile fluid.

In certain embodiments, the composition further comprises at least one non-volatile silicone miscible fluid.

In certain embodiments, the at least one polysiloxane (g) is a compound of formula (Ig):

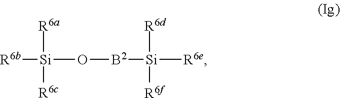

wherein:
B² comprises t units of

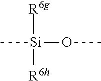

monomer and u units of

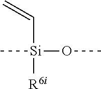

monomer,
   wherein each ---- bond is a Si—O bond;
R^{6a}, R^{6b}, R^{6c}, R^{6d}, R^{6e}, and R^{6f} are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, optionally substituted C₂-C₁₂ heteroaryl, and optionally substituted C₂-C₆ alkenyl,
   wherein R^{6a}, R^{6b}, R^{6c}, R^{6d}, R^{6e}, and R^{6f} are selected such that each Si atom is substituted with no more than one optionally substituted C₂-C₆ alkenyl;
R^{6g}, R^{6h}, and R⁶ⁱ are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
t is an integer ranging from 300 to 2000; and
u is an integer ranging from 0 to 50.

In certain embodiments, the at least one polysiloxane (h) is a compound of formula (Ih):

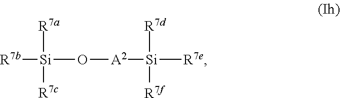

A² comprises v units of

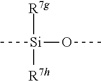

monomer and w units of

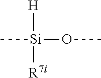

monomer,
   wherein each ---- bond is a Si—O bond;
R^{7a}, R^{7b}, R^{7c}, R^{7d}, R^{7e}, and R^{7f} are each independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl,
   wherein R^{7a}, R^{7b}, R^{7c}, R^{7d}, R^{7e}, and R^{7f} are selected such that each Si atom is substituted with no more than one H atom;
each occurrence of R^{7g}, R^{7h}, and R⁷ⁱ is independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
v is an integer ranging from 10 to 500; and
w is an integer ranging from 2 to 10.

In certain embodiments, R^{6a}, R^{6b}, R^{6c}, R^{6d}, R^{6e}, and R^{6f} are each independently selected from the group consisting of CH₃ and —CH═CH₂. In certain embodiments, R⁶⁹, R^{6h} and R⁶ⁱ are each independently CH₃.

In certain embodiments, R^{7a}, R^{7b}, R^{7c}, R^{7d}, R^{7e}, and R^{7f} are each independently selected from the group H and CH₃ R^{6g}, R^{6h}, and R⁶ⁱ are each independently CH₃. In certain embodiments, R^{7g}, R^{7h}, and R⁷ⁱ are each independently CH₃.

In certain embodiments, the silicone miscible, volatile fluid is at least one selected from the group consisting of disiloxane, trisiloxane, and decamethyl cyclopentasiloxane.

In certain embodiments, the reinforcing agent is at least one selected from the group consisting of silica and HMDS treated fumed silica.

In certain embodiments, wherein the composition has a total ratio of units of silicon hydride (i.e., Si—H) to vinyl-substituted silicon (i.e., Si—C(H)═CH₂) in all reactant components is selected from the group consisting of about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, and about 20:1.

In certain embodiments, the composition further comprises one or more additives. In certain embodiments, the additive is a rheology modifier.

In certain embodiments, the additive is an aesthetic and/or cosmetic modifier. In certain embodiments, the aesthetic and/or cosmetic modifier is at least one selected from the group consisting of vitamin A, vitamin B3, vitamin C, vitamin D, vitamin E, vitamin F, vitamin K, glycolic acid, sunscreen, and/or panthenol.

In certain embodiments, the additive is a pharmaceutically active compound and/or composition.

In certain embodiments, the pharmaceutically active additive is at least one selected from the group consisting of one or more steroids (e.g., mometasone, clobetasol, triamcinolone, fluocinonide, flurandrenolide, clocortolone, halobetasol, desoximetasone, desonide, hydrocortisone, betamethasone, fluticasone, halcinonide, fluocinolone, prednicarbate, diflorasone, flurandrenolide, amcinonide and alclometasone), one or more retinoids (e.g., tretinoin, adapalene, tazarotene, alitretinoin and bexarotene), benzoyl peroxide, azelaic acid, diamino-diphenyl sulphone, one or more JAK inhibitors (e.g., ruxolitinib and delgocitinib), one or more antibiotics (e.g., fusidic acid, mupirocin, retapamulin, silver sulfadiazine, bacitracin, neomycin, polymyxin B, sulfacetamide sodium, sulfur, ozenoxacin, silver sulfadiazine, erythromycin, mafenide, gentamicin, clindamycin, metronidazole, gentamicin, and nadifloxacin), one or more calcineurin inhibitors (e.g., tacrolimus and pimecrolimus), one or more antifungals (e.g., clotrimazole, terbinafine, miconazole, econazole, ketoconazole, tioconazole and amorolfine), becaplermin, 5-fluorouracil, diclofenac, and imiquimod.

In certain embodiments, the at least one polysiloxane (g) comprises vinyl terminated dimethylpolysiloxane. In certain embodiments, the at least one polysiloxane (h) comprises trimethylsiloxy terminated, pendant silicon-hydride functional polydimethylsiloxane. In certain embodiments, the at least one reinforcing material comprises silica and HMDS treated fumed silica. In certain embodiments, the at least one silicone miscible, volatile fluid comprises decamethyl cyclopentasiloxane.

In certain embodiments, the at least one polysiloxane (g) comprises about 30% to about 50% of the composition by weight (w/w %). In certain embodiments, the at least one polysiloxane (h) comprises about 1% to about 10% of the composition by weight (w/w %). In certain embodiments, the at least one reinforcing material comprise about 10% to about 30% of the composition by weight (w/w %). In certain embodiments, the at least one silicone miscible, volatile fluid comprise about 35% to about 50% of the composition by weight.

In certain embodiments, the at least one polysiloxane (g) comprises about 36.6% of the composition by weight (w/w %). In certain embodiments, the at least one polysiloxane (h) comprises about 6.2% of the composition by weight (w/w %). In certain embodiments, the at least one reinforcing material comprise about 14.5% of the composition by weight (w/w %). In certain embodiments, the at least one silicone miscible, volatile fluid comprise about 42.7% of the composition by weight.

In certain embodiments, the at least one polysiloxane (g) comprises vinyl terminated dimethylpolysiloxane. In certain embodiments, the at least one polysiloxane (h) comprises trimethylsiloxy terminated, pendant silicon-hydride functional polydimethylsiloxane. In certain embodiments, the at least one reinforcing material comprises silica and HMDS treated fumed silica. In certain embodiments, the at least one silicone miscible, volatile fluid comprises decamethyl cyclopentasiloxane. In certain embodiments, the at least one non-volatile silicone miscible fluid comprises polydimethylsiloxane fluid.

In certain embodiments, the at least one polysiloxane (g) comprises about 20% to about 40% of the composition by weight (w/w %). In certain embodiments, the at least one polysiloxane (h) comprises about 1% to about 10% of the composition by weight (w/w %). In certain embodiments, the at least one reinforcing material comprises about 10% to about 30% of the composition by weight (w/w %). In certain embodiments, the at least one silicone miscible, volatile fluid comprises about 35% to about 50% of the composition by weight (w/w %). In certain embodiments, the at least one non-volatile silicone miscible fluid comprises about 0.1 to about 5% of the composition by weight (w/w %).

In certain embodiments, the at least one polysiloxane (g) comprises about 30% of the composition by weight (w/w %). In certain embodiments, the at least one polysiloxane (h) comprises about 5% of the composition by weight (w/w %). In certain embodiments, the at least one reinforcing material comprises about 22% of the composition by weight (w/w %). In certain embodiments, the at least one silicone miscible, volatile fluid comprises about 42% of the composition by weight (w/w %). In certain embodiments, the at least one non-volatile silicone miscible fluid comprises about 0.7% of the composition by weight (w/w %).

In certain embodiments, the at least one polysiloxane (g) comprises about 34.4%, 34.8%, or about 36.2% of the composition by weight (w/w %). In certain embodiments, the at least one polysiloxane (h) comprises about 5.8%, 5.9%, or about 6.1% of the composition by weight (w/w %). In certain embodiments, the at least one reinforcing material comprises about 14.4%, 18.6%, or about 18.8% of the composition by weight (w/w %). In certain embodiments, the at least one silicone miscible, volatile fluid comprises about 40.1%, 40.6%, or about 42.3% of the composition by weight (w/w %). In certain embodiments, the at least one non-volatile silicone miscible fluid comprises about 0.0%, 0.1%, 0.2%, 0.3%, 0.4%, 0.5%, 0.6%, 0.7%, 0.8%, 0.9%, or about 1.0% of the composition by weight (w/w %).

In certain embodiments, the mechanically reinforcing composition comprises F5-57-1 (Table 39). In certain embodiments, the mechanically reinforcing composition consists essentially of F5-57-1 (Table 39).

In certain embodiments, the mechanically reinforcing composition comprises F5-62-1 (Tables 42a-42b). In certain embodiments, the mechanically reinforcing composition consists essentially of F5-62-1 (Tables 42a-42b).

In certain embodiments, the mechanically reinforcing composition comprises F5-62-2 (Tables 42a-42b). In certain embodiments, the mechanically reinforcing composition consists essentially of F5-62-2 (Tables 42a-42b).

In certain embodiments, the mechanically reinforcing composition comprises F5-66-1 (Tables 42a-42b). In certain embodiments, the mechanically reinforcing composition consists essentially of F5-66-1 (Tables 42a-42b).

In certain embodiments, the silica (e.g., Goddball G-6C, Iwase Cosfa) comprises about 4% to about 10% of the composition (w/w %). In certain embodiments, the polydimethylsiloxane fluid comprises about 0% to about 5% of the composition (w/w %).

In certain embodiments, the mechanically reinforcing composition of formula F5-57-1, F5-62-1, F5-62-2, or F5-66-1 further comprises at least one additive. In certain embodiments, the at least one polysiloxane (g) comprises two vinyl terminated dimethylpolysiloxanes. In certain embodiments, the at least one polysiloxane (h) comprises two trimethylsiloxy terminated, pendant silicon-hydride functional polydimethylsiloxanes. In certain embodiments, the at least one reinforcing material comprises silica and HMDS treated fumed silica. In certain embodiments, the at least one silicone miscible, volatile fluid comprises decamethyl cyclopentasiloxane.

In certain embodiments, the at least one polysiloxane (g) comprises about 20% to about 40% of the composition by weight (w/w %). In certain embodiments, the at least one polysiloxane (h) comprises about 1% to about 10% of the composition by weight (w/w %). In certain embodiments, the at least one reinforcing material comprises about 5% to about 25% of the composition by weight (w/w %). In certain embodiments, the at least one silicone miscible, volatile fluid comprises about 40% to about 60% of the composition by weight (w/w %).

In certain embodiments, the at least one polysiloxane (g) comprises about 27% of the composition by weight (w/w %). In certain embodiments, the at least one polysiloxane (h) comprises about 6% of the composition by weight (w/w %). In certain embodiments, the at least one reinforcing material comprises about 13% of the composition by weight (w/w %). In certain embodiments, the at least one silicone miscible, volatile fluid comprises about 54% of the composition by weight (w/w %).

### Multilayer Composition

In another aspect, the present disclosure provides a multilayer composition comprising:
(a) an adhesive basal layer comprising the composition of the present disclosure, wherein the adhesive basal layer optionally further comprises one or more additional compounds and/or compositions selected from the group consisting of a non-reactive fluid, emulsifier, a cosmetic, and a pharmaceutically active agent and/or composition;
(b) a mechanically reinforcing layer comprising the composition of the present disclosure;
wherein the adhesive basal layer is in contiguous contact with at least a portion of a surface of an object; and
wherein the mechanically reinforcing layer is in contiguous contact with at least a portion of the surface of the adhesive basal layer.

### Emulsion Composition

In another aspect, the present disclosure provides an emulsion composition comprising:
(a) at least one cross-linked telechelic polymer composition,
   wherein the at least one cross-linked telechelic polymer composition comprises about 1% to about 25% of the emulsion composition (w/w %);
(b) at least one emulsifier,
   wherein the at least one emulsifier comprises about 0.10% to about 10% of the emulsion composition (w/w %);
(c) at least one polar solvent or water-miscible solvent,
   wherein the at least one polar solvent or water-miscible solvent comprises about 50% to about 99% of the emulsion composition (w/w %); and
(d) at least one silicone fluid,
   wherein the at least one silicone fluid comprises about 1% to about 25% of the emulsion composition (w/w %).

In certain embodiments, the at least one cross-linked telechelic polymer composition is an adhesive base composition of the present disclosure.

In certain embodiments, the at least one cross-linked telechelic polymer composition comprises about 10% of the emulsion composition (w/w %).

In certain embodiments, at least one emulsifier comprises cetyl diglyceryl tris(trimethylsiloxy)silylethyl dimethicone.

In certain embodiments, the at least one emulsifier comprises about 1% of the emulsion composition (w/w %).

In certain embodiments, the at least one polar solvent or water-miscible solvent comprises at least one selected from the group consisting of 1,3-butylene glycol and glycerin.

In certain embodiments, the at least one polar solvent or water-miscible solvent comprises about 75% of the emulsion composition (w/w %).

In certain embodiments, the at one silicone fluid comprises at least one selected from the group consisting of caprylyl methicone and dimethicone.

In certain embodiments, the at least one silicone fluid comprises about 14% of the emulsion composition (w/w %).

### Methods

### Telechelic Polymer Composition (Adhesive Base)

In another aspect, the present disclosure provides a method of preparing a composition of the present disclosure, the method comprising:
(i) contacting each of the following to provide a first mixture:
   at least one polysiloxane (a) comprising a number of diorganosiloxy monomers, at least one 1-alkenyl-1-organosiloxy monomer, and two termini, wherein each terminus is independently selected from a triorganosiloxy group and a 1,1-organo-1-alkenyl-siloxy group, and wherein at least three Si atoms present in the polysiloxane are alkenyl-substituted;
   at least one polysiloxane (b) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H); and
   at least one inert formulation-compatible polysiloxane (d);
(ii) contacting the first mixture with a Group X transition metal catalyst to provide an at least partially cross-linked mixture; and
(iii) contacting the at least partially cross-linked mixture with:
   at least one polysiloxane (c) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted.

In another aspect, the present disclosure provides a method of preparing a composition of the present disclosure, the method comprising:
(i) contacting each of the following to provide a first mixture:
   at least one polysiloxane (a) comprising a number of diorganosiloxy monomers, at least one 1-alkenyl-1-organosiloxy monomer, and two termini, wherein each terminus is independently selected from a triorganosiloxy group and a 1,1-organo-1-alkenyl-siloxy group, and wherein at least three Si atoms present in the polysiloxane are alkenyl-substituted; and
   at least one polysiloxane (b) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H);
(ii) contacting the first mixture with at least one inert formulation-compatible polysiloxane (d) and a Group X transition metal catalyst to provide an at least partially cross-linked mixture; and
(iii) contacting the at least partially cross-linked mixture with:
   at least one polysiloxane (c) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted.

In another aspect, the present disclosure provides a method of preparing a composition of the present disclosure, the method comprising:
(i) contacting each of the following to provide a first mixture:
   at least one polysiloxane (a) comprising a number of diorganosiloxy monomers, at least one 1-alkenyl-1-organosiloxy monomer, and two termini, wherein each terminus is independently selected from a triorganosiloxy group and a 1,1-organo-1-alkenyl-siloxy group, and wherein at least three Si atoms present in the polysiloxane are alkenyl-substituted;
   least one polysiloxane (f) comprising a number of diorganosiloxy monomers, optionally a 1,1-diorgano-hydrosiloxy monomer, and two termini, wherein each terminus is independently selected from a triorganosiloxy group and a 1,1-diorgano-hydrosiloxy group, and wherein one Si atom present in the polysiloxane is substituted with H (i.e., Si—H);
   at least one inert formulation-compatible polysiloxane (d); and
   at least one Group X transition metal catalyst;
(ii) contacting the first mixture with the following to provide a second mixture:
   at least one polysiloxane (b) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H); and
(iii) contacting the second mixture with:
   least one polysiloxane (c) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted.

In certain embodiments, the at least one polysiloxane (a) possesses at a viscosity ranging from about 800 cSt to about 1200 cSt. In certain embodiments, the at least one polysiloxane (a) possesses an average molecular weight ranging from about 15 kDa to about 45 kDa. In certain embodiments, the at least one polysiloxane (a) possesses an average molecular weight of about 28 kDa. In certain embodiments, the at least one polysiloxane (a) possesses an alkenyl equivalent per kilogram selected from the group consisting of 0.01, 0.05, 0.10, 0.15, 0.20, 0.25, 0.30, 0.35, 0.40, 0.45, 0.50, 0.55, 0.60, 0.65, and 0.70. In certain embodiments, the at least one polysiloxane (a) possesses a vinyl equivalent per kilogram ranging from about 0.11 to about 0.15. In certain embodiments, the at least one polysiloxane (a) possesses a vinyl equivalent per kilogram ranging from about 0.5 to about 0.7.

In certain embodiments, the at least one polysiloxane (b) possesses a viscosity of about 10,000 cSt. In certain embodiments, the at least one polysiloxane (b) possesses an average molecular weight ranging from about 45 kDa to about 75 kDa. In certain embodiments, the at least one polysiloxane (b) possesses an average molecular weight of about 62.7 kDa. In certain embodiments, the at least one polysiloxane (b) possesses a hydride weight percent selected from the group consisting of 0.001%, 0.002%, 0.003%, 0.004%, 0.005%, 0.006%, 0.007%, 0.008%, 0.009%, and 0.010%. In certain embodiments, the at least one polysiloxane (b) possesses a hydride weight percent of about 0.003%.

In certain embodiments, the at least one polysiloxane (b) possesses at a viscosity ranging from about 7 cSt to about 10 cSt. In certain embodiments, the at least one polysiloxane (a) possesses an average molecular weight ranging from about 1 kDa to about 1.1 kDa. In certain embodiments, the at least one polysiloxane (a) possesses a hydride weight percentage of about 0.18%, 0.19%, or about 0.20%.

In certain embodiments, the at least one polysiloxane (c) possesses a viscosity ranging from about 5,000 cSt to about 170,000 cSt. In certain embodiments, the at least one polysiloxane (c) possesses a vinyl equivalent per kilogram selected from the group consisting of about 0.010, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.030, 0.031, 0.032, 0.033, 0.034, 0.035, 0.036, 0.037, 0.038, 0.039, 0.040.

In certain embodiments, the at least one polysiloxane (f) possesses a viscosity ranging from about 150 cSt to about 250 cSt. In certain embodiments, the at least one polysiloxane (f) possesses an average molecular weight ranging from about 5 kDa to about 15 kDa. In certain embodiments, the at least one polysiloxane (f) possesses a weight fraction ranging from about 0.01% to about 75% of the reactant composition. In certain embodiments, the at least one polysiloxane (f) possesses a weight fraction selected from the group consisting of about 0.01%, 0.05%, 0.10%, 0.50%, 1.0%, 2.0%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0%, 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, and 75.0%.

In certain embodiments, the at least one polysiloxane (a) is a compound of formula (Ia):

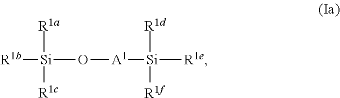

wherein:
A¹ comprises m units of

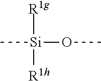

monomer and n units of

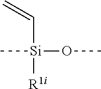

monomer,
   wherein each ---- bond is a Si—O bond;
m is an integer ranging from 410 to 470;
n is an integer ranging from 1 to 50;
R^{1a}, R^{1b}, R^{1c}, R^{1d}, R^{1e}, and R^{1f} are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, optionally substituted C₂-C₁₂ heteroaryl, and optionally substituted C₂-C₆ alkenyl,
   wherein R^{1a}, R^{1b}, R^{1c}, R^{1d}, R^{1e}, and R^{1f} are selected such that each Si atom is substituted with no more than one C₂-C₆ alkenyl; and
R^{1g}, R^{1h}, and R¹ⁱ are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl.

In certain embodiments, R^{1a}, R^{1b}, R^{1c}, R^{1d}, R^{1e}, R^{1f}, R^{1g}, R^{1h}, R¹ⁱ, m, and n are selected such that the at least one polysiloxane (a) possesses a viscosity ranging from about 800 cSt to about 1200 cSt. In certain embodiments, R^{1a}, R^{1b}, R^{1c}, R^{1d}, R^{1e}, R^{1f}, R^{1g}, R^{1h}, R¹ⁱ, m, and n are selected such that the at least one polysiloxane (a) possesses an average molecular weight ranging from about 15 kDa to about 45 kDa. In certain embodiments, R^{1a}, R^{1b}, R^{1c}, R^{1d}, R^{1e}, R^{1f}, R^{1g}, R^{1h}, R¹ⁱ, m, and n are selected such that the at least one polysiloxane (a) possesses an average molecular weight of about 28 kDa. In certain embodiments, R^{1a}, R^{1b}, R^{1c}, R^{1d}, R^{1e}, R^{1f}, R^{1g}, R^{1h}, R¹ⁱ, m, and n are selected such that the at least one polysiloxane (a) possesses an alkenyl equivalent per kilogram selected from the group consisting of 0.01, 0.05, 0.10, 0.15, 0.20, 0.25, 0.30, 0.35, 0.40, 0.45, 0.50, 0.55, 0.60, 0.65, and 0.70.

In certain embodiments, the at least one polysiloxane (b) is a compound of formula (Ib):

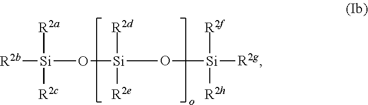

wherein:
R^{2a}, R^{2b}, R^{2c}, R^{2f}, R^{2g}, and R^{2h} are each independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
each occurrence of R^{2d} and R^{2e} is independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
o is an integer ranging from 500 to 1500; and
wherein R^{2a}, R^{2b}, R^{2c}, R^{2d}, R^{2e}, R^{2f}, R^{2g}, and R^{2h} are selected such that each Si atom is substituted with no more than one H atom.

In certain embodiments, R^{2a}, R^{2b}, R^{2c}, R^{2d}, R^{2e}, R^{2f}, R^{2g}, R^{2h}, and o are selected such that the at least one polysiloxane (b) possesses a viscosity of about 10,000 cSt. In certain embodiments, R^{2a}, R^{2b}, R^{2c}, R^{2d}, R^{2e}, R^{2f}, R^{2g}, R^{2h}, and o are selected such that the at least one polysiloxane (b) possesses an average molecular weight ranging from about 45 kDa to about 75 kDa. n certain embodiments, R^{2a}, R^{2b}, R^{2c}, R^{2d}, R^{2e}, R^{2f}, R^{2g}, R^{2h}, and o are selected such that the at least one polysiloxane (b) possesses an average molecular weight of about 62.7 kDa. In certain embodiments, R^{2a}, R^{2b}, R^{2c}, R^{2d}, R^{2e}, R^{2f}, R^{2g}, R^{2h}, and o are selected such that the at least one polysiloxane (b) possesses a hydride weight percent selected from the group consisting of about 0.001%, 0.002%, 0.003%, 0.004%, 0.005%, 0.006%, 0.007%, 0.008%, 0.009%, and about 0.01%.

In certain embodiments, R^{2a}, R^{2b}, R^{2c}, R^{2d}, R^{2e}, R^{2f}, R^{2g}, R^{2h}, and o are selected such that the at least one polysiloxane (b) possesses a viscosity of about 7 cSt, 8 cSt, 9 cSt, or about 10 cSt. In certain embodiments, R^{2a}, R^{2b}, R^{2c}, R^{2d}, R^{2e}, R^{2f}, R^{2g}, R^{2h}, and o are selected such that the at least one polysiloxane (b) possesses an average molecular weight ranging from about 1.0 kDa to about 1.1 kDa. In certain embodiments, R^{2a}, R^{2b}, R^{2c}, R^{2d}, R^{2e}, R^{2f}, R^{2g}, R^{2h}, and o are selected such that the at least one polysiloxane (b) possesses an average molecular weight of about 62.7 kDa. In certain embodiments, R^{2a}, R^{2b}, R^{2c}, R^{2d}, R^{2e}, R^{2f}, R^{2g}, R^{2h}, and o are selected such that the at least one polysiloxane (b) possesses a hydride weight percent selected from the group consisting of about 0.18, 0.19, or about 0.20%.

In certain embodiments, the at least one polysiloxane (c) is a compound of formula (Ic):

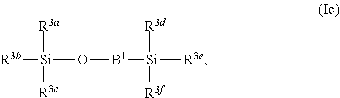

wherein:
B¹ comprises p units of

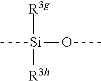

monomer and q units of

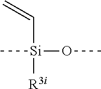

monomer,
   wherein each ---- bond is a Si—O bond;
R^{3a}, R^{3b}, R^{3c}, R^{3d}, R^{3e}, and R^{3f} are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, optionally substituted C₂-C₁₂ heteroaryl, and optionally substituted C₂-C₆ alkenyl,
   wherein R^{3a}, R^{3b}, R^{3c}, R^{3d}, R^{3e}, and R^{3f} are selected such that each Si atom is substituted with no more than one optionally substituted C₂-C₆ alkenyl;
R^{3g}, R^{3h}, and R³ⁱ are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
p is an integer ranging from 500 to 2000; and
q is an integer ranging from 0 to 50.

In certain embodiments, R^{3a}, R^{3b}, R^{3c}, R^{3d}, R^{3e}, R^{3f}, p, and q, are selected such that the at least one polysiloxane (c) possesses a viscosity ranging from about 5,000 cSt to about 170,000 cSt. In certain embodiments, R^{3a}, R^{3b}, R^{3c}, R^{3d}, R^{3e}, R^{3f}, p, and q, are selected such that the at least one polysiloxane (c) possesses an alkenyl equivalent per kilogram selected from the group consisting of about 0.010, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.030, 0.031, 0.032, 0.033, 0.034, 0.035, 0.036, 0.037, 0.038, 0.039, 0.040.

In certain embodiments, the at least one polysiloxane (f) is a compound of formula (If):

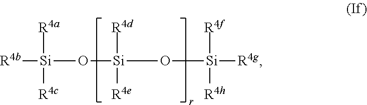

wherein:
R^{4a}, R^{4b}, R^{4c}, R^{4f}, and R^{4g} are each independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
each occurrence of R^{4d} and R^{4e} is independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
r is an integer ranging from 100 to 400; and
wherein no more than one of R^{4a}, R^{4b}, R^{4c}, R^{4d}, R^{4e}, R^{4f}, R^{4g}, and R^{4h} is H.

In certain embodiments, R^{4a}, R^{4b}, R^{4c}, R^{4d}, R^{4e}, R^{4f}, R^{4g}, R^{4h}, and r are selected such that the at least one polysiloxane (f) possesses a viscosity ranging from about 150 cSt to about 250 cSt. In certain embodiments, R^{4a}, R^{4b}, R^{4c}, R^{4d}, R^{4e}, R^{4f}, R^{4g}, R^{4h}, and r are selected such that the at least one polysiloxane (f) possesses an average molecular weight ranging from about 5 kDa to about 15 kDa. In certain embodiments, R^{4a}, R^{4b}, R^{4c}, R^{4d}, R^{4e}, R^{4f}, R^{4g}, R^{4h}, and r are selected such that the at least one polysiloxane (f) possesses an average molecular weight of about 10 kDa. In certain embodiments, R^{4a}, R^{4b}, R^{4c}, R^{4d}, R^{4e}, R^{4f}, R^{4g}, R^{4h}, and r are selected such that the at least one polysiloxane (f) possesses a weight fraction ranging from about 0.01% to about 75% of the reactant composition.

In certain embodiments, the Group X catalyst comprises Pt. In certain embodiments, the Pt is Pt(0). In certain embodiments, the Group X catalyst is Karstedt's catalyst:

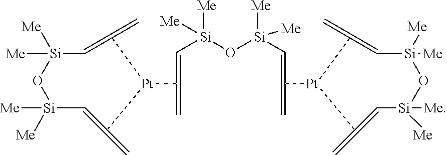

In certain embodiments, the inert formulation-compatible polysiloxane is a compound of formula (Id):

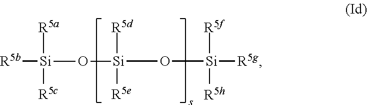

wherein:
R^{5a}, R^{5b}, R^{5c}, R^{5d}, R^{5e}, R^{5f}, R^{5g}, and R^{5h} are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl; and
s is an integer ranging from 1 to about 500.

In certain embodiments, the inert formulation-compatible polysiloxane is selected from the group consisting of polydimethylsiloxane, dimethiconol, disiloxane, trisiloxane, and diphenyl dimethicone/vinyl diphenyl dimethicone/silsesquioxane cross-polymer.

In certain embodiments, the inert formulation-compatible polysiloxane is decamethylcyclopentasiloxane.

In certain embodiments, R^{1a}, R^{1b}, R^{1c}, R^{1d}, R^{1e}, R^{1f}, R^{1g}, R^{1h}, and R¹ⁱ are each independently CH₃ or CH═CH₂. In certain embodiments, R^{2a}, R^{2b}, R^{2c}, R^{2d}, R^{2e}, R^{2f}, R^{2g}, and R^{2h} are each independently H or CH₃. In certain embodiments, R^{3a}, R^{3b}, R^{3c}, R^{3d}, R^{3e}, and R^{3f} are each independently CH₃ or CH═CH₂. In certain embodiments, R^{3g}, R^{3h}, and R³ⁱ are each independently CH₃ or CH═CH₂. In certain embodiments, R^{4a}, R^{4b}, R^{4c}, R^{4d}, R^{4c}, R^{4f}, R^{4g}, and R^{4h} are each independently H or CH₃. In certain embodiments, R^{5a}, R^{5b}, R^{5c}, R^{5d}, R^{5e}, R^{5f}, R^{5g}, and R^{5h} are each independently CH₃.

In certain embodiments, the composition further comprises at least one additive.

In certain embodiments, additive is at least one selected from the group consisting of glycerin, cetyl diglyceryl tris(trimethylsiloxy)silylethyl dimethicone, hexamethyldisilazane (HMDS) fumed silica, and polyoxyethylene/polyoxypropylene copolymer (PEG/PPG-18/18 dimethicone).

In certain embodiments, the composition has a viscosity selected from the group consisting of about 4000, 5000, 6000, 7000, 8000, 9000, 10000, 15000, 20000, 25000, 30000, 35000, 40000, 45000, 50000, 55000, 60000, 65000, 70000, 75000, 80000, 85000, 90000, 95000 and about 100000 cSt.

In certain embodiments, the composition has a total ratio of units of silicon hydride (i.e., Si—H) to vinyl-substituted silicon (i.e., Si—C(H)═CH₂) in all reactant components selected from the group consisting of 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, and about 0.80.

In certain embodiments, the at least one polysiloxane (a) comprises trimethylsiloxy terminated, 0.8-1.2% vinylmethylsiloxane dimethylsiloxane copolymer. In certain embodiments, the at least one polysiloxane (b) comprises hydride terminated polydimethylsiloxane. In certain embodiments, the at least one polysiloxane (c) comprises vinyl terminated poly dimethylpolysiloxane. In certain embodiments, the at least one inert formulation-compatible polysiloxane comprises polydimethy siloxane and/or decamethylcyclopentasiloxane. In certain embodiments, the at least one Group X transition metal catalyst comprises Karstedt's catalyst. In certain embodiments, the at least one additional polysiloxane (f) comprises monohydride terminated polydimethylpolysiloxane.

In certain embodiments, the at least one polysiloxane (a) comprises about 14.0% to about 20.0% of the composition by weight (w/w %). In certain embodiments, the at least one polysiloxane (b) comprises about 0.20% to about 0.40% of the composition by weight (w/w %). In certain embodiments, the at least one polysiloxane (c) comprises about 18% to about 40% of the composition by weight (w/w %). In certain embodiments, the at least one formulation-compatible polysiloxane comprises about 35% to about 50% of the composition by weight (w/w %). In certain embodiments, the at least one Group X catalyst comprises about 0.0080% (80 ppm) to about 0.0120% (120 pm) of the composition by weight (w/w %). In certain embodiments, the at least one additional polysiloxane (f) comprises about 7% to about 12% of the composition by weight (w/w %).

In certain embodiments, the at least one polysiloxane (a) comprises about 15.5% of the composition by weight (w/w %). In certain embodiments, the at least one polysiloxane (b) comprises about 0.30% of the composition by weight (w/w %). In certain embodiments, the at least one polysiloxane (c) comprises about 35.5% of the composition by weight (w/w %). In certain embodiments, the at least one formulation-compatible polysiloxane comprises about 37.3% of the composition by weight (w/w %). In certain embodiments, the at least one Group X catalyst comprises about 0.0090% (90 ppm) of the composition by weight (w/w %). In certain embodiments, the at least one additional polysiloxane (f) comprises about 8.5% of the composition by weight (w/w %).

In certain embodiments, the at least one polysiloxane (a) comprises about 18.9% of the composition by weight (w/w %). In certain embodiments, the at least one polysiloxane (b) comprises about 0.30% of the composition by weight (w/w %). In certain embodiments, the at least one polysiloxane (c) comprises about 21.60% of the composition by weight (w/w %). In certain embodiments, the at least one formulation-compatible polysiloxane comprises about 45.1% of the composition by weight (w/w %). In certain embodiments, the at least one Group X catalyst comprises about 0.0108% (108 ppm) of the composition by weight (w/w %). In certain embodiments, the at least one additional polysiloxane (f) comprises about 10.4% of the composition by weight (w/w %).

In certain embodiments, the at least one polysiloxane (a) comprises about 6.0% to about 12.0% of the composition by weight (w/w %). In certain embodiments, the at least one polysiloxane (b) comprises about 6.0% to about 12.0% of the composition by weight (w/w %). In certain embodiments, the at least one polysiloxane (c) comprises about 20.0% to about 30.0% of the composition by weight (w/w %). In certain embodiments, the at least one formulation-compatible polysiloxane comprises about 10.0% to about 60.0% of the composition by weight (w/w %). In certain embodiments, the at least one Group X catalyst comprises about 0.001% (10 ppm) to about 0.02% (200 ppm) of the composition by weight (w/w %). In certain embodiments, the at least one additional polysiloxane (f) comprises about 1.0% to about 10.0% of the composition by weight (w/w %).

In certain embodiments, the at least one polysiloxane (a) comprises about 9.0% of the composition by weight (w/w %). In certain embodiments, the at least one polysiloxane (b) comprises about 9.4% of the composition by weight (w/w %). In certain embodiments, the at least one polysiloxane (c) comprises about 24.2% of the composition by weight (w/w %). In certain embodiments, the at least one formulation-compatible polysiloxane comprises about 49% of the composition by weight (w/w %). In certain embodiments, the at least one Group X catalyst comprises about 0.0048% (48 ppm) of the composition by weight (w/w %). In certain embodiments, the at least one additional polysiloxane (f) comprises about 5.8% of the composition by weight (w/w %).

In certain embodiments, the at least one polysiloxane (a) comprises about 17.18% of the composition by weight (w/w %). In certain embodiments, the at least one polysiloxane (b) comprises about 0.26% of the composition by weight (w/w %). In certain embodiments, the at least one polysiloxane (c) comprises about 19.69% of the composition by weight (w/w %). In certain embodiments, the at least one formulation-compatible polysiloxane comprises about 44.00% of the composition by weight (w/w %). In certain embodiments, the at least one Group X catalyst comprises about 0.0099% (99 ppm) of the composition by weight (w/w %). In certain embodiments, the at least one additional polysiloxane (f) comprises about 9.45% of the composition by weight (w/w %).

In certain embodiments, each occurrence of optionally substituted alkyl, optionally substituted cycloalkyl, optionally substituted alkenyl, optionally substituted heteroalkyl, optionally substituted heterocycloalkyl, optionally substituted benzyl, optionally substituted aryl, optionally substituted heteroaryl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

### Multilayer Composition Application

In another aspect, the present disclosure provides a method for applying a multilayered wound dressing composition to a wound of a subject, the method comprising:
(a) applying to the surface of the wound an adhesive basal layer comprising the composition of the present disclosure, wherein the adhesive basal layer optionally further comprises one or more additional compounds and/or compositions selected from the group consisting of a non-reactive fluid, emulsifier, and a pharmaceutically active agent and/or composition; and
(b) applying to the surface of the adhesive basal layer a mechanically reinforcing layer comprising the composition of the present disclosure;
   wherein the adhesive basal layer is in contiguous contact with at least a portion of the surface of the wound; and
   wherein the mechanically reinforcing layer is in contiguous contact with at least a portion of the surface of the adhesive basal layer.

In certain embodiments, the adhesive basal layer is applied with a roller. In certain embodiments, the roller is metal.

In certain embodiments, the adhesive basal layer has a thickness of about 10 μm to about 100 μm.

In certain embodiments, the mechanically reinforcing layer is applied with an applicator tool. In certain embodiments, the applicator tool is metal. In certain embodiments, the applicator is a roller.

In certain embodiments, at least one of the adhesive basal layer and the mechanically reinforcing layer have a uniform thickness.

In certain embodiments, the wound is caused by mechanical shearing and/or puncturing of the skin of the subject.

In certain embodiments, the wound is caused by a skin condition.

In certain embodiments, the skin condition is at least one of xerosis, ichthyosis, eczema, contact dermatitis, diaper rash, radiation dermatitis, and psoriasis.

In certain embodiments, the adhesive basal layer is applied to a wound which has been treated and/or coated with one or more topically active compounds and/or compositions.

In another aspect, the present disclosure provides a method of treating a skin condition and/or wound of a subject, the method comprising:
(a) applying to the surface of the wound an adhesive basal layer comprising the composition of the present disclosure, wherein the adhesive basal layer optionally further comprises one or more additional compounds and/or compositions selected from the group consisting of a non-reactive fluid, emulsifier, a cosmetic, and a pharmaceutically active agent and/or composition; and
(b) applying to the surface of the adhesive basal layer a mechanically reinforcing layer comprising the composition of the present disclosure;
   wherein the adhesive basal layer is in contiguous contact with at least a portion of the surface of the wound; and
   wherein the mechanically reinforcing layer is in contiguous contact with at least a portion of the surface of the adhesive basal layer.

In certain embodiments, the adhesive basal layer is applied with a roller. In certain embodiments, the roller is metal.

In certain embodiments, the adhesive basal layer has a thickness of about 10 μm to about 100 μm.

In certain embodiments, the mechanically reinforcing layer is applied with an applicator tool. In certain embodiments, the applicator tool is metal. In certain embodiments, the applicator is a roller.

In certain embodiments, at least one of the adhesive basal layer and the mechanically reinforcing layer have a uniform thickness.

In certain embodiments, the wound is caused by mechanical shearing and/or puncturing of the skin of the subject.

In certain embodiments, the wound is caused by a skin condition.

In certain embodiments, the skin condition is selected from the group consisting of xerosis, ichthyosis, eczema, contact dermatitis, diaper rash, radiation dermatitis, and psoriasis.

In certain embodiments, the adhesive basal layer is applied to a wound which has been treated and/or coated with one or more topically active compounds and/or compositions.

### Kits

In another aspect, the present disclosure provides a kit comprising:
(a) a container comprising the telechelic polymer composition of the present disclosure, wherein the container is suitable for dispensation;
(b) a container comprising the mechanically reinforcing composition (MRC) of the present disclosure, wherein the container is suitable for dispensation; and
(c) instructional materials for use thereof.

In certain embodiments, the kit further comprises a roller. In certain embodiments, the roller is a metal roller.

In certain embodiments, the kit further comprises an applicator. In certain embodiments, the applicator is a metal applicator. In certain embodiments, the applicator is a roller. In certain embodiments, the kit further comprises applicator maintenance wipes.

### EXAMPLES

Various embodiments of the present application can be better understood by reference to the following Examples which are offered by way of illustration. The scope of the present application is not limited to the Examples given herein.

### Example 1: F3-99-2 Telechelic Composition

The crosslinked organosilicon telechelic composition F3-99-2 is shown in Table 1. The hydride to vinyl ratio for Component 1B to Component TA is maintained at 1:1. The vinyl component 2 is introduced in excess at 2 times the vinyl content of component TA. The total vinyl to the total hydride ratio ([H]/[V]) is 0.3.

Component 1 (i.e., F3-99-1) comprises components 1A, 1B, 1C and 1D. Component 1D (F3-28-1) was made by diluting 1 part 2% Pt Karstedt's Catalyst (Dong Chen Chemistry Material, Guang Dong, China) into 9 parts decamethylcyclopentasiloxane to make the 2000 ppm Pt catalyst.

F3-99-1 was prepared by first mixing Components 1A and 1B in a cup (cup A) for 30 seconds using a planetary centrifugal mixer (THINKY AR-100, Laguna Hills, CA). Similarly, Components 1C and 1D were mixed in a cup (cup B) for 30 seconds. The contents of cup A and cup B (Component 1) were then mixed by hand using a spatula until the mixture was uniform in appearance. A chronometer was used to measure the total reaction time (tᵣₓₙ), where tᵣₓₙ=0 minutes corresponds to the introduction of the metal catalyst to the starting components.

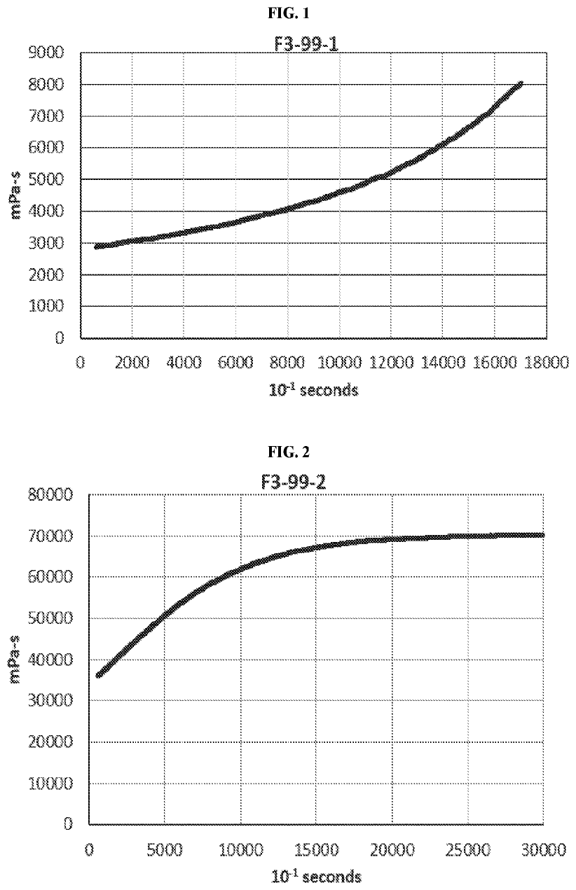

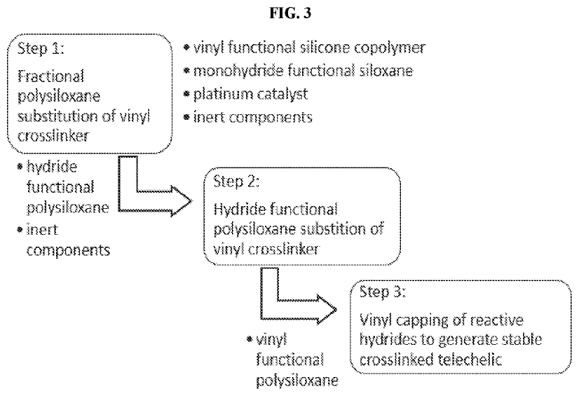

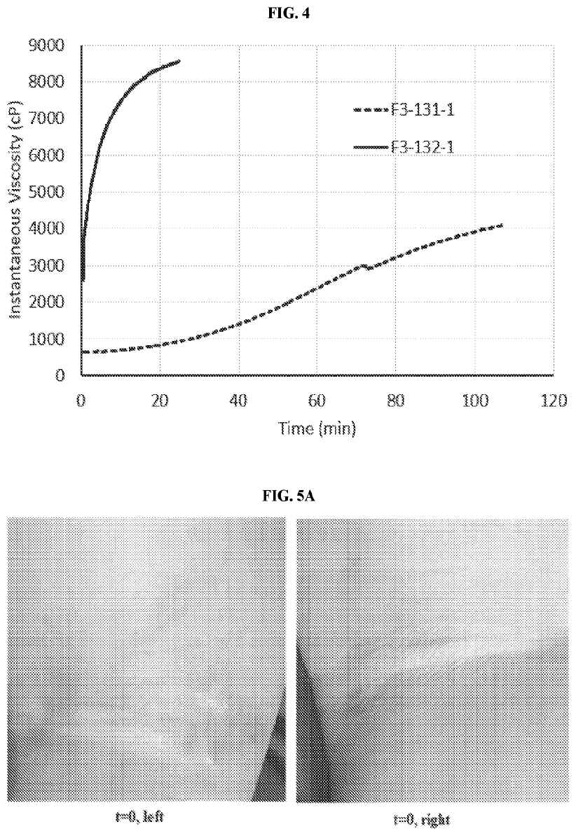

Starting at tᵣₓₙ=4.5 minutes, Component 1 was continuously monitored at 25° C. with a viscometer (Spindle 4, 20 RPM, IKA lo-vi heli, Wilmington, NC) over the first phase of the crosslinking reaction, where the formulation samples were transferred to 4 dram amber glass vials. FIG. 1 shows the instantaneous viscosity as a function of reaction time, where t=0 minutes corresponds to tᵣₓₙ=4.5 minutes.

When Component 1 achieved a viscosity of 8000 mPa-s, Component 2 was introduced (tᵣₓₙ=34 minutes) and mixed for 5 minutes using a Jiffy mixing blade at 225 RPM (IKA RW20). The viscosity of F3-99-2 was continuously monitored, until a plateau was reached at 70,000 mPa-s. The instantaneous viscosity is plotted in FIG. 2, where t=0 minutes represents tᵣₓₙ=46 minutes.

TABLE 1
F3-99-2 cross-linked telechelic composition

| | | | | Mass | w/w |
|---|---|---|---|---|---|
| No. | Chemical | Ref. | mmol/g | (g) | (%) |
| 1 | F3-99-1 (STA) | — | | 61.9 | 58 |
| 1A | 0.8-1.2% vinylmethylsiloxane | V1 | 0.13 | 6.8 | 6 |
| | dimethylsiloxane copolymer, | | | | |
| | trimethylsiloxy terminated, | | | | |
| | 800-1200 cSt (VDT-131, | | | | |
| | Gelest) | | | | |
| 1B | hydride terminated | H1 | 0.031 | 28.5 | 27 |
| | polydimethylsiloxane, | | | | |
| | 10,000 cSt (DMS H-41, | | | | |
| | Gelest) | | | | |
| 1C | polydimethyl siloxane 95-105 | — | — | 25.1 | 23 |
| | mPa · s (Belsil DM100, | | | | |
| | Wacker) | | | | |
| 1D | 2000 ppm Karstedt's | — | — | 1.5 | 1 |
| | catalyst in KF995 | | | | |
| | (F3-28-1, STA) | | | | |
| 2 | vinyl terminated | V2 | 0.04 | 45.5 | 42 |
| | dimethylpolysiloxane, | | | | |
| | 20,000 cSt (VS 20000, | | | | |
| | Andisil) | | | | |

| | |
|---|---|
| [H]/[V] | 0.3 |
| [H1]/[V1] | 1.00 |

### Example 2: F3-101-2 Adhesive Base Composition

The F3-101-2 Adhesive Base Composition is shown in Table 2. Component 2 was added to freshly prepared Component 1, which is comprised of Components 1A, 1B, 1C, 1D and 1E, and mixed for 5 minutes at 225 RPM. Components 3 and 4 ere then added and mixed for 5 minutes at 175 RPM. Finally, Component 5 was added and mixed for 5 minutes at a speed of 250 RPM. A Jiffy mixing blade attached to an IKA overhead mixer (model RW20) was used for the Adhesive Base Composition mixing steps. The F3-101-2 duplicate viscosity measurements taken on the following day were 9059 mPa·s and 9299 mPa·s. The viscosity was measured using Spindle 4 at 10 RPM for 2 minutes (IKA lo-vi viscometer).

TABLE 2
F3-101-2 Adhesive base composition

| | | | | Mass | w/w |
|---|---|---|---|---|---|
| No. | Chemical | Ref. | mmol/g | (g) | (%) |
| 1 | F3-99-1 (STA) | — | | 25.4 | 48% |
| 1A | 0.8-1.2% vinylmethylsiloxane | V1 | 0.13 | 1.6 | 3% |
| | dimethylsiloxane copolymer, | | | | |
| | trimethylsiloxy terminated, | | | | |
| | 800-1200 cSt (VDT-131, | | | | |
| | Gelest) | | | | |
| 1B | hydride terminated | H1 | 0.031 | 6.7 | 13% |
| | polydimethylsiloxane, | | | | |
| | 10,000 cSt (DMS H-41, | | | | |
| | Gelest) | | | | |
| 1C | polydimethyl siloxane | — | | 5.9 | 11% |
| | 95-105 mPa · s | | | | |
| | (Belsil DM100, Wacker) | | | | |
| 1D | 2000 ppm Karstedt's | — | | 0.4 | 1% |
| | catalyst in KF995 | | | | |
| | (F3-28-1, STA) | | | | |
| 1E | vinyl terminated | V2 | 0.04 | 10.8 | 20% |
| | dimethylpolysiloxane, | | | | |
| | 20,000 cSt (VS 20000, | | | | |
| | Andisil) | | | | |
| 2 | vinyl terminated | | 0.015 | 4.6 | 9% |
| | dimethylpolysiloxane, | | | | |
| | 20,000 cSt (VS 20000, | | | | |
| | Andisil) | | | | |
| 3 | Dimethiconol (PMX-1501, Dow) | | | 9.2 | 17% |
| 4 | 2000 ppm Karstedt's | | | 0.8 | 1% |
| | Catalyst in KF-995 | | | | |
| | (F3-99-1-2) | | | | |
| 5 | Disiloxane (Xiameter | | | 13.3 | 25% |
| | PMX-200, 0.65 cSt) | | | | |

| | |
|---|---|
| [H]/[V] | 0.29 |
| [H1]/[V1] | 1.00 |
| [V2]/[V1] | 2.07 |
| [V3]/[V2] | 0.16 |

### Example 3: Adhesive Base Formulation Variant Screening

Tables 3a-3b show the compositions of the Adhesive Bases derived from F3-101-2. For each composition, an emulsifier (Component 2 or Component 3) was mixed with the F3-101-2 Adhesive Base for 5 minutes at 250 RPM. With the mixing blade at a speed of 250 RPM, the corresponding quantity of glycerin was then added dropwise to the formulation and mixed for 5 additional minutes.

The duplicate viscosity and torque (Tau) measurements that were taken on the following day are also reported for each formulation. Despite demonstrating the best visual formulation emulsion stability, the addition of 8% Component 3 emulsifier for F3-102-2 resulted in a viscosity that was 1.5 times higher than the viscosity of F3-102-1. This increase in viscosity may approach the higher range of commercially feasible formulations as the application to the skin may be limited by the spreadability of viscous materials.

TABLE 3a
Adhesive base compositions comprising cross-linked telechelics

| Component No. | Chemical |
|---|---|
| 1 | F3-101-2, STA |
| 2 | Cetyl Diglyceryl Tris(Trimethylsiloxy) |
| | silylethyl Dimethicone (Dowsil ES5600, Dow) |
| 3 | Cyclopentasiloxane (and) PEG/PPG-19/19 |
| | Dimethicone (Dowsil BY11-030, Dow) |
| 4 | Glycerin (glycerin 99.7%, Acme Hardesty) |

TABLE 3b
Adhesive base compositions comprising cross-linked telechelics

| | F3-101-3 | | F3-101-4 | | F3-102-1 | | F3-102-2 | |
|---|---|---|---|---|---|---|---|---|
| No. | grams | w/w | grams | w/w | grams | w/w | grams | w/w |
| 1 | 20 | 97% | 20 | 95% | 20 | 97% | 20 | 95% |
| 2 | 0.22 | 1% | 0.65 | 3% | 0 | 0% | 0 | 0% |
| 3 | 0 | 0% | 0 | 0% | 0.8 | 4% | 1.8 | 8% |
| 4 | 0.4 | 2% | 0.4 | 2% | 0.4 | 2% | 0.4 | 2% |
| Viscosity | 13198 | 14458 | 11879 | 12958 | 13198 | 14458 | 11879 | 12958 |
| (mPa · s) | | | | | | | | |
| Tau (%) | 22 | 24.1 | 19.8 | 21.6 | 22 | 24.1 | 19.8 | 21.6 |

### Example 4: Adhesive Base Formulation Stability at 40° C.

The AB compositions in Example 3 were placed on stability in a 40° C. oven. Duplicate viscosity and torque measurements were made in 4 dram amber glass vials at each time point (Tables 4a-4b; IKA Rotovisc Lo-Vi viscometer, with Spindle Number 4 at 10 RPM and a 2 minute run time).

F3-102-2 remained uniform at the 18-week timepoint, whereas the other formulations revealed the presence of sediment.

TABLE 4a
Adhesive base stability at 40° C.

| | CAL-12500 (RT) | | F3-101-2 (40° C.) | | F3-101-3 (40° C.) | |
|---|---|---|---|---|---|---|
| Average viscosity | 13888 | 23% | 9179 | 15% | 13828 | 23% |
| (mPa-s, %)^{a} | | | | | | |
| Viscosity (mPa-s)^{a} | 13858 | 13918 | 9059 | 9299 | 13198 | 14458 |
| Tau (%)^{b} | 23 | 23 | 15 | 16 | 22 | 24 |
| Average viscosity (mPa-s, | 13168 | 22% | 8879 | 15% | 12989 | 22% |
| %)^{b} | | | | | | |
| Viscosity (mPa-s)^{b} | 13078 | 13258 | 9119 | 8639 | 12419 | 13558 |
| Tau (%)^{b} | 22 | 22 | 15 | 14 | 21 | 23 |
| Average viscosity (mPa-s, | 13168 | 22% | 9359 | 16% | 15328 | 26% |
| %)^{c} | | | | | | |
| Viscosity (mPa-s)^{c} | 12718 | 13618 | 9479 | 9239 | 15118 | 15538 |
| Tau (%)^{c} | 21 | 23 | 16 | 15 | 25 | 26 |

^{a}t = 0;
^{b}t = 5 weeks;
^{c}t = 14 weeks.

TABLE 4b
Adhesive base stability at 40° C.

| | F3-101-4 (40° C.) | | F3-102-1 (40° C.) | | F3-102-2 (40° C.) | |
|---|---|---|---|---|---|---|
| Average viscosity (mPa-s, | 12419 | 21% | 13183 | 22% | 20158 | 34% |
| %)^{a} | | | | | | |
| Viscosity (mPa-s)^{a} | 11879 | 12958 | 12778 | 13588 | 20638 | 19678 |
| Tau (%)^{b} | 20 | 22 | 21 | 23 | 34 | 33 |
| Average viscosity (mPa-s, | 12059 | 20% | 13138 | 22% | 20628 | 34% |
| %)^{b} | | | | | | |
| Viscosity (mPa-s)^{b} | 12538 | 11579 | 13558 | 12718 | 20937 | 20318 |
| Tau (%)^{b} | 21 | 19 | 23 | 21 | 35 | 34 |
| Average viscosity (mPa-s, | 12149 | 20% | 13798 | 23 | 21528 | 36% |
| %)^{c} | | | | | | |
| Viscosity (mPa-s)^{c} | 11819 | 12478 | 13618 | 13978 | 21777 | 21279 |
| Tau (%)^{c} | 20 | 21 | 23 | 23 | 36 | 36 |

^{a}t = 0;
^{b}t = 5 weeks;
^{c}t = 14 weeks.

### Example 5: Preparation of Adhesive Base Compositions Comprising Brush Architecture

Tables 5a-5b summarize adhesive base compositions comprising monohydride functional siloxanes with hydride content reflecting 75% substitution of the available vinyl groups present on the vinyl methyl siloxane, dimethylsiloxane copolymer (F3-126-0). Unless otherwise noted, each mixing step was conducted using a centrifugal planetary mixer (AR-100, THINKY Corporation) for 20 seconds.

### Preparation of F3-126-0

F3-126-0 assessed the feasibility and performance of the vinyl functional crosslinked telechelic with 75% monohydride siloxane substitution of the vinyl copolymer starting reactant. The Step 1 product was prepared by mixing components (1), (2) and (5), followed by introduction of component (6). The Step 1 product was stored in a bead bath that and maintained at 26 degrees C. After 20 hours, the Step 2 product was synthesized by adding component (3) to the Step 1 product. 1 minute after mixing the components, the formulation viscosity increased substantially, by visual examination, resembling that for a gel. Due to the increase in viscosity beyond the apparent gel point, the addition of component (4) was not assessed, as the application of the crosslinked, viscous gel to the skin was deemed commercially impractical.

### Preparation of F3-134-1 and F3-136-1

For F3-134-1 and F3-136-1, Step 1 comprised the reaction of components (1), (2), (5) and (6) until a stable viscosity was achieved. In process Step 2, components (3) and (7) were then introduced to the product generated in Step 1. The Step 2 instantaneous viscosity was monitored to establish a target viscosity window for the Step 3 addition of excess vinyl siloxane (4). The vinyl and hydride ratios corresponding to the compositions in Table 5b are shown in Table 6. The viscosity measurements recorded for the reaction steps corresponding to each formulation is reported in Table 7.

TABLE 5a
F3-126-0, F3-134-1, F3-133-1, and F3-136-1 compositions

| | | | mmol/g vinyl or |
|---|---|---|---|
| No. | Chemical | Ref. | hydride |
| 1 | 0.8-1.2% vinylmethylsiloxane | V1 | 0.13 |
| | dimethylsiloxane copolymer, | | |
| | trimethylsiloxy terminated, | | |
| | 800-1200 cSt (VDT-131, Gelest) | | |
| 2 | monohydride terminated | H1 | 0.1 |
| | polydimethylsiloxane, | | |
| | asymmetric, 150-250 cSt | | |
| | (MCR-H22, Gelest) | | |
| 3 | hydride terminated | H2 | 0.031 |
| | polydimethylsiloxane, 10,000 | | |
| | cSt (DMS H-41, Gelest) | | |
| 4 | vinyl terminated | V2 | 0.04 |
| | dimethylpolysiloxane, 20,000 | | |
| | cSt (VS20000, AB Specialty | | |
| | Silicones) | | |
| 5 | polydimethyl siloxane 95-105 | — | NA |
| | mPa-s (Belsil DM100, Wacker) | | |
| 6 | 2000 ppm Karstedt's Catalyst | — | NA |
| | (1:9 Umicore HS235: PMX0245) | | |
| | (F3-124-1, STA) | | |
| 7 | Decamethylcyclopentasiloxane | — | NA |
| | (PMX0245, Dow) | | |

TABLE 5b
F3-134-1, F3-133-1, and F3-136-1 compositions

| | F3-126-0 | F3-134-1 | F3-136-1 | F3-126-0 | F3-134-1 | F3-136-1 |
|---|---|---|---|---|---|---|
| No. | Mass (g) | Mass (g) | Mass (g) | w/w (%) | w/w (%) | w/w (%) |
| 1 | 2.6 | 1.7 | 5.1 | 22.5% | 8.4% | 10.8% |
| 2 | 2.6 | 0.5 | 3.4 | 21.9% | 2.7% | 7.1% |
| 3 | 2.7 | 2.6 | 5.7 | 23.2% | 12.9% | 12.1% |
| 4 | 0 | 4.1 | 8.8 | 0.0% | 20.4% | 18.4% |
| 5 | 3.1 | 3.9 | 9 | 26.2% | 19.1% | 18.9% |
| 6 | 0.7 | 0.5 | 1.1 | 6.2% | 2.4% | 2.4% |
| 7 | 0 | 6.9 | 14.4 | 0.0% | 34.2% | 30.3% |

TABLE 6
Vinyl and hydride molar ratios for process confirmation studies

| | F3-126-0 | F3-134-1 | F3-136-1 |
|---|---|---|---|
| [H1/V1] | 0.75 | 0.25 | 0.5 |
| [H2/V1] | 0.25 | 0.37 | 0.27 |
| [V2/H2] | 0 | 2.04 | 1.97 |
| [H/V] | 1 | 0.35 | 0.51 |

TABLE 7
Reaction product viscosity measurements
for process confirmation studies

| Telechelic | Process | Stability | Viscosity | Tau | Temp. | |
|---|---|---|---|---|---|---|
| Formulation | Step | Time | (cP) | (%) | (C.) | RPM |
| F3-134-1 | 1 | 0 | 246 | 8.2 | 26 | 200 |
| F3-134-1 | 1 | 1 | 327 | 10.9 | 25 | 200 |
| F3-134-1 | 1 | 1 | 324 | 10.8 | 25 | 200 |
| F3-134-1 | 2 | 0 | 4092 | 68.2 | 25 | 100 |
| F3-134-1 | 2 | 1 | 5267 | 87.8 | 25 | 100 |
| F3-134-1 | 2 | 1 | 5165 | 86.1 | 25 | 100 |
| F3-134-1 | 3 | 0 | 3588 | 29.9 | 26 | 50 |
| F3-134-1 | 3 | 1 | 3324 | 27.7 | 25 | 50 |
| F3-134-1 | 3 | 1 | 3336 | 27.8 | 25 | 50 |
| F3-136-1 | 2 | 1 | 9815 | 81.8 | 25 | 50 |
| F3-136-1 | 2 | 1 | 9407 | 78.4 | 25 | 50 |
| F3-136-1 | 3 | 0 | 4991 | 41.6 | 26 | 50 |
| F3-136-1 | 3 | 1 | 5195 | 43.3 | 25 | 50 |
| F3-136-1 | 3 | 1 | 5327 | 44.4 | 25 | 50 |

### Example 6: F3-137-1 and F3-137-2

Glycerin, a humectant, was incorporated into F3-134-1 at two concentrations (0.6% and 1.9%) (Table 8).

For both formulations, components (1) and (2) were mixed at 150 RPM for 5 minutes, using an overhead mixer (IKA Eurostar 60) and a Jiffy mixing blade (LM model, Jiffy Mixer Co., Inc). The resulting emulsion formed an opaque gel. Component (3) was then added dropwise at 300 RPM and mixed for 30 minutes. The viscosities for each composition were measured in duplicate. The duplicate viscosity measurements recorded for the siloxane brush architecture AB variants following storage at 40° C. accelerated stability conditions are reported in Table 9.

TABLE 8
F3-137-1 and F3-137-2 compositions

| | | | F3-137-1 | | F3-137-2 | |
|---|---|---|---|---|---|---|
| | | | mass | w/w | mass | w/w |
| No. | Manufacturer | Tradename | (g) | (%) | (g) | (%) |
| 1 | STA | F3-134-1 | 13.74 | 91.3% | 13.74 | 91.3% |
| 2 | Dow | Dowsil BY 11- | 1.22 | 8.1% | 1.22 | 8.1% |
| | Chemicals | 030 | | | | |
| 3 | Acme | Glycerin | 0.09 | 0.6% | 0.09 | 0.6% |
| | Hardesty | | | | | |

TABLE 9
Formulation stability at 40° C.

| Formu- | Stability | Viscosity | Viscosity | Torque | Torque |
|---|---|---|---|---|---|
| lation | Time (days) | 1 (cP) | 2 (cP) | 1 (%) | 2 (%) |
| F3-134-1 | 0 | 3588 | — | 29.9 | — |
| F3-134-1 | 1 | 3324 | 3336 | 27.7 | 27.8 |
| F3-134-1 | 19 | 3336 | 3432 | 27.8 | 28.6 |
| F3-136-1 | 0 | 4991 | — | 41.6 | — |
| F3-136-1 | 1 | 5195 | 5327 | 43.3 | 44.4 |
| F3-136-1 | 19 | 4679 | 4907 | 39 | 40.9 |
| F3-137-1 | 0 | 4092 | 4044 | 34.1 | 33.7 |
| F3-137-1 | 18 | 4607 | 4751 | 38.4 | 39.6 |
| F3-137-2 | 0 | 4427 | 4511 | 36.9 | 37.7 |
| F3-137-2 | 18 | 4403 | 4163 | 36.7 | 34.7 |

### Example 7

The effect of the hydride to vinyl ratio on the mechanical properties of the crosslinked material was evaluated for [H]/[V] values of 5, 2, and 1 (F1-81-1, F1-81-2 and F1-81-3 in Tables 10a-10b, respectively). The formulations were prepared using the methods described previously. Each of these formulations immediately cured following catalyst introduction and 15 seconds of mixing.

After 15 minutes of initiating the crosslinking reaction, the samples were manually evaluated. The most stiff material corresponded to F1-81-3, while the softest material was F1-81-1, which had an [H]/[V] value of 5.

The effect of additives used to increase the material toughness was evaluated in the next three formulations (Table 11) where Components 2, 3 and 4 were mixed into F1-81-3, prior to the addition of Component 3, for 30 seconds to make F1-81-4, F1-81-5 and F1-81-6, respectively. Qualitatively, F1-81-6 showed the best material toughness, whereas F1-81-5 remained fluid after 5 minutes of initiating the crosslinking reaction. Following 3 days of curing, F1-81-6 remained the toughest crosslinked material. F1-81-5 cured but remained soft and tacky. F1-81-4 was stiffer than F1-81-5.

In a separate study, 0.55 grams of Component 3 (Table 11) was added to 2.17 grams of F1-81-1 to generate F1-82-1 with an H/V value of 2.38. Approximately 3 drops (or 0.05 g) of F1-31-1 catalyst were added and mixed for 20 seconds. After 10 minutes, the material cured and showed 300% elongation. Compared to F1-81-1, the F1-82-1 was softer and displayed increased tack to the touch.

TABLE 10a

| No. | Chemical | Ref. | mmol/g |
|---|---|---|---|
| 1 | Pendant Silicon-Hydride Functional polydimethyl siloxane, | H1 | 0.95 |
| | trimethylsiloxy terminated, 100 cSt (Andisil XL-1B, AB | | |
| | Specialty Silicones) | | |
| 2 | Vinyl Terminated Dimethylpolysiloxane, 5000 cSt (Andisil | V1 | 0.065 |
| | VS5000, AB Specialty Silicones) | | |
| 3 | 2000 ppm Karstedt's Catalyst in decamethyl | — | — |
| | cyclopentasiloxane (F1-36-1, STA) | | |

TABLE 10b

| | F1-81-1 | F1-81-2 | F1-81-3 | F1-81-1 | F1-81-2 | F1-81-3 |
|---|---|---|---|---|---|---|
| | Mass | Mass | Mass | w/w | w/w | w/w |
| No. | (g) | (g) | (g) | (%) | (%) | (%) |
| 1 | 1.00 | 0.50 | 0.50 | 23.5% | 11.1% | 5.9% |
| 2 | 3.20 | 3.97 | 7.98 | 75.3% | 87.8% | 93.6% |
| 3 | 0.05 | 0.05 | 0.05 | 1.2% | 1.1% | 0.6% |
| [H]/[V] | 4.6 | 1.8 | 0.9 | | | |
| Rapid | Y | Y | Y | | | |
| cure | | | | | | |
| Elongation | 200% + | 200% | 180% | | | |
| Stiffness | 3 | 2 | 1 | | | |

TABLE 11

| | | F1-81- | F1-81- | F1-81- | F1-81- | F1-81- | F1-81- |
|---|---|---|---|---|---|---|---|
| | | 4 | 5 | 6 | 4 | 5 | 6 |
| | | Mass | Mass | Mass | w/w | w/w | w/w |
| No. | Chemical | (g) | (g) | (g) | (%) | (%) | (%) |
| 1 | MRC composition | 1.6 | 1.6 | 1.6 | 79.2% | 79.2% | 79.2% |
| | (F1-83-1, STA) | | | | | | |
| 2 | Cyclopentasiloxane | 0.4 | — | — | 19.8% | 0.0% | 0.0% |
| | (and) | | | | | | |
| | Trimethylsiloxysilicate | | | | | | |
| | (MQ 50C, AB | | | | | | |
| | Specialty Silicones) | | | | | | |
| 3 | Vinyl Q Resin | — | 0.4 | — | 0.0% | 19.8% | 0.0% |
| | Dispersion, 4500-7000 | | | | | | |
| | cSt (VQM 135, | | | | | | |
| | Gelest) | | | | | | |
| 4 | HMDS treated fumed | — | — | 0.4 | 0.0% | 0.0% | 19.8% |
| | silica (Aerosil R8200, | | | | | | |
| | Evonik) | | | | | | |
| 5 | 2000 ppm Karstedt's | 0.02 | 0.02 | 0.02 | 1.0% | 1.0% | 1.0% |
| | Catalyst in decamethyl | | | | | | |
| | cyclopentasiloxane | | | | | | |
| | (F1-36-1, STA) | | | | | | |
| | | | | <5 min | Y | N | Y |
| | | | | cure | | | |

### Example 8: F3-96-1 Mechanical Reinforcing Component (MRC) and Variants

F3-96-1 was produced using the constituent composition of F3-69-1 as a reference and is shown in Tables 12a-12c. Components 1, 2, 3, 4 and 7 were first mixed together and then Component 6 and Component 5 were sequentially introduced. A cycle of 30 seconds was used for each mixing step using the Thinky AR-100.

Using an overhead mixer, the formulation was then mixed for an additional 30 minutes to fully disperse the particles. Component 8 was then introduced and mixed for 30 minutes. Component 7 was introduced to the formulation to account for the evaporated fraction as a result of the processing. Component 9 was finally introduced and mixed until the composition was uniform.

F3-111-1, F3-112-1 and F3-113-1 (Tables 12a-12c) were produced to examine the effects of modulating the volatile solvent component in F3-96-1. For F3-111-1, Component 6 and then Component 5 and Component 8 (phase A) were sequentially mixed into component 7. Components 1, 2, 3 and 4 (phase B) were mixed in a separate cup. The THINKY AR-100 mixer was used for each mixing period of 30 seconds. Using an overhead mixer, Phase A was added to Phase B and mixed for 30 minutes at 1100 RPM. The evaporated fraction of Component 7 was calculated and mixed into the formulation. F3-112-1 and F3-113-1 were produced using a similar process, where Phase A comprised the filler and the volatile component.

These compositions were further modified to assess the benefit of increasing the filler particle loading (Component 4 in Table 13) to make F3-115-1, F3-115-2 and F3-115-3. For each formulation, Component 4 was mixed for 20 seconds (THINKY AR-100). The resulting formulations were applied to the forearms of a volunteer following the scheme shown in Table 14. One full pump of AB and one full pump of the MRC were dispensed onto a metal roller and a metal spatula, respectively. The AB was applied to the designated skin site and the MRC was then applied to the AB to generate the adhesive dressing. Following 24 hours of wear, greater than 90% of the dressings was present at sites 1, 2, 3 and 6. Sites 4 and 5 had closer to 80% of dressing remaining. The dressing at sites 3 and 4 appeared more wrinkled compared to the other sites, which is consistent with the use of Component 9 in previous formulation optimization work.

TABLE 12a
F3-96-1 and volatile component variants

| No. | Chemical | Ref. | mmol/g |
|---|---|---|---|
| 1 | Pendant Silicon-Hydride Functional polydimethyl | H1 | 4.35 |
| | siloxane, trimethylsiloxy terminated (Andisil XL-11, AB | | |
| | Specialty Silicones) | | |
| 2 | Pendant Silicon-Hydride Functional polydimethyl | H2 | 0.95 |
| | siloxane, trimethylsiloxy terminated, 100 cSt (Andisil XL- | | |
| | 1B, AB Specialty Silicones) | | |
| 3 | Vinyl Terminated Dimethylpolysiloxane, 1000 cSt (Andisil | V1 | 0.11 |
| | VS1000, AB Specialty Silicones) | | |
| 4 | Vinyl Terminated Dimethylpolysiloxane, 5000 cSt (Andisil | V2 | 0.065 |
| | VS5000, AB Specialty Silicones) | | |
| 5 | HMDS treated fumed silica (Aerosil R8200, Evonik) | — | — |
| 6 | HMDS treated fumed silica (Aerosil R812S, Evonik) | — | — |
| 7 | Decamethyl cyclopentasiloxane (Xiameter PMX-0245, | — | — |
| | Dow) | | |
| 8 | Silica (Goddball G-6C, Iwase Cosfa) | — | — |
| 9 | Disiloxane (Xiameter PMX-200, 0.65 cSt, Dow) | — | — |
| 10 | Trisiloxane (and) Dimethicone (Xiameter PMX-1184, | — | — |
| | Dow) | | |

TABLE 12b
F3-96-1 and volatile component variants

| | F3-96-1 | F3-111-1 | F3-112-1 | F3-113-1 |
|---|---|---|---|---|
| No. | Mass (g) | Mass (g) | Mass (g) | Mass (g) |
| 1 | 1.9 | 3.7 | 3.7 | 4.4 |
| 2 | 0.98 | 1.9 | 1.9 | 2.3 |
| 3 | 11.37 | 22.0 | 22.0 | 26.4 |
| 4 | 2.3 | 4.4 | 4.4 | 5.3 |
| 5 | 1.72 | 3.3 | 3.3 | 4.0 |
| 6 | 1.7 | 3.3 | 3.3 | 4.1 |
| 7 | 19.8 | 53.6 | 0.0 | 0.0 |
| 8 | 4 | 7.7 | 7.7 | 9.3 |
| 9 | 7.94 | 0.0 | 0.0 | 64.4 |
| 10 | 0 | 0.0 | 53.6 | 0.0 |
| [H]/[V] | 6.6 | 6.6 | 6.6 | 6.6 |

TABLE 12c
F3-96-1 and volatile component variants

| | F3-96-1 | F3-111-1 | F3-112-1 | F3-113-1 |
|---|---|---|---|---|
| No. | w/w (%) | w/w (%) | w/w (%) | w/w (%) |
| 1 | 3.7% | 3.7% | 3.7% | 3.7% |
| 2 | 1.9% | 1.9% | 1.9% | 1.9% |
| 3 | 22.0% | 22.0% | 22.0% | 22.0% |
| 4 | 4.4% | 4.4% | 4.4% | 4.4% |
| 5 | 3.3% | 3.3% | 3.3% | 3.3% |
| 6 | 3.3% | 3.3% | 3.3% | 3.4% |
| 7 | 38.3% | 53.6% | 0.0% | 0.0% |
| 8 | 7.7% | 7.7% | 7.7% | 7.7% |
| 9 | 15.4% | 0.0% | 0.0% | 53.6% |
| 10 | 0.0% | 0.0% | 53.6% | 0.0% |

TABLE 13

| | | F3-115- | F3-115- | F3-115- | F3-115- | F3-115- | F3-115- |
|---|---|---|---|---|---|---|---|
| | | 1 | 2 | 3 | 1 | 2 | 3 |
| No. | Chemical | Mass (g) | Mass (g) | Mass (g) | w/w (%) | w/w (%) | w/w (%) |
| 1 | MRC (F3-111-1, | 20 | 0 | 0 | 97% | 0% | 0% |
| | STA) | | | | | | |
| 2 | MRC (F3-112- | 0 | 20 | 0 | 0% | 97% | 0% |
| | 1, STA) | | | | | | |
| 3 | MRC (F3-113-1, | 0 | 0 | 20 | 0% | 0% | 97% |
| | STA) | | | | | | |
| 4 | HMDS treated | 0.6 | 0.6 | 0.6 | 3% | 3% | 3% |
| | fumed silica | | | | | | |
| | (Aerosil R812S) | | | | | | |

TABLE 14
Use test

| Site no | Arm | Location | AB | Dosage | MRC | Dosage |
|---|---|---|---|---|---|---|
| 1 | Left | W | F3-102-2 | 1 pump | F3-115-1 | 1 pump |
| 2 | Left | C | F3-102-2 | 1 pump | F3-115-2 | 1 pump |
| 3 | Left | E | F3-102-2 | 1 pump | F3-115-3 | 1 pump |
| 4 | Right | W | F3-102-2 | 1 pump | F3-115-3 | 1 pump |
| 5 | Right | C | F3-102-2 | 1 pump | F3-115-2 | 1 pump |
| 6 | Right | E | F3-102-2 | 1 pump | F3-115-1 | 1 pump |

### Example 9: MRC Stability

The formulations in Tables 12a-12c and Table 13 were placed on stability in a 40° C. incubator. The samples were inspected visually for phase separation and fluidity at each timepoint. At the three month timepoint, only F3-115-1 remained visually uniform as shown in Table 15. Tables 16a-16b demonstrate the cure stability following 2 months.

The AB and MRC were applied to a 3×3 square inch parafilm substrate to generate the adhesive dressing. Visualizable curing on each substrate within 5 minutes confirm the activity of the AB/MRC combination. The measured thickness for each of the cured dressings was in the range of 50 microns to 100 microns based on the caliper readings. The dressings remain adherent to the substrate when subjected to strain, and showed recoil when the substrate was released. Strains of at least 200% were achieved following manual stretching.

TABLE 15
Visual stability at 40° C.

| Formulation | Days | Appearance |
|---|---|---|
| F3-113-1 | 7 | two phases |
| F3-113-1 | 19 | two phases |
| F3-112-1 | 43 | two phases |
| F3-115-2 | 41 | two phases |
| F3-111-1 | 61 | two phases |
| F3-115-1 | 57 | uniform |

TABLE 16a
In vitro characterization of sample stability (2 month)

| | | dose | | | | rapid | |
|---|---|---|---|---|---|---|---|
| Sample | AB | (g) | MRC | dose (g) | AB/ MRC | cure | μm |
| A | F3-102-2 | 0.12 | F3-111-1 | 0.1 | 1.2 | yes | 50-100 |
| B | F3-102-2 | 0.11 | F3-112-1 | 0.12 | 0.9 | yes | 50-100 |
| C | F3-102-2 | 0.09 | F3-115-1 | 0.07 | 1.3 | yes | 50-100 |
| D | F3-102-2 | 0.08 | F3-115-2 | 0.09 | 0.9 | yes | 50-100 |

TABLE 16b
In vitro characterization of sample stability (2 month)

| Sample | Recoil | Elongation | Rub | Toughness Ranking |
|---|---|---|---|---|
| A | yes | >200% | moderate | 2 |
| B | yes | >200% | moderate | 1 |
| C | yes | >200% | moderate | 4 |
| D | yes | >200% | moderate | 3 |

### Example 10: MRC Variants

For the compositions in Table 17a, components (1), (2), (4), (5), and (7) were mixed using a THINKY AR-100 for 30 seconds. Thereafter, Component (3) was introduced. Using the IKA overhead mixer at 800 RPM, component (6) was dispersed into the formulation. The final composition was mixed for 20 minutes at 1000 RPM. F4-1-2 was produced by mixing component (7) into F4-1-2.

TABLE 17a
MRC compositions

| No. | Chemical | Ref. | mmol/g |
|---|---|---|---|
| 1 | Pendant Silicon-Hydride Functional polydimethyl | H1 | 4.35 |
| | siloxane, trimethylsiloxy terminated (Andisil XL-11, AB | | |
| | Specialty Silicones) | | |
| 2 | Vinyl Terminated Dimethylpolysiloxane, 1000 cSt (Andisil | V1 | 0.11 |
| | VS1000, AB Specialty Silicones) | | |
| 3 | HMDS treated fumed silica (Aerosil R8200, Evonik) | — | — |
| 4 | HMDS treated fumed silica (Aerosil R812S, Evonik) | — | — |
| 5 | Decamethyl cyclopentasiloxane (Xiameter PMX-0245, | — | — |
| | Dow) | | |
| 6 | Silica (Goddball G-6C, Iwase Cosfa) | — | — |
| 7 | Polydimethylsiloxane fluid (PMX-200, 5 cSt, Dow) | — | — |

TABLE 17b
MRC compositions

| | F4-1-1 | F4-1-2 | F4-4-1 | F4-1-1 | F4-1-2 | F4-4-1 |
|---|---|---|---|---|---|---|
| No. | Mass (g) | Mass (g) | Mass (g) | w/w (%) | w/w (%) | w/w (%) |
| 1 | 2.7 | 2.6 | 1.0 | 5.4% | 5.1% | 5.1% |
| 2 | 15.9 | 15.2 | 6.1 | 32.5% | 30.4% | 30.2% |
| 3 | 1.9 | 1.9 | 0.7 | 3.9% | 3.7% | 3.7% |
| 4 | 1.9 | 4.3 | 1.7 | 3.8% | 8.6% | 8.5% |
| 5 | 21.4 | 21.4 | 8.5 | 43.6% | 42.7% | 42.4% |
| 6 | 5.0 | 4.7 | 1.9 | 10.1% | 9.4% | 9.4% |
| 7 | 0.29 | 0.00 | 0.14 | 0.6% | 0.0% | 0.7% |
| [H]/[V] | 6.6 | 6.7 | 6.7 | — | — | — |

### Example 11: Management of Toddler Eczema

The AB F2-48-2 and the MRC F2-37-1 were applied to eczematous skin at the knee flexural sites of a 2 year old. A thin layer of the AB was applied to the target skin site using a silicone applicator. A thin coating of the MRC was then applied over the AB using a metal spatula. The formulations were applied twice daily, after bathing or cleansing the treated skin areas with a wet towel and then dabbing the skin dry.

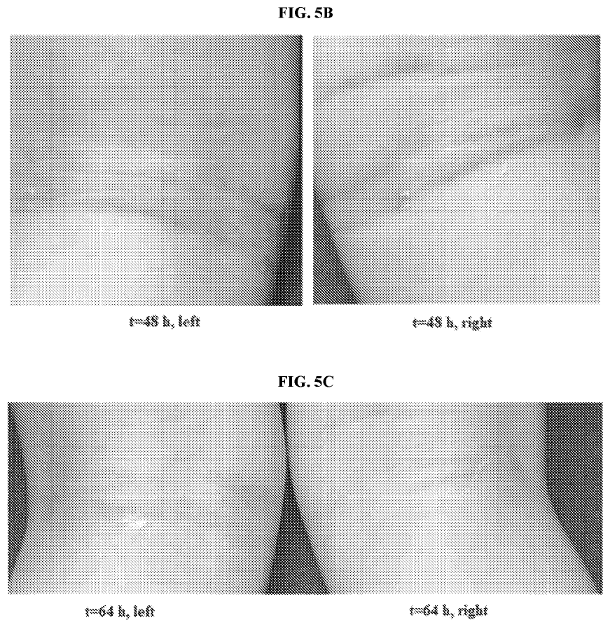

FIGS. 5A-5C shows the improvement in the right and the left skin sites at 48 hours and 64 hours after the initial application of the adhesive dressing at t=0. The improvement in the skin sites were also confirmed by dermatologist's live examination of the application areas. Note that adherent remnants of the dressing may be seen in the photographs, as the skin sites were not cleaned prior to taking the photographs.

The final compositions of F2-47-1 and F2-48-2 are shown in Tables 18a-18b. F2-47-1 was prepared as follows. Components 1, 2, 3, 4 and 5 were mixed to 10 seconds and allowed to react for 15 minutes. Component 6 was then introduced and mixed for 20 seconds. Twenty-five minutes after the initiation of the crosslinking reaction, Component 8 was added and mixed for 20 seconds. Component 7 was then introduced and mixed for 20 seconds. After 46 minutes from the start of the reaction, the viscosity of F2-47-1 was measured over 12 minutes using an SP-4 spindle at 2.5 RPM (IKA lo-vi visc) and ranged from 68872 cP to 74631 cP.

F2-48-2 was prepared by mixing 6.22 g of F2-47-1 with 3.13 g of Component 9.

F2-37-1 (Table 19) was prepared by mixing Components 1, 2, 3 and 4.

TABLE 18a
F2-48-2 AB composition

| No. | Chemical | Ref. | mmol/g |
|---|---|---|---|
| 1 | 0.8-1.2% vinylmethylsiloxane dimethylsiloxane | V1 | 0.13 |
| | copolymer, trimethylsiloxy terminated, 800-1200 cSt | | |
| | (VDT-131, Gelest) | | |
| 2 | hydride terminated polydimethylsiloxane, 10,000 cSt | H1 | 0.031 |
| | (DMSH-41, Gelest) | | |
| 3 | dimethylpolysiloxane, 50 cSt (DM50, Innospec) | — | — |
| 4 | C13-15 Alkane (and) Isododecane (and) Caprylyl | — | — |
| | Methicone (CHT-BeauSil Fluid 8301, CHT USA) | | |
| 5 | 2000 ppm Karstedt's catalyst in KF995 (F1-86-1, STA) | — | — |
| 6 | vinyl terminated dimethylpolysiloxane, 20,000 cSt | V2 | 0.04 |
| | (VS20000, Andisil) | | |
| 7 | 2000 ppm Karstedt's catalyst in KF995 (F1-86-1, STA) | — | — |
| 8 | vinyl terminated dimethylpolysiloxane, 165,000 cSt | V3 | 0.015 |
| | (VS165000, Andisil) | | |
| 9 | Decamethylcyclopentasiloxane (KF995, Shin Etsu) | — | — |

TABLE 18b
F2-48-2 AB composition

| | F2-47-1 | F2-48-2 | F2-47-1 | F2-48-2 |
|---|---|---|---|---|
| No. | Mass (g) | Mass (g) | w/w (%) | w/w (%) |
| 1 | 1.26 | 0.39 | 6.3% | 4.2% |
| 2 | 5.29 | 1.63 | 26.3% | 17.5% |
| 3 | 0.70 | 0.22 | 3.5% | 2.3% |
| 4 | 1.32 | 0.41 | 6.6% | 4.4% |
| 5 | 0.43 | 0.13 | 2.1% | 1.4% |
| 6 | 7.41 | 2.29 | 36.8% | 24.5% |
| 7 | 0.50 | 0.15 | 2.5% | 1.7% |
| 8 | 3.22 | 0.99 | 16.0% | 10.6% |
| 9 | 0.00 | 3.13 | 0.0% | 33.5% |
| [H]/[V] | 0.32 | — | — | — |

TABLE 19
F2-37-1 MRC composition

| No. | Chemical | Ref. | mmol/g | Mass (g) | w/w (%) |
|---|---|---|---|---|---|
| 1 | Pendant Silicon-Hydride | H1 | 4.35 | 0.45 | 2% |
| | Functional polydimethyl | | | | |
| | siloxane, trimethylsiloxy | | | | |
| | terminated (Andisil XL-11, | | | | |
| | AB Specialty Silicones) | | | | |
| 2 | Pendant Silicon-Hydride | H2 | 0.95 | 0.72 | 4% |
| | Functional polydimethyl | | | | |
| | siloxane, trimethylsiloxy | | | | |
| | terminated, 100 cSt (Andisil | | | | |
| | XL-1B, AB Specialty | | | | |
| | Silicones) | | | | |
| 3 | Vinyl Terminated | V1 | 0.11 | 3.45 | 17% |
| | Dimethylpolysiloxane, 1000 | | | | |
| | cSt (Andisil XL-1B, AB | | | | |
| | Specialty Silicones) | | | | |
| 4 | Disiloxane (Belsil DM 0.65 | — | — | 15.38 | 77% |
| | cSt, Wacker | | | | |

### Example 12: F3-102-1 and F3-96-1 Application to Skin

### Protection of Facial Abrasion

F3-102-1 (AB) and F3-96-1 (MRC) were applied to a facial abrasion, as follows. One 0.12 mL pump of the AB was applied to a metal roller and rolled over the target skin area to form a thin uniform coating. Two drops of the MRC were applied to a metal applicator using an eye dropper package. Using the metal applicator, the MRC was applied over the AB, starting at the center of the skin application site and then sweeping the MRC toward the AB periphery, to uniformly coat the AB. Following 5 minutes of MRC application, the ASD demonstrated minimal tack.

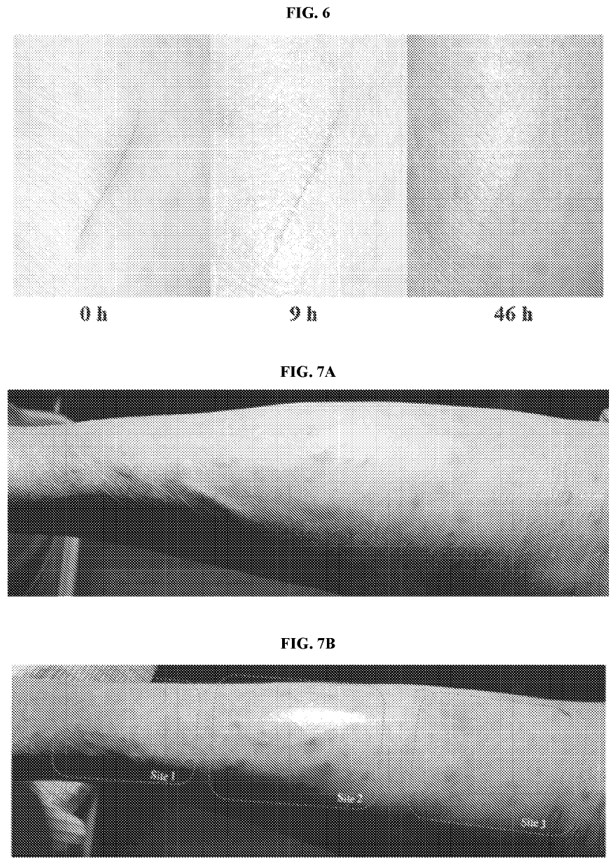

The ASD was applied after bathing and removed after 20 hours of wear prior to reapplication after bathing again on the second day of wear. FIG. 6 shows the facial abrasion at baseline (left panel), at 9 hours of ASD wear (center panel), and the abrasion site 46 hours after ASD wear (right panel). A visible improvement in the appearance of the facial scratch was observed following 2 days of ASD wear.

### Protection of Eczematous Skin

Following the methods described, the AB/MRC combination was applied to eczema lesions on the left wrist and the left hand of a 3-year old. Following overnight wear (approximately 16 hours), the 50% of the ASD applied to the wrist persisted, whereas the ASD applied to the top of the hand had was worn off. Upon review by a board certified dermatologist, the inflammation present at the application sites had resolved. The improved skin appearance was possibly due to ASD induced reduction epidermal water loss and intervention in the itch-scratch cycle by forming a protective layer on the skin.

The ASD was reapplied to the same hand and wrist sites of the 3-year old after bathing and worn overnight. After 20 hours of wear, the ASD remained present on the wrist. The ASD worn on the hand was no longer present after 12 hours of wear. Inflammation was not present at either site.

### Example 13: Dry Leg User Study

A 79 year old female volunteer who was self-diagnosed with dry legs participated in the study. The formulation application scheme is provided in Table 20.

The skin application sites were gently wiped with a warm towel and then dabbed dry with a tissue. The four corners of a 9 square inch area were marked on the skin for each designated skin test site The AB was dispensed onto a metal roller and then applied to the designated skin site by rolling the applicator with gentle pressure to transfer the formulation onto the skin. The MRC (F3-115-1) was then dispensed onto a metal applicator spatula, which was then used to coat the MRC onto the AB on the skin. The mass of each applicator before and after application was measured to calculate the mass of each respective formulation applied. These values also are reported in Table 20.

After the photographs were taken, the volunteer was allowed to freely engage in her daily routine, including bathing and sleeping.

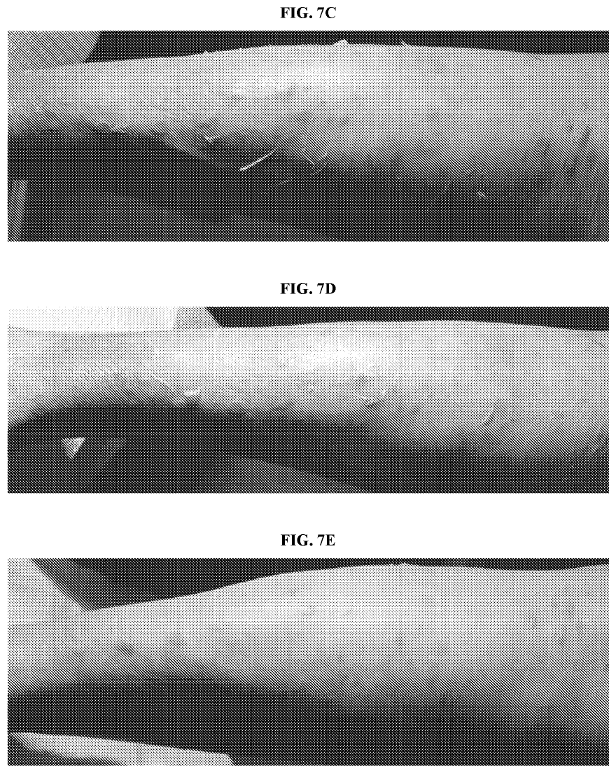

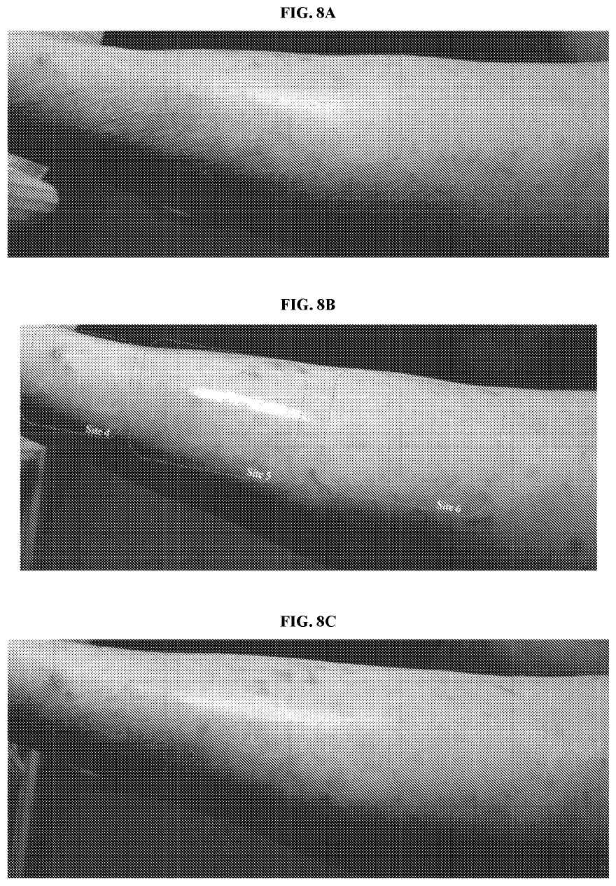

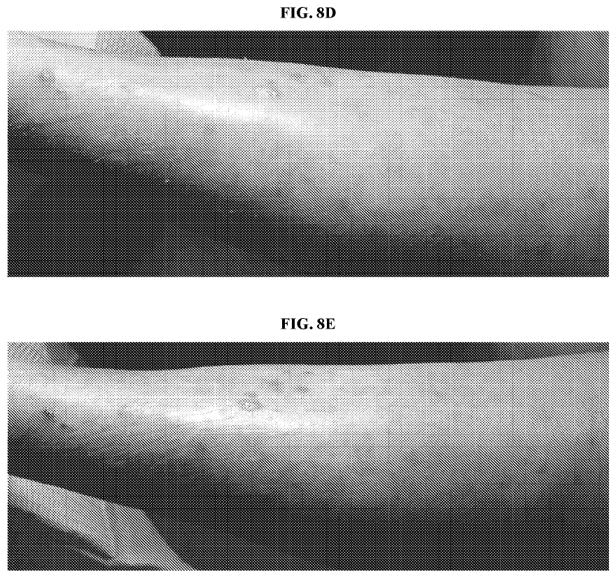

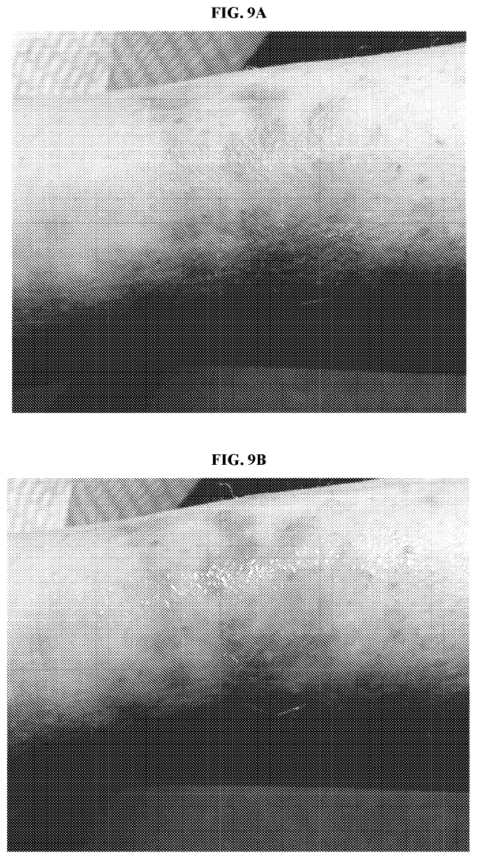

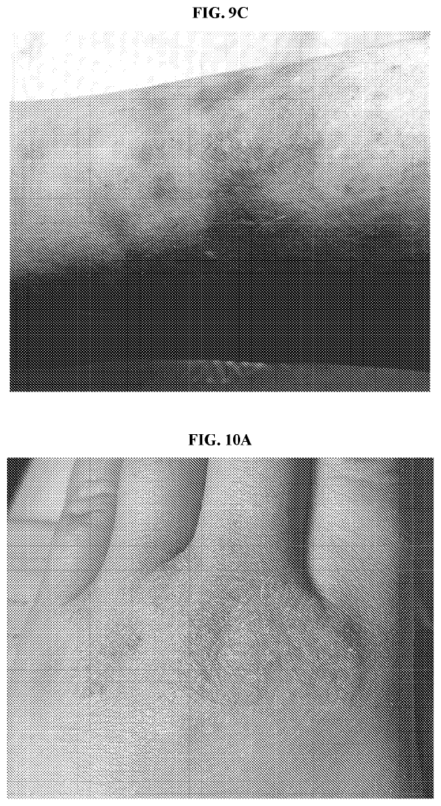

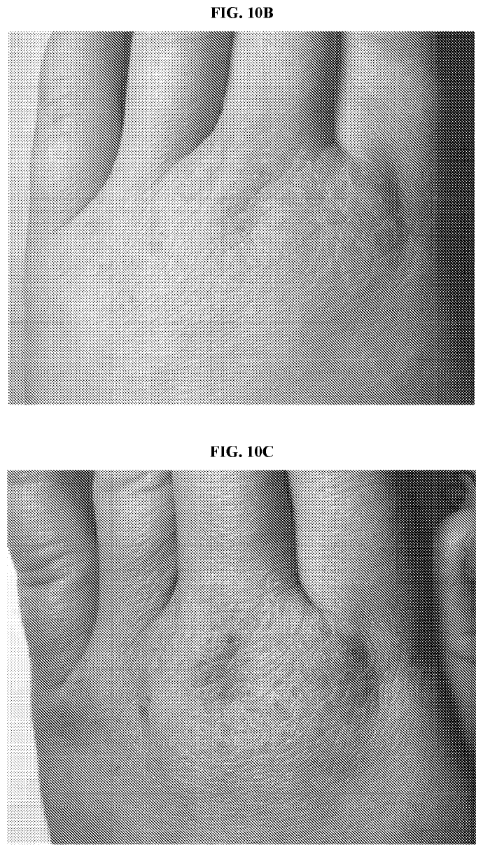

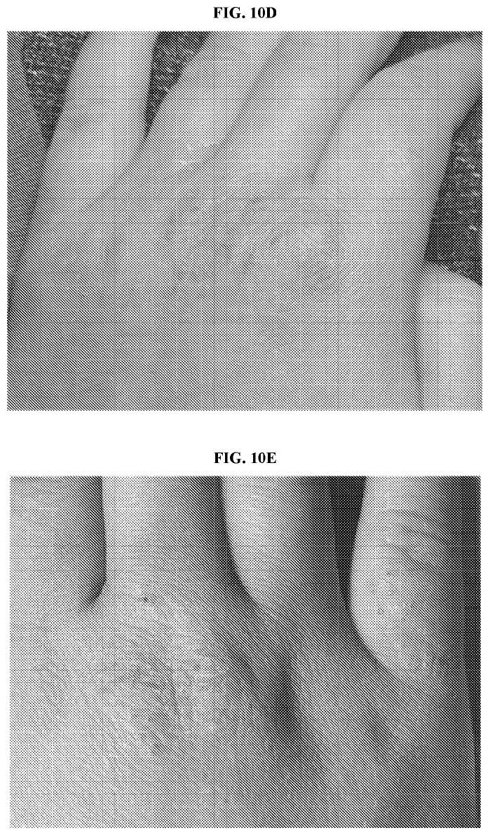

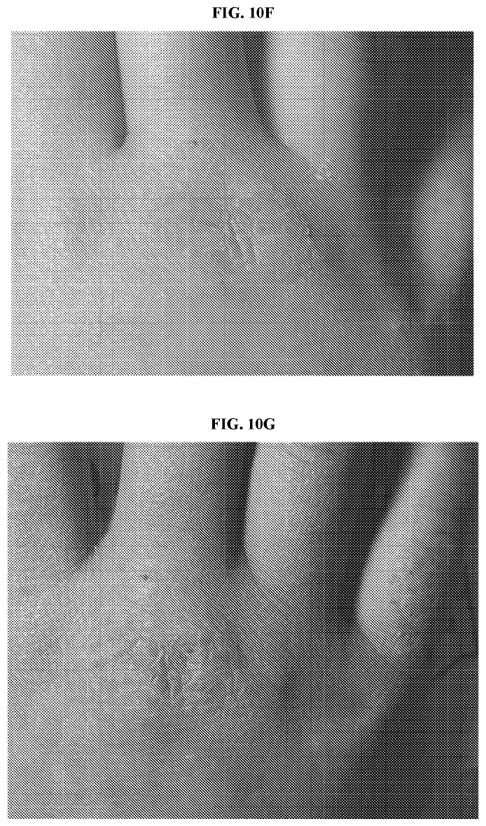

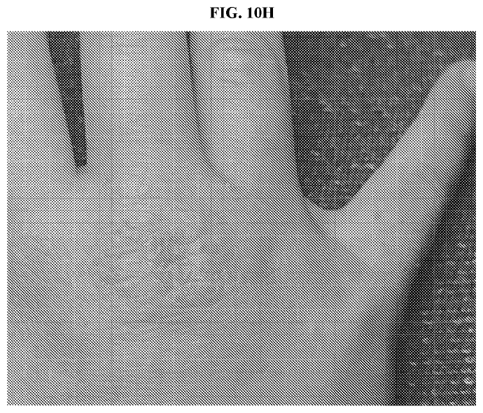

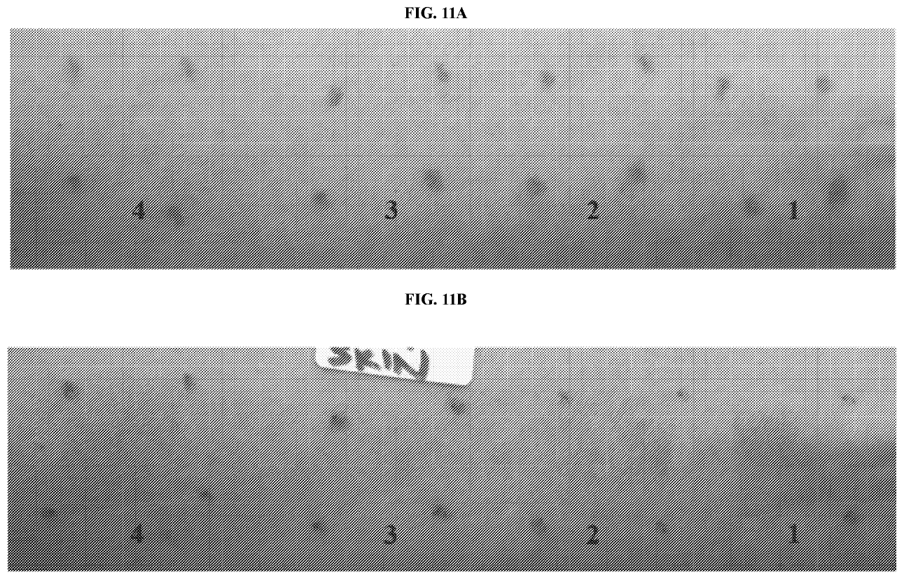

Photographs were taken of the skin prior to formulation application (baseline) and then after 2 minutes, one day and two days of wearing the adhesive dressing. A final photograph was taken at 48 hours following test article application to evaluate the duration of the improved skin appearance following removal of the dressings. The test sites are demarcated with dashed lines in the photographs corresponding to 2 minutes post application in FIGS. 7A-7E and FIGS. 8A-8E for the right and the left legs, respectively.

The formulations on the volunteer's left leg did not persist through day one. Based on a visual and tactile examination of the application site by the volunteer, the skin surface did feel and appear notably smoother compared to the baseline condition at each site.

The three formulations on the volunteer's right leg remained adherent over 24 hours, with greater than 90% of the dressing present. After 48 hours of wear, approximately 60% of the dressing remained adherent to the skin. On the right leg, site 2 showed the greatest extent of film lifting. Site 3 showed mild lifting of the cured dressing and the best durability of the three sites. The dressing was removed from the right leg after taking photographs at the 48 hour time point and a final photograph of the right leg was taken two days following test article removal.

For both the left and the right legs, the photographs taken at 4 days after test article application showed a sustained improvement in the treated skin appearance compared to the baseline photographs with respect to the surface texture.

On average, the AB quantity applied and the AB/MRC values were 2.2 mg/cm² and 86%, respectively, for the right leg application sites. For the left leg, the corresponding values were substantially lower, at 1.7 mg/cm² and 68%.

TABLE 20
Formulation Application scheme and AB/MRC mass ratios

| Leg location | Site No. | AB | AB (g) | F3-115-1 MRC (g) | AB/MRC (w/w) |
|---|---|---|---|---|---|
| RL | 1 | F3-137-1 | 0.12 | 0.14 | 86% |
| RC | 2 | F3-134-1 | 0.12 | 0.15 | 80% |
| RU | 3 | F3-136-1 | 0.14 | 0.15 | 93% |
| LL | 4 | F3-134-1 | 0.11 | 0.14 | 79% |
| LC | 5 | F3-136-1 | 0.1 | 0.15 | 67% |
| LU | 6 | F3-137-1 | 0.09 | 0.15 | 60% |

### Example 14: Radiation Dermatitis Application

The AB and MRC pair of F3-136-1 and F3-115-1 were applied sequentially, using a metal roller followed by a metal spatula, to a 9 square inch skin area on the right leg of an 85 year old volunteer experiencing radiation dermatitis. The AB/MRC value was 86%, where the AB coverage was 2.1 mg/cm². Photographs were taken at baseline, 2 minutes post application and 2 days post application. The volunteer was allowed to engage in his daily routine 5 minutes after applying the dressing.

The volunteer self-assessment noted an improvement in the skin dryness following use and a reduction of itch following one day of use. The photograph at 2 days after application showed that the areas of dry skin, as seen by the white reflective regions in the baseline photographs, were completely treated with the adhesive dressing. This observation is consistent with previous reports wherein hydrated skin showed an increase in skin translucency and a decrease in the skin surface scattering.

### Example 15: Eczematous Skin Application

F3-136-1 and F3-115-1 were applied to eczematous lesions present on the dorsal hand region of a 3 year old using a metal roller to apply the AB and a metal spatula to apply the MRC. One full pump of each formulation was used for both hand application sites. The AB and MRC formulations were applied as needed over the course of four days. Photography was taken at baseline and each morning after overnight wear of the dressing.

### Example 16: Preparation of Exemplary Adhesive Base Compositions

### AB2-004

AB2-004 (Table 21) was prepared as follows. Briefly, RM 1, RM 2, and RM 5 were mixed for 10 minutes at 300 rpm with an overhead mixer. RM 6 was added to the main vessel and mixed for 1 hour at 300 rpm. RM 3 and RM 7 were mixed with a spatula until a clear mixture was visible and then mixed with the contents of the main vessel for 2 hours at 300 rpm. Following 23 hours, RM 4 was added to the main vessel and mixed for 1 hour and 40 minutes at 300 rpm. 43 hours later, the viscosity was measured using the methods described elsewhere herein, indicating a viscosity of 4979 cP.

TABLE 21
AB2-004 adhesive base composition

| No. | Chemical | Ref. | mmol/g | Mass (g) | w/w (%) |
|---|---|---|---|---|---|
| 1 | 0.8-1.2% vinylmethylsiloxane | V1 | 0.13 | 35.8 | 8.9% |
| | dimethylsiloxane copolymer, | | | | |
| | trimethylsiloxy terminated, 800-1200 cSt | | | | |
| | (VDT-131, Gelest) | | | | |
| 2 | monohydride terminated | H1 | 0.1 | 23.3 | 5.8% |
| | polydimethylsiloxane, asymmetric, 150- | | | | |
| | 250 cSt (MCR-H22, Gelest) | | | | |
| 3 | hydride terminated polydimethylsiloxane | H2 | 0.031 | 37.6 | 9.4% |
| | 10,000 cSt (DMS H-41, Gelest) | | | | |
| 4 | vinyl terminated dimethylpolysiloxane, | V3 | 0.024 | 96.7 | 24.2 |
| | 80,000 cSt (VS 80000, AB Specialty | | | | |
| | Silicones) | | | | |
| 5 | polydimethylsiloxane 95-105 mPa · s | | | 75.6 | 18.9% |
| | (Belsil DM100, Wacker) | | | | |
| 6 | 3000 ppm Karstedt's Catalyst (SIP6830.3, | | | 9.7 | 2.4% |
| | Gelest) | | | | |
| 7 | decamethylcyclopentasiloxane (Andisil | | | 121.4 | 30.3% |
| | D5, AB Specialty Silicones) | | | | |

### AB4-002

AB4-002 (Table 22) was prepared by mixing RM 3 and RM 1 for 10 minutes at 300 rpm using an overhead mixer. RM 2 was then introduced dropwise at 400 rpm. The resulting mixture was mixed for 1 hour at 300 rpm.

TABLE 22
AB4-002 adhesive base composition

| No. | Chemical | Mass (g) | w/w (%) |
|---|---|---|---|
| 1 | AB2-004 | 200.0 | 96.6% |
| 2 | Glycerin (Acme Hardesty) | 3.5 | 1.7% |
| 3 | Cetyl diglyceryl tris(trimethylsiloxy)silylethyl | 3.5 | 1.7% |
| | dimethicone (Dowsil ES5600, Dow Chemical | | |
| | Company) | | |

### AB5-001

AB5-001 (Table 23) was prepared by dispersing aliquots of RM 2 into RM 1 using a planetary centrifugal mixer (Hauschild Speedmixer. Hamm, Germany) using “program 1” (i.e., 1000 rpm for 30 seconds, 1750 rpm for 60 seconds, and 800 rpm for 10 seconds) until a uniform dispersion was visualized.

TABLE 23
AB5-001 adhesive base composition

| No. | Chemical | Mass (g) | w/w (%) |
|---|---|---|---|
| 1 | AB2-004 | 301.6 | 90.9% |
| 2 | HMDS treated fumed silica (Cabosil TS530, Cabot | 30.16 | 9.1% |
| | Corporation | | |

### AB6-001

To prepare AB6-001 (Table 24), RM 1 and RM 6 were mixed (IKA Eurostar 60, 400 RPM, 5 min). RM 2 was added (2 hours mixing at 100 RPM). RM 5 was mixed into the batch (10 min at 300 RPM). RM 3 was then slowly introduced (400 RPM, 5 min). The Step 2 product was mixed 5 times at 30 minute intervals (Hauschild Speedmixer) with “program 1” (1000 rpm for 30 seconds, 1750 rpm for 60 seconds, and 800 rpm for 10 seconds). RM 4 was incorporated using program 1 twice over 30 mins. The final product was mixed again after 18 hours.

TABLE 24
AB6-001 adhesive base composition

| | | | | Mass | |
|---|---|---|---|---|---|
| No. | Chemical | Ref. | mmol/g | (g) | w/w (%) |
| 1 | 0.8-1.2% vinylmethylsiloxane | V1 | 0.11 | 25 | 15.5% |
| | dimethylsiloxane copolymer, | | | | |
| | trimethylsiloxy terminated, 800-1200 cSt | | | | |
| | (VDT-131, Gelest) | | | | |
| 2 | monohydride terminated | H1 | 0.1 | 13.8 | 8.5% |
| | polydimethylsiloxane, asymmetric, 150- | | | | |
| | 250 cSt (MCR-H22, Gelest) | | | | |
| 3 | hydride terminated polydimethylsiloxane | H2 | 1.8 | 0.43 | 0.3% |
| | 7-10 cSt (DMS H-11, Gelest) | | | | |
| 4 | vinyl terminated dimethylpolysiloxane, | V3 | 0.03 | 57.3 | 35.5% |
| | 80,000 cSt (Andisil VS 80000, AB | | | | |
| | Specialty Silicones) | | | | |
| 5 | decamethylcyclopentasiloxane (Andisil | | | 60.2 | 37.3% |
| | D5, AB Specialty Silicones) | | | | |
| 6 | 3000 ppm Karstedt's Catalyst (SIP6830.3, | | | 4.8 | 3.0% |
| | Gelest) | | | | |

### AB F5-41-1A

Adhesive base composition AB F5-41-1A (Tables 25a-25b) was prepared following the methods described for AB6-001. The process step viscosities for AB6-001 and AB F5-41-1A are provided in Table 85b.

TABLE 25a
AB F5-41-1A adhesive base composition

| No. | Chemical | Ref. | mmol/g | Mass (g) | w/w (%) |
|---|---|---|---|---|---|
| 1 | 0.8-1.2% vinylmethylsiloxane | V1 | 0.11 | 93.4 | 18.90% |
| | dimethylsiloxane copolymer, | | | | |
| | trimethylsiloxy terminated, 800-1200 cSt | | | | |
| | (VDT-131, Gelest) | | | | |
| 2 | monohydride terminated | H1 | 0.1 | 51.4 | 10.40% |
| | polydimethylsiloxane, asymmetric, 150- | | | | |
| | 250 cSt (MCR-H22, Gelest) | | | | |
| 3 | hydride terminated polydimethylsiloxane | H2 | 1.8 | 1.4 | 0.30% |
| | 7-10 cSt (DMS H-11, Gelest) | | | | |
| 4 | vinyl terminated dimethylpolysiloxane, | V3 | 0.03 | 107 | 21.60% |
| | 80,000 cSt (Andisil VS 80000, AB | | | | |
| | Specialty Silicones) | | | | |
| 5 | decamethylcyclopentasiloxane (Andisil | | | 223.1 | 45.10% |
| | D5, AB Specialty Silicones) | | | | |
| 6 | 3% Pt Karstedt's Catalyst (SIP6830.3, | | | 18.03 | 3.60% |
| | Gelest) | | | | |

TABLE 25b
Summary of reaction step viscosities

| | Process | viscosity | Spindle | | Time |
|---|---|---|---|---|---|
| Formulation | Step | (cP) | No. | RPM | (min) |
| AB6-001 | 1 | 1334 | 3 | 50 | 2 |
| AB6-001 | 2 | 200 | 3 | 100 | 2 |
| AB6-002 | 3 | 3864 | 3 | 10 | 2 |
| F5-41-1A | 1 | 1334 | 3 | 50 | 2 |
| F5-41-1A | 2 | 200 | 3 | 100 | 2 |
| F5-41-1A | 3 | 1392 | 3 | 10 | 2 |

TABLE 26
Selected vinyl and hydride component ratios
for certain adhesive base compositions

| Ratio | AB2-002 | AB2-004 | AB6-001 | F5-41-1A |
|---|---|---|---|---|
| H/V | 0.5 | 0.5 | 0.5 | 0.6 |
| H1/V1 | 0.5 | 0.5 | 0.5 | 0.5 |
| H2/H1 | 0.5 | 0.5 | 0.6 | 0.5 |
| V3/H2 | 2.2 | 2 | 2.2 | 1.3 |

### Exemplary AB6-001 Variants

Variations of adhesive base composition AB6-001 (Table 27) were prepared to assess the effects of additives on modifying the performance attributes of adhesive dressings. For F5-16-01, the fumed silica (RM2) was incorporated into AB6-001 (RM1) using a THINKY AR-100 mixer for 20 seconds. Subsequent lots of F5-16-01 (i.e., AB7-001) were produced at a scale of >250 g, wherein program 1 of the planetary centrifugal mixer (Hauschild Speedmixer) was used to incorporate the fumed silica. R 3 was dispersed into the formulation at 1000 rpm for 30 seconds using the same mixer.

TABLE 27
AB6-001 variant adhesive base compositions

| | | F5-16-01 | | F5-37-1 | | F5-64-1 | |
|---|---|---|---|---|---|---|---|
| | | Mass | w/w | Mass | w/w | Mass | w/w |
| No. | Chemical Name | (g) | (%) | (g) | (%) | (g) | (%) |
| 1 | AB6-001 | 10 | 91% | 341 | 95% | 27.6 | 89% |
| 2 | HMDS treated fumed silica (Cabosil | 1 | 9% | 17 | 5% | 2.8 | 9% |
| | TS530, Cabot Corporation) | | | | | | |
| 3 | Dimethicone (Andisil DM 100, AB | 0 | 0 | 0 | 0 | 0.6 | 2% |
| | Specialty Silicones) | | | | | | |

### AB8-001

A 600 g batch of AB8-001 (F5-54-1 A) was prepared according to the methods described elsewhere herein (Table 28).

TABLE 28
AB8-001 adhesive base composition

| No. | Chemical | Ref. | mmol/g | Mass (g) | w/w (%) |
|---|---|---|---|---|---|
| 1 | 0.8-1.2% vinylmethylsiloxane | V1 | 0.11 | 58.4 | 17.18 |
| | dimethylsiloxane copolymer, | | | | |
| | trimethylsiloxy terminated, 800-1200 cSt | | | | |
| | (VDT-131, Gelest) | | | | |
| 2 | monohydride terminated | H1 | 0.1 | 32.1 | 9.45 |
| | polydimethylsiloxane, asymmetric, 150- | | | | |
| | 250 cSt (MCR-H22, Gelest) | | | | |
| 3 | hydride terminated polydimethylsiloxane | H2 | 1.8 | 0.9 | 0.26 |
| | 7-10 cSt (DMS H-11, Gelest) | | | | |
| 4 | vinyl terminated dimethylpolysiloxane, | V3 | 0.03 | 66.9 | 19.69 |
| | 80,000 cSt (Andisil VS 80000, AB | | | | |
| | Specialty Silicones) | | | | |
| 5 | decamethylcyclopentasiloxane (Andisil | | | 149.6 | 44.00 |
| | D5, AB Specialty Silicones) | | | | |
| 6 | 3% Pt Karstedt's Catalyst (SIP6830.3, | | | 1.1 | 0.33 |
| | Gelest) | | | | |

### Exemplary AB8-001 Variants

Variations of adhesive base composition AB8-001 (Table 29) (i.e., a 600 g batch of F5-54-1A) were prepared by processing AB8-001 according to Table 29.

TABLE 29
AB8-001 variant adhesive base compositions

| | | F5-78-1 | | F5-78-2 | | F5-79-1 | |
|---|---|---|---|---|---|---|---|
| | | Mass | w/w | Mass | w/w | Mass | w/w |
| No. | Chemical Name | (g) | (%) | (g) | (%) | (g) | (%) |
| 1 | AB8-001 | 250 | 95.2 | 309 | 90.9 | 72 | 90.0 |
| 2 | HMDS treated fumed silica (Cabosil | 12.5 | 4.8 | 30.9 | 9.1 | 7.2 | 9.0 |
| | TS530, Cabot Corporation) | | | | | | |
| 3 | Dimethicone (Andisil SF 100, AB | 0 | 0.0 | 0 | 0.0 | 0.8 | 1.0 |
| | Specialty Silicones) | | | | | | |

TABLE 30
AB8-001 formulation viscosities

| | AB8-001 | F5-78-1 | F5-78-2 | F5-79-1 |
|---|---|---|---|---|
| Viscosity (cPs), SP-3 spindle | 1603 | 4895 | 40615 | 39715 |
| RPM | 50 | 5 | 2 | 2 |
| Time (min) | 1 | 1 | 2 | 2 |

### Example 17: Preparation of Exemplary Mechanically Reinforcing Compositions (MRCs) MRC2-003

MRC2-003 (Table 31) was prepared by first mixing RM 1, RM 2, RM 4, and RM 6 (i.e., components 1, 2, 4, and 6). RM 3 was then introduced and mixed using a planetary centrifugal mixer (Hauschild Speedmixer, Hamm, Germany) until a uniform dispersion was observed. RM 5 was next introduced and similarly mixed to afford MRC2-003.

TABLE 31
MRC2-003 mechanically reinforcing composition

| No. | Chemical | Ref. | mmol/g | Mass (g) | w/w (%) |
|---|---|---|---|---|---|
| 1 | Pendant Silicon-Hydride Functional | H1 | 4.1 | 20.8 | 5.05% |
| | polydimethyl siloxane, trimethylsiloxy | | | | |
| | terminated (Andisil XL-11, AB Specialty | | | | |
| | Silicones) | | | | |
| 2 | Vinyl terminated dimethylpolysiloxane, | V1 | 0.121 | 123.4 | 29.98% |
| | 1000 cSt (Andisil VS1000, AB Specialty | | | | |
| | Silicones) | | | | |
| 3 | HMDS treated fumed silica (Cabosil | | | 49.9 | 12.12% |
| | TS530, Cabot Corporation) | | | | |
| 4 | Decamethylcyclopentasiloxane (Xiameter | | | 176.1 | 42.78% |
| | PMX-0245, Dow Chemical Company) | | | | |
| 5 | Silica (Goddball G-6C, Iwase Cosfa) | | | 38.5 | 9.35% |
| 6 | Polydimethylsiloxane fluid, 5 cSt (PMX- | | | 2.9 | 0.70% |
| | 200, Dow Chemical Company) | | | | |

### Exemplary MRC2-003 Variants

Variations of mechanically reinforcing composition MRC2-003 were prepared to assess the effects of vinyl siloxane length and hydride concentration on the mechanical properties of cured layers (Tables 32a-32c).

TABLE 32a
MRC2-003 mechanically reinforcing composition

| | | | mmol/ |
|---|---|---|---|
| No. | Chemical Name | Ref. | g |
| 1 | Pendant Silicon-Hydride Functional polydi- | H1 | 4.1 |
| | methylsiloxane, trimethylsiloxy terminated | | |
| | (Andisil XL-11, AB Specialty Silicones) | | |
| 2 | Hydride Terminated Polydimethylsiloxane, 7-10 | H2 | 1.8 |
| | cSt (DMS H-11, Gelest) | | |
| 3 | Pendant Silicon-Hydride Functional polydi- | H3 | 0.95 |
| | methylsiloxane, trimethylsiloxy terminated | | |
| | (Andisil XL-1B, AB Specialty Silicones) | | |
| 4 | Vinyl terminated dimethylpolysiloxane, 1000 cSt | V1 | 0.121 |
| | (Andisil VS1000, AB Specialty Silicones) | | |
| 5 | Vinyl terminated dimethylpolysiloxane, 80000 cSt | V2 | 0.03 |
| | (Andisil VS80000, AB Specialty Silicones) | | |

TABLE 32b

| | Component No. | | | | |
|---|---|---|---|---|---|
| | 1 | 2 | 3 | 4 | 5 |
| Formulation | Mass (g) | Mass (g) | Mass (g) | Mass (g) | Mass (g) |
| F5-18-00 | 5 | 0 | 0 | 29.7 | 0 |
| F5-18-01 | 4.8 | 0 | 0 | 26 | 10.5 |
| F5-18-02 | 4.6 | 0 | 0 | 26.1 | 5 |
| F5-18-03 | 4 | 0.9 | 0 | 26.2 | 0 |
| F5-18-04 | 4 | 2.7 | 0 | 30.9 | 0 |
| F5-18-05 | 0 | 0 | 10 | 13.8 | 0 |
| F5-20-1 | 2.6 | 0 | 0 | 13 | 10.54 |
| F5-20-2 | 1.7 | 0 | 0 | 5 | 20.23 |
| F5-20-3 | 0 | 0 | 16 | 20 | 8.1 |
| F5-20-4 | 0 | 0 | 7 | 8 | 6.55 |
| F5-20-5 | 0 | 0 | 5.8 | 4 | 16.1 |

TABLE 32c

| | Component No. | | | | |
|---|---|---|---|---|---|
| | 1 | 2 | 3 | 4 | 5 |
| Formulation | w/w (%) | w/w (%) | w/w (%) | w/w (%) | w/w (%) |
| F5-18-00 | 14.4 | 0 | 0 | 85.6 | 0 |
| F5-18-01 | 11.6 | 0 | 0 | 63.0 | 25.4 |
| F5-18-02 | 12.9 | 0 | 0 | 73.1 | 14.0 |
| F5-18-03 | 12.9 | 2.9 | 0 | 84.2 | 0 |
| F5-18-04 | 10.6 | 7.2 | 0 | 82.2 | 0 |
| F5-18-05 | 0 | 0 | 42.0 | 58.0 | 0 |
| F5-20-1 | 9.9 | 0 | 0 | 49.7 | 40.3 |
| F5-20-2 | 6.3 | 0 | 0 | 18.6 | 75.1 |
| F5-20-3 | 0 | 0 | 36.3 | 45.4 | 18.4 |
| F5-20-4 | 0 | 0 | 32.5 | 37.1 | 30.4 |
| F5-20-5 | 0 | 0 | 22.4 | 15.4 | 62.2 |

### Example 18: Hardness of exemplary mechanically reinforcing compositions

Slabs of the MRC2-003 variant compositions described in Example 17 (Tables 32a-32b) were prepared by mixing the MRC composition with an inhibitor solution, followed by introduction of catalyst (3000 ppm Karstedt catalyst; Table 33). The inhibitor solution (F5-10-1) was prepared from 1,3-divinyl tetramethyl disiloxane (Andisil 2827-186L; AB Silicones, Waukegan, IL) and decamethylcyclopentasiloxane (1:39 w/w ratio).

TABLE 33
Exemplary slabs comprising mechanically reinforcing compositions

| | Components and/or Amounts | | | |
|---|---|---|---|---|
| Slab | MRC | MRC | F5-10-1 | Catalyst |
| Formulation | Formulation | Amount (g) | (g) | (g)^{a} |
| S5-18-00 | F5-18-00 | 2.9 | 0.07 | 0.02 |
| S5-18-01 | F5-18-01 | 2.9 | 0.07 | 0.02 |
| S5-18-02 | F5-18-02 | 2.9 | 0.07 | 0.02 |
| S5-18-03 | F5-18-03 | 2.9 | 0.07 | 0.02 |
| S5-18-04 | F5-18-04 | 2.9 | 0.07 | 0.04 |
| S5-18-05 | F5-18-05 | 2.9 | 0.07 | 0.01 |
| S5-20-1 | F5-20-1 | 2.9 | 0.1 | 0.01 |
| S5-20-2 | F5-20-2 | 2.9 | 0.1 | 0.01 |
| S5-20-3A | F5-20-3 | 2.9 | 0.1 | 0.01 |
| S5-20-3B | F5-20-3 | 2.9 | 0 | 0.01 |
| S5-20-4 | F5-20-4 | 2.9 | 0.1 | 0.01 |
| S5-20-5 | F5-20-5 | 2.9 | 0.1 | 0.01 |

^{a}Karstedt Catalyst (3000 ppm).

The resulting composition was then cured overnight. The slab harness values were measured using a Shore 00 Durometer at 5 different locations (AD-100, Checkline, Lynbrook, NY), where the average hardness values are summarized in Table 34.

TABLE 34
Shore 00 hardness of exemplary slab compositions

| Slab | Measurement No. | | | | | | | StDev |
|---|---|---|---|---|---|---|---|---|
| Formulation | 1 | 2 | 3 | 4 | 5 | Avg. | StDev | (%) |
| S5-18-00 | 72 | 65 | 57 | 64 | 72 | 66 | 6.3 | 9.50 |
| S5-18-01 | 71 | 53 | 61 | 63 | 67 | 63 | 6.8 | 10.80 |
| S5-18-02 | 70 | 57 | 61 | 63 | 63 | 62.8 | 4.7 | 7.50 |
| S5-18-03 | 57 | 58 | 55 | 54 | 63 | 57.4 | 3.5 | 6.10 |
| S5-18-04 | 61 | 63 | 50 | 51 | 59 | 54.8 | 4.9 | 9.00 |
| S5-18-05 | 47 | 46 | 44 | 43 | 45 | 45 | 1.6 | 3.50 |
| S5-20-1 | 70 | 50 | 58 | 56 | 64 | 59.6 | 7.7 | 12.90 |
| S5-20-2 | 57 | 57 | 63 | 65 | 65 | 61.4 | 4.1 | 6.70 |
| S5-20-3A | 41 | 42 | 47 | 42 | 47 | 43.8 | 2.9 | 6.70 |
| S5-20-3B | 31 | 41 | 43 | 34 | 41 | 38 | 5.2 | 13.70 |
| S5-20-4 | 43 | 43 | 44 | 35 | 42 | 41.4 | 3.6 | 8.80 |
| S5-20-5 | 45 | 48 | 43 | 46 | 49 | 46.2 | 2.4 | 5.20 |

### Example 19: Exemplary Adhesive Base-Mechanically Reinforcing Composition (AB-MRC) Cured Mechanically Reinforced Adhesive Slabs

Slabs of adhesive base (AB) and mechanically reinforcing compositions (MRCs) were case following the method described elsewhere herein to evaluate the mechanical properties of adhesive dressings resulting from the combination of the two components. For these exemplary slabs, the MRC components were first mixed with inhibitor (F5-10-1) and then the AB composition was introduced. The slab compositions (Table 35) and average hardness values thereof (Table 36) are provided herein.

TABLE 35
Two-part AB-MRC slab compositions

| | | S5-22-02 | S5-22-06 | S5-22-07 | S5-22-08 |
|---|---|---|---|---|---|
| No. | Component | (g) | (g) | (g) | (g) |
| 1 | AB6-001 | 1.5 | 2 | 2 | 2 |
| 2 | MRC2-003 | 1.5 | | | |
| 3 | F5-18-01 | | 1 | | |
| 4 | F5-20-4 | | | 1 | |
| 5 | F5-20-5 | | | | 1 |
| 6 | F5-10-1 | 0.1 | 0.1 | 0.1 | 0.1 |

TABLE 36
Average shore 00 hardness values measured
for adhesive slab compositions

| Reading No. | S5-22-02 | S5-22-06 | S5-22-07 | S5-22-08 |
|---|---|---|---|---|
| 1 | 58 | 57 | 48 | 52 |
| 2 | 59 | 52 | 42 | 50 |
| 3 | 47 | 58 | 44 | 48 |
| 4 | 45 | 55 | 49 | 49 |
| 5 | 41 | 51 | 44 | 42 |
| Average | 50 | 55 | 45 | 48 |
| StDev | 8 | 3 | 3 | 4 |
| StDev (%) | 16 | 5 | 7 | 8 |

### Example 20: Tensile Properties of Exemplary Reinforced Adhesive Slabs

AB and MRC composite slabs were cast utilizing the methods described elsewhere herein (Table 37). Dogbone specimen were prepared for each slab in triplicate using an ASTM D412 C die and evaluated in a tensile testing machine (Instron, Norwood, MA) with a strain rate of 50 mm/min. The calculated Young's Moduli and % elongation (Bluehill® Universal, Instron, Norwood, MA) for each sample is provided in Table 38.

A substantial increase in the Young's Modulus for the dogbones comprising AB F5-37-1 demonstrated a synergistic effect of the AB and MRC2-003 compositions together, where the Young's Modulus increased four-fold when compared to crosslinking the AB with XL-11 alone (i.e., S5-39-5 vs S5-38-5 and S5-39-1, respectively).

TABLE 37
Exemplary slab compositions for tensile evaluation

| | Component (g) | | | | | | | | |
|---|---|---|---|---|---|---|---|---|---|
| | AB6- | F5- | | | | F5-10- | AB5- | AB2- | MRC |
| Formulation | 001 | 37-1 | XL 11 | XL 1B | D5^{e} | 1 | 001 | 004 | 2-003 |
| S5-38-5 | | 13.82 | 0.73 | | | 0.45 | | | |
| S5-38-6 | | 10.91 | | 3.64 | | 0.45 | | | |
| S5-38-7 | | | | | | 0.45 | | 7.5 | 7.5 |
| S5-38-8 | | | | | | 0.45 | 7.5 | | 7.5 |
| S5-38-9 | 7.5 | | | | | 0.45 | | | 7.5 |
| S5-39-1^{a} | | 13.85 | 0.815 | | 0.815 | 0.44 | | | |
| S5-39-2^{b} | | 14.54 | 0.07 | | 0.07 | 0.45 | | | |
| S5-39-3^{c} | | 10.92 | | 3.665 | 3.665 | 0.49 | | | |
| S5-39-4^{d} | | 14.2 | | 0.395 | 0.395 | 0.44 | | | |
| S5-39-5 | | 7.52 | | | | 0.44 | | | 6.15 |
| S5-39-7 | | 3.87 | | | | 0.49 | | | 6.11 |

^{a}mixture of XL 11 and D5 (1:1, 1.63 g);
^{b}mixture of XL 11 and D5 (1:1, 0.14 g);
^{c}mixture of XL 1B and D5 (1:1, 7.33 g);
^{d}mixture of XL 1B and D5 (1:1, 0.79 g);
^{e}D5 is decamethylcyclopentasiloxane (Andisil D5, AB specialty silicones).

TABLE 38
Tensile properties of exemplary slab compositions

| | | | | Young's | | |
|---|---|---|---|---|---|---|
| | | | Young's | Modulus | | Elongation |
| Sample | AB | XL | Modulus | StDev | Elongation | STDEV |
| S5-38-5 | F5-37-1 | XL-11 | 0.17 | 0.06 | 706% | 37% |
| S5-38-6 | F5-37-1 | XL-1B | 0.06 | 0.02 | 626% | 156% |
| S5-38-7 | AB2- | MRC2- | 0.23 | 0.02 | 421% | 118% |
| | 004 | 003 | | | | |
| S5-38-8 | AB5- | MRC2- | 0.46 | 0.08 | 569% | 85% |
| | 001 | 003 | | | | |
| S5-38-9 | AB6- | MRC2- | 0.37 | 0.13 | 447% | 144% |
| | 001 | 003 | | | | |
| S5-39-1 | F5-37-1 | XL-11 | 0.18 | 0.02 | 961% | 79% |
| S5-39-3 | F5-37-1 | XL-1B | 0.09 | 0.04 | 939% | 102% |
| S5-39-4 | F5-37-1 | XL-1B | 0.16 | 0.02 | 538% | 284% |
| S5-39-5 | F5-37-1 | MRC2- | 0.66 | 0.07 | 689% | 15% |
| | | 003 | | | | |
| S5-39-7 | F5-37-1 | MRC2- | 0.76 | 0.07 | 639% | 208% |
| | | 003 | | | | |

### Example 21: Evaluation of Vinyl and Filler Content on Reinforced Adhesive Tensile Properties

The contributions of vinyl siloxane(s) (VS80000) and fumed silica component(s) on the tensile properties of certain reinforced adhesives was evaluated based on the compositions described herein (Table 39), and their corresponding slab compositions (Table 40). The tensile properties of exemplary slab compositions are further provided herein (Table 41). Additionally, F5-37-2 is a dilution of the F5-10-1 inhibitor composition using D5 at a 1:1 ratio.

The calculated Young's Moduli and % elongation values were comparable for the four samples evaluated, given the spread in the standard deviations corresponding to the reported averages.

TABLE 39
AB compositions with varied filler and vinyl siloxane content

| | | F5- | F5- | | | F5- | |
|---|---|---|---|---|---|---|---|
| | | 41- | 41- | F5- | F5-41- | 41- | F5- |
| | | 2A-1 | 2A-2 | 42-1 | 2A-1 | 2A-2 | 42-1 |
| | | Mass | Mass | Mass | w/w | w/w | w/w |
| No. | Chemical | (g) | (g) | (g) | (%) | (%) | (%) |
| 1 | F5-41-1A | 35 | 35 | | 92% | 95% | 0% |
| 2 | AB6-001 | | | 70 | 0% | 0% | 91% |
| 3 | HMDS treated fumed silica, | 3 | 1.7 | 7 | 8% | 5% | 9% |
| | Cabosil TS530, Cabot | | | | | | |
| | Corporation | | | | | | |

TABLE 40
Exemplary slab compositions

| | | S5-39-6 | S5-45-7 | S5-45-8 | S5-45-9 |
|---|---|---|---|---|---|
| No. | Component | Mass (g) | Mass (g) | Mass (g) | Mass (g) |
| 1 | F5-37-1 | 9 | | | |
| 2 | F5-41-2A-1 | | 7.8 | | |
| 3 | F5-41-2A-2 | | | 9.1 | |
| 4 | F5-42-1 | | | | 9 |
| 5 | F5-10-1 | 0.45 | | | |
| 6 | F5-37-2 | | 0.5 | 0.6 | 0.6 |
| 7 | MRC2-003 | 9 | 7.8 | 9 | 9 |

TABLE 41
Tensile properties of certain AB compositions

| | Young's Modulus | STDEV | Elongation | STDEV |
|---|---|---|---|---|
| Sample Name | (MPa) | (MPa) | (%) | (%) |
| S5-39-6 | 0.47 | 0.11 | 291 | 121 |
| S5-45-7 | 0.46 | 0.04 | 311 | 38 |
| S5-45-8 | 0.45 | 0.04 | 349 | 83 |
| S5-45-9 | 0.43 | 0.06 | 275 | 30 |

### Example 22: Tensile Properly Evaluation of Exemplary Mechanically Reinforcing Compositions

Additional MRC compositions were examined to identify formulation variables which impact the tensile properties of cured slabs. The composition of MRC F5-57-1 and variant compositions thereof, and the corresponding slab compositions thereof, are provided herein (Tables 42a-42b; and Table 43), wherein the Young's Modulus and the percent elongation at break for each slab is further provided (Table 44).

The slab corresponding to MRC F5-57-2 achieved the highest Young's Modulus and percent elongation at break values, suggesting that VS80000 likely contributes to the observed optimization of tensile properties. While this finding is consistent with the increase in the Young's Moduli measured for slabs comprising variable AB compositions (e.g., each including approximately 35% VS80000) and MRC2-003 when compared to Young's Modulus of MRC2-003 alone, the synergistic enhancement in the Young's Modulus for the slab comprising AB F5-37-1 and MRC2-003 compared to the value achieved for the corresponding slab comprising AB5-001 suggest that the crosslinked telechelic composition of AB6-001 may be the primary contributor to the elevated Young's Modulus value observed herein.

TABLE 42a
F5-57-1 MRC composition

| No. | Chemical | Ref. | mmol/g | Mass (g) | w/w (%) |
|---|---|---|---|---|---|
| 1 | Pendant Silicon-Hydride Functional | H1 | 4.1 | 25 | 6.2% |
| | polydimethyl siloxane, trimethylsiloxy | | | | |
| | terminated (Andisil XL-11, AB Specialty | | | | |
| | Silicones) | | | | |
| 2 | Vinyl terminated dimethylpolysiloxane, | V1 | 0.121 | 148.6 | 36.6% |
| | 1000 cSt (Andisil VS1000, AB Specialty | | | | |
| | Silicones) | | | | |
| 3 | HMDS treated fumed silica (Cabosil TS530, | | | 58.8 | 14.5% |
| | Cabot Corporation) | | | | |
| 4 | Decamethylcyclopentasiloxane (Andisil | | | 173.4 | 42.7% |
| | D5, AB Specialty Silicones) | | | | |

TABLE 42b
Exemplary F5-57-1 MRC variant compositions

| | | | F5-57-2 | | F5-57-3 | | F5-57-4 | | F5-57-5 | |
|---|---|---|---|---|---|---|---|---|---|---|
| No. | Chemical | Ref. | (g) | w/w | (g) | w/w | (g) | w/w | (g) | w/w |
| 1 | F5-57-1 | | 20.1 | 80.7% | 20.2 | 92.6% | 20.2 | 92.2% | 20.2 | 86.3% |
| 2 | Vinyl Terminated | V2 | 3.6 | 14.5% | 1.2 | 5.5% | 0.0 | 0.0% | 0.0 | 0.0% |
| | Dimethylpolysiloxane, | | | | | | | | | |
| | 80000 cSt (Andisil | | | | | | | | | |
| | VS80000, AB Specialty | | | | | | | | | |
| | Silicones) | | | | | | | | | |
| 3 | Pendant Silicon-Hydride | H2 | | 0.0% | | 0.0% | 1.3 | 5.9% | 2.4 | 10.3% |
| | Functional polydimethyl | | | | | | | | | |
| | siloxane, trimethylsiloxy | | | | | | | | | |
| | terminated (Andisil XL-1B, | | | | | | | | | |
| | AB Specialty Silicones) | | | | | | | | | |
| 4 | HMDS treated fumed silica, | | 1.2 | 4.8% | 0.4 | 1.9% | 0.4 | 1.8% | 0.8 | 3.4% |
| | Cabosil TS530, Cabot | | | | | | | | | |
| | Corporation | | | | | | | | | |

TABLE 43
Exemplary slab compositions

| No. | Component | S5-58-1 | S5-58-2 | S5-58-3 | S5-58-4 | S5-58-5 |
|---|---|---|---|---|---|---|
| 1 | F5-57-1 | 2.40 | | | | |
| 2 | F5-57-2 | | 2.4 | | | |
| 3 | F5-57-3 | | | 2.4 | | |
| 4 | F5-57-4 | | | | 2.46 | |
| 5 | F5-57-5 | | | | | 2.41 |
| 6 | D5 | 1.42 | 1.42 | 1.42 | 1.43 | 1.43 |
| 7 | F5-10-1 | 0.16 | 0.16 | 0.16 | 0.16 | 0.16 |
| 8 | 3000 ppm Karstedt Catalyst | 0.01 | 0.01 | 0.01 | 0.01 | 0.01 |

TABLE 44
Tensile properties of MRC F5-57-1 and variants thereof

| | | AVERAGE of | | STDEV of |
|---|---|---|---|---|
| | AVERAGE | Tensile | STDEV | Tensile |
| | Young's | strain | Young's | strain |
| Sample | Modulus | at Break | Modulus | at Break |
| No. | [MPa] | [%] | [MPa] | [%] |
| S5-58-1 | 0.15 | 321 | 0.03 | 105 |
| S5-58-2 | 0.19 | 423 | 0.03 | 102 |
| S5-58-3 | 0.14 | 358 | 0.02 | 30 |
| S5-58-4 | 0.13 | 326 | 0.04 | 4 |
| S5-58-5 | 0.12 | 372 | 0.02 | 68 |

TABLE 45
Exemplary F5-57-1 variant compositions

| | | F5-62-1 | | F5-62-2 | | F5-66-1 | |
|---|---|---|---|---|---|---|---|
| No. | Chemical | (g) | w/w | (g) | w/w | (g) | w/w |
| 1 | F5-57-1 | 20 | 95% | 20 | 99% | 20 | 94% |
| 2 | Silica (Goddball G-6C, Iwase Cosfa) | 1 | 5% | 0 | 0% | 1 | 5% |
| 3 | Polydimethylsiloxane fluid, PMX-200, 5 | 0 | 0% | 0.2 | 1% | 0.2 | 1% |
| | cSt (Dow Chemical Company) | | | | | | |

The compositions provided in Table 29 and Table 45 were evaluated on either the outer calf or on the volar forearm position of subjects (volunteers 1 and 2; Vt1 and Vt2) following application of the following the application scheme provided in Table 46. One line-width amount of each of the adhesive base (AB) composition and the mechanically reinforcing composition (MRC) formulation was applied along the length of a stainless steel roller applicator (1.5 inch long; 0.5 inch diameter). The line width quantity applied using a needle nosed dispenser corresponds to a dose of approximately 1 mm diameter and 1.5 inches in length.

TABLE 46
AB and MRC composition variant in-use screening

| Volunteer (Vt) | No. | Site | Position | AB | MRC |
|---|---|---|---|---|---|
| 1 | 1 | Outer Calf | Upper Left | F5-78-1 | F5-66-1 |
| 1 | 2 | Outer Calf | Lower Left | F5-78-2 | F5-66-1 |
| 1 | 3 | Outer Calf | Upper Right | F5-78-2 | F5-66-1 |
| 1 | 4 | Outer Calf | Lower Right | F5-78-1 | F5-66-1 |
| 1 | 5 | Volar Forearm | Upper Left | F5-78-1 | F5-66-1 |
| 1 | 6 | Volar Forearm | Mid Left | F5-78-2 | F5-66-1 |
| 1 | 7 | Volar Forearm | Upper Right | F5-78-2 | F5-66-1 |
| 1 | 8 | Volar Forearm | Mid Right | F5-78-1 | F5-66-1 |
| 2 | 1 | Outer Calf | Upper Left | F5-78-1 | F5-66-1 |
| 2 | 2 | Outer Calf | Lower Left | F5-78-2 | F5-66-1 |
| 2 | 3 | Outer Calf | Upper Right | F5-78-2 | F5-66-1 |
| 2 | 4 | Outer Calf | Lower Right | F5-78-1 | F5-66-1 |
| 2 | 5 | Outer Calf | Lower Left | F5-78-2 | F5-66-1 |
| 2 | 6 | Outer Calf | Upper Right | F5-78-2 | F5-66-1 |
| 2 | 7 | Volar Forearm | Upper Right | F5-79-1 | F5-66-1 |

The AB was applied to a target skin area of approximately 4 to 6 square inches by rolling a uniform layer of formulation onto the target skin area. The MRC was then applied over the AB coated skin area using the roller to form the crosslinked adhesive.

36 hours after product application, Volunteer 1 (Vt1) site 6 (i.e., volar forearm; mid left) was identified as the best performer, with >90% of the adhesive area remaining intact, while Vt1 sites 1 through 4 remained 80% intact.

Volunteer 2 (Vt2) sites 1 and 3 were resistant to removal following mechanical rubbing. The site 3 adhesive was removed in 1 continuous piece. Reapplication of F5-78-2 to the outer calf areas confirmed the durability robustness of the AB-MRC composition corresponding to Vt2 sites 5 and 6 following overnight wear, with 95% of cured adhesive remaining intact.

Vt2 site 7 did not produce as mechanically robust an adhesive following 5 minutes of cure, compared to the other sites

### Example 23: Emulsions Comprising Crosslinked Telechelic Siloxanes

An emulsion composition comprising the crosslinked telechelic siloxane in F5-37-1 was prepared as follows. Phase A ingredients were mixed for 5 minutes at 500 rpm with an overhead mixer (IKA Eurostar 60) and a Jiffy blade. Phase B ingredients were similarly mixed. The mixed Phase B was slowly introduced to the Phase A mixture while mixing at 700 rpm to form an emulsion. The emulsion was mixed for 30 minutes at 500 rpm.

An aliquot of F5-67-1 (Table 47), approximating 1/10 of the length of a 1.5 inch roller, was dispensed onto a metal roller and applied to a 1 square inch (1 in²) area of volar forearm skin. AB and MRC formulations F5-64-1 and F5-66-1 were applied, using one roller length dose of each formulation, over the emulsion to create a modified adhesive skin dressing covering a 2 inch by 2 inch area. Following overnight wear, at 16 hours, the dressing was evaluated and remained 95% intact. The emulsion was compatible with the telechelic composition and may be modified to incorporate any of a number of additional skin beneficial ingredients including but not limited to vitamins, extracts, and other cosmetic skin actives known to those of ordinary skill in the art.

TABLE 47
F5-67-1 composition (anhydrous lotion)

| | | Mass | w/w |
|---|---|---|---|
| No. | Chemical Name | (g) | (%) |
| 1 | Cetyl diglyceryl tris(trimethyl- | 0.56 | 1.1% |
| | siloxy)silylethyl dimethicone | | |
| | (DOWSIL ™ ES-5600 Silicone | | |
| | Glycerol Emulsifier) | | |
| 2 | Caprylyl Methicone (DOWSIL ™ | 2.4 | 4.8% |
| | FZ-3196 Fluid) | | |
| 3 | Dimethicone (XIAMETER ™ PMX-200 | 4.4 | 8.8% |
| | Silicone Fluid 5 cSt) | | |
| 4 | F5-37-1 | 4.8 | 9.6% |
| 5 | 1,3-butylene glycol (Jeechem BUGL) | 18.9 | 37.8% |
| 6 | Glycerin | 18.9 | 37.8% |

Phase A: components 1-4; Phase B: components 5-6.

### Example 24: Chemical Irritancy Protection (In-Use Study No. CS5-69-1)

In one aspect, the present disclosure describes an evaluation of the feasibility of the use of an exemplary adhesive dressing to protect the skin from exposure to a model chemical irritant.

Sodium dodecyl sulfate (SDS) with greater than 99.0% purity as determined by gas chromatography (pcode 102539087, STBK8835) was purchased from Sigma Aldrich (St. Louis, MO). Solutions of SDS (i.e., 1% and 0.5% w/w solutions) were prepared in reverse osmosis (RO) water and allowed to dissolve until a clear solution was visualized.

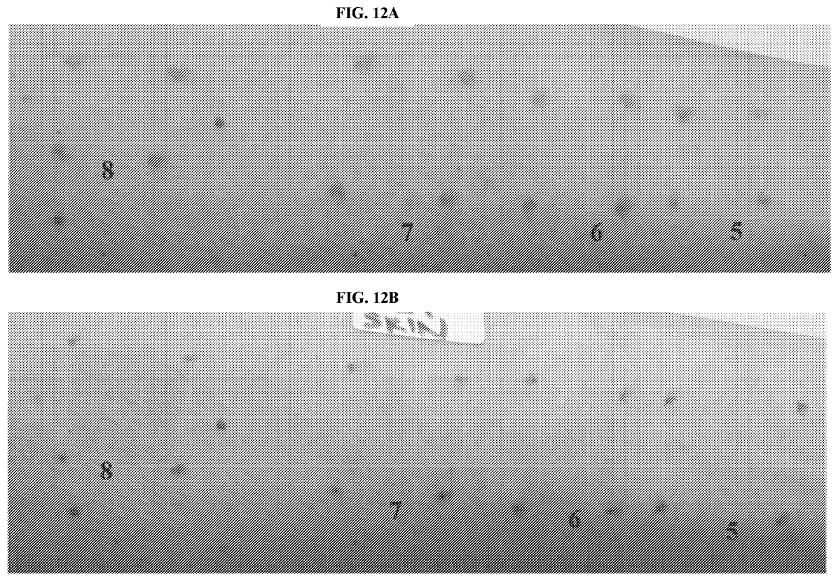

The volar forearm skin of a volunteer was then rinsed with water and dabbed to remove excess moisture. Eight target skin application sites with areas of approximately 1 square inch (1 in²) were demarcated using a marker. A board-certified dermatologist provided baseline erythema and dryness scoring for the eight sites. Baseline photographs of the sites were then taken (FIGS. 11A-11B and FIGS. 12A-12B).

Grade CFP2 filter papers (Grainger, Lake Forest, IL) were folded into 1 inch square areas and used as the reservoirs for the SDS solutions. The folded filter paper was placed on the adhesive side of a of piece gentle paper tape (3M Nexcare, St. Paul, MN), and 0.3 mL of the designated SDS solution was then dispensed onto each filter. With the exception of sites 3 and 7, the loaded filter papers were then applied to the skin sites following the scheme provided herein (Table 48), where the ascending site number for each volar forearm reflects an increasing proximity to the elbow from the wrist position.

TABLE 48
Applicant scheme

| Site No. | Left Arm | Site No. | Right Arm |
|---|---|---|---|
| 1 (wrist) | 0.5% | 5 | 1.0% |
| 2 | 0.5% | 6 | 1.0% |
| 3 | AB/MRC (1 hour) 1.0% | 7 | AB/MRC (1 hour) 1.0% |
| 4 (elbow) | 0.5% | 8 | 1.0% |

For those two skin sites, 4 square inch areas of the AB/MRC combination (AB F5-64-1/MRC F5-66-1) were applied to each skin site as follows. The AB composition, representing a consumer dispensed quantity, approximating a 1.5 mm wide line was applied to the length of a 1.5 inch long roller having a 0.5 inch diameter, and rolled over the target area to lay down a uniform coating of formulation. An equivalent amount of the MRC formulations was then applied to another roller and applied over the AB layer on the skin. One hour after application, the designated SDS solutions were applied over the cured AB/MRC layer. A final strip of the gentle paper tape was applied across the length of the forearm and perpendicular to the individual tape strips to secure the SDS loaded filters. Sites 1, 2 and 4 received another 0.3 mL dose of 0.5% SDS after two hours of exposure.

Following 15 hours of SDS exposure, the filter papers at each of the eight sites were removed. The sites were graded using the scale described in Table 49 at one hour and six hours after removal. The skin treatment sites were remarked after grading was performed one hour post patch removal.

TABLE 49
Grading scheme

| | |
|---|---|
| Score | Observation |

Erythema Scale (5 point)

| | |
|---|---|
| 0 | no visible reaction |
| 0.5 | slight patchy erythema |
| 1 | moderate, uniform erythema, possibly slight infiltration |
| 2 | intense erythema, possibly infiltration and/ or vesicles |
| 3 | fiery erythema with oedema, infiltration, |
| | vesicles, possibly bullae |

Scaling/Dryness

| | |
|---|---|
| 0 | none |
| 1 | weak |
| 2 | moderate |
| 3 | large flakes |
| 4 | intense peeling |

Evaluation conducted by board certified dermatologist.

A summary of the live grading results (Tables 50a-50b and Table 51) show that sites 3 and 7, where the product was applied prior to SDS exposure, did not change from the baseline score, whereas the unprotected sites each showed a change in either the erythema and/or dryness scores at 6 hours following SDS patch removal. For the 0.5% SDS unprotected sites, the erythema score increased from 3 scores of 0 to 3 scores of 1 and the dryness score increased for 3 scores of 0 to 2 scores of 1. For the 1% SDS unprotected sites, the erythema score increased from 3 scores of 0 to 2 scores of 0.5 and 1 score of 1. For these sites, the dryness also increased from 3 scores of 0 to 2 grades of 1.

The photographs corresponding baseline and 6 hours after SDS patch removal are shown in FIGS. 11A-11B and FIGS. 12A-12B, for the left and the right volar forearms, respectively. The assigned site number corresponding to the application scheme is also indicated in FIGS. 11A-11B and FIGS. 12A-12B.

TABLE 50a
Erythema live visual grade

| | |
|---|---|
| | Frequency per |
| SDS | Timepoint (hr) |

| Treatment | Erythema | 0 | 16 | 21 |
|---|---|---|---|---|
| 0.5% | 0 | 3 | 3 | |
| | 1 | | | 3 |
| 1% | 0 | 3 | 1 | |
| | 0.5 | | | 2 |
| | 1 | | 2 | 1 |
| 1% over product | 0 | 2 | 2 | 2 |

TABLE 50b
Dryness live visual grade

| | | |
|---|---|---|
| | | Frequency per |
| SDS | Dryness | Timepoint (hr) |

| Treatment | Grade | 0 | 16 | 21 |
|---|---|---|---|---|
| 0.5% | 0 | 3 | 1 | 1 |
| | 1 | | 2 | 2 |
| 1% | 0 | 3 | | 1 |
| | 1 | | 3 | 2 |
| 1% over product | 0 | 2 | 2 | 2 |

### Example 25: Clinical Evaluation of Transepidermal Water Loss (TEWL) in Healthy Volunteers

Two skin adhesive dressing compositions (Table 51) were evaluated in a group of 8 female volunteers who were 18-35 years of age (inclusive) and Fitzpatrick skin phototypes I and II. In addition, the volunteers were required to have mild to moderate dryness on the outer calf sites (a score of 3-4 on a 10-point Visual Dryness scale (Table 52) as determined by the Expert Grader (EG). Good Clinical Practices in compliance with International Conference on Harmonization guidelines were implemented for the study.

Briefly, two (2) test sites on the outer calves of with one (1) site on each leg, two (2) test sites on the arms with one (1) site on each antecubital fossa (ACF), and one (1) additional non-treated region on one (1) arm and one (1) outer calf were used as controls, for a total of six (6) total assessed sites. Each of the four (4) test sites were treated with one (1) of two (2) different Investigational Products (IPs) with each IP applied to one (1) outer calf test site and one (1) antecubital fossa test site in a randomized fashion.

The AB and the corresponding MRC doses of 0.34+/−0.01 g and 0.4+/−0.01 g were applied uniformly over the designated 3 inch by 4 inch skin area using separate roller applicators by layering the MRC over the AB. Prior to each measurement, the volunteer was acclimated for a minimum of 25 minutes in an environmentally controlled chamber. The Cortex Technology DermaLab® TEWL X (Aalborg, Denmark) was used to measure Trans-Epidermal Water Loss in accordance with the manual.

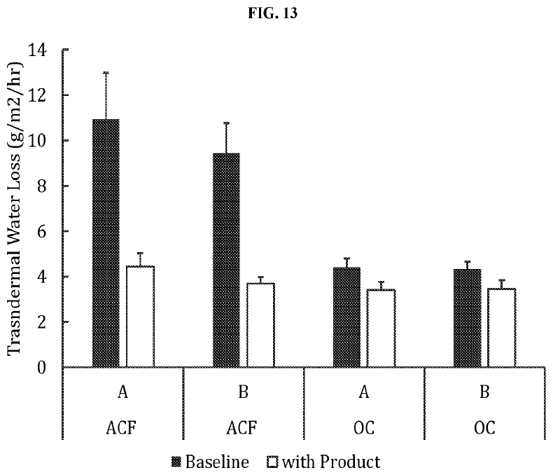

FIG. 13 shows the average TEWL value measured for each formulation at the ACF and OC regions, for eight and seven volunteers, respectively, at baseline and one-hour after product application. The error bars indicate the standard error. The percentage change in TEWL one hour following the application of products A and B is summarized in Table 53. The statistical significance of the changes measured in the TEWL value was determined using the Student paired t-test. For the outer calf locations, product B trended toward significance with a p value of 0.11, whereas the 3 other sites each showed p values less than 0.005, demonstrating significant decreases in TEWL were achieved due to product application.

For eight volunteers, the expert grader visual dry skin grading at baseline and at 48 hours after product wear is provided in Table 54. The Student's paired t-test was applied to demonstrate significant improvements in the visual dryness for the combined treatment sites when compared to their baseline values (p=0.01).

TABLE 51
Clinical study product compositions

| Product | AB | MRC |
|---|---|---|
| A | AB2-004 | MRC2-003 |
| B | AB4-002 | MRC2-003 |

TABLE 52
Visual dryness scale

| Scale Value | Observation |
|---|---|
| 0 | None |
| 1- | Mild - slight flaking, no scaling |
| 4- | Moderate - obvious flaking, some scaling |
| 7- | Severe - Marked scaling, slight to |
| | prominent fissuring |

TABLE 53
Percent change in TEWL one hour following product application

| Site | antecubital fossa (ACF) | | Outer Calf (OC) | |
|---|---|---|---|---|
| Formulation | A* | B* | A* | B |
| Change in TEWL | −59% | −61% | −23% | −20% |

*p < 0.005.

TABLE 54
Expert grader visual dry skin scores before and after product wear

| Timepoint | Product | SE | Control | SE |
|---|---|---|---|---|
| Baseline | 3.6 | 0.14 | 3.6 | 0.14 |
| 48 hours | 2.9 | 0.22 | 3.7 | 0.13 |
| p-value | 0.01 | | n.s. | |

### Example 26: Primary Irritation Study

A primary irritation study was conducted to evaluate the potential of an exemplary adhesive skin dressing to induce irritant contact dermatitis. Fifty-two (52) healthy volunteers aged 22-79, inclusive, participated in the study. Approximately 0.2 mL of AB2-002 was applied to the designated skin site located between the beltline and the shoulder blade, lateral to the midline of the back. MRC2-002 (0.2 mL) was applied to a semi-occlusive patch comprising 1 square inch of absorbent pad centered on a translucent perforated adhesive strip. The MRC on the loaded patch was placed over the corresponding AB2-002 treated skin site to form the crosslinked skin adhesive dressing. The patch covering the test product was removed after 48 hours (Day 2) of continuous product exposure and evaluated by an expert grader using the Erythemal Scoring Scale provided in Table 55. The skin sites were reevaluated on Day 3, or 24 hours after patch removal. For the 52 volunteers, no visible erythema, corresponding to a score of 0, was observed on either Day 2 or Day 3 of the study (Table 56).

TABLE 55
Erythemal scoring scale

| Score | Description |
|---|---|
| 0 | No visible erythema |
| 0.5 | Slight, barely perceptible erythema |
| 1 | Mild erythema |
| 2 | Moderate erythema |
| 3 | Marked erythema |
| 4 | Severe erythema |

TABLE 56
Primary irritation study results

Frequency

| Score | Day 2 | Day 3 |
|---|---|---|
| 0 | 52 | 52 |
| 0.5 | 0 | 0 |
| 1 | 0 | 0 |
| 2 | 0 | 0 |
| 3 | 0 | 0 |
| 4 | 0 | 0 |

### Example 27: In-Use Testing Evaluating 48-Hour Durability

Exemplary adhesive skin dressing combinations of AB and MRC were evaluated in a consumer use scenario where a band of each formulation was dispensed along the length of separate application rollers and then sequentially applied to each skin area, wherein the MRC applied over the AB layer. Table 57 provides a summary of the combinations evaluated and their qualitative performance over a 48-hour period of continuous wear. The 48-hour durability grade is a semi-quantitative assessment of the total area of adherent skin dressing remaining at the end of the evaluation period. The target in-use sites included three different skin sites along the volar forearm (VF), referred to as wrist (w), center (c) and elbow (e), based on their position relative to those anatomic locations. For the outer calf (OC), the upper (u) and the lower (1) areas were evaluated. The area of application was either a 2×2 inch square or a 2×3 inch rectangular area. The volunteer was allowed to engage in standard daily routines, including exercise and bathing. For the combinations evaluated, good durability was observed over the 48 hour wear period, with all of the sites achieving 80% durability or above and a majority of the sites achieving 95% durability.

TABLE 57
Summary of AB and MRC combinations evaluated in-use

| | | | 48-hour | Skin | Area |
|---|---|---|---|---|---|
| No. | AB | MRC | durability | site | (in × in) |
| 1 | F5-16-01 | MRC2-003 | 85% | VF(w) | 2 × 3 |
| 2 | F5-16-01 | MRC2-003 | 95% | VF(c) | 2 × 2 |
| 3 | F5-16-01 | MRC2-003 | 95% | VF(w) | 2 × 2 |
| 4 | F5-16-01 | MRC2-003 | 85% | VF(c) | 2 × 3 |
| 5 | AB7-001 | MRC2-003 | 80% | VF(w) | 2 × 2 |
| 6 | AB7-001 | F5-57-1 | 90% | VF(c) | 2 × 2 |
| 7 | AB7-001 | F5-62-1 | 95% | VF(e) | 2 × 2 |
| 8 | AB7-001 | F5-57-1 | 80% | VF(w) | 2 × 2 |
| 9 | AB7-001 | F5-62-2 | 90% | VF(c) | 2 × 2 |
| 10 | AB7-001 | F5-62-1 | 95% | VF(e) | 2 × 2 |
| 11 | F5-37-1 | F5-66-1 | 95% | OC (u) | 2 × 2 |
| 12 | F5-64-1 | F5-62-2 | 95% | OC(l) | 2 × 2 |
| 13 | F5-64-1 | F5-66-1 | 95% | OC(u) | 2 × 2 |
| 14 | F5-37-1 | F5-62-2 | 95% | OC(l) | 2 × 2 |

### Example 28: Ancillary Product Formulation

The present disclosure further provides non-limiting emulsion compositions which comprise the crosslinked telechelic compositions of the present disclosure, including exemplary emulsion F6-67-1 (Table 58). In certain embodiments, the emulsions may comprise one or more humectants for skin moisturization (e.g., glycerin). In certain embodiments, the emulsions may comprise one or more cosmetic ingredients which provide a skin benefit, including but not limited to vitamins, antioxidants, and extracts.

In certain embodiments, non-limiting emulsions of the present disclosure may comprise at least one cross-linked telechelic, at least one emulsifier, and at least one polar and/or water-miscible/soluble solvent. In certain embodiments, the at least one polar and/or water-miscible/soluble solvent is present in a separate phase from the silicon phrase of the emulsion.

Non-limiting exemplary emulsifiers include lauryl PEG/PPG-18/18 Methicone, PEG-12 dimethicone cross-polymer, PEG/PPG-18/18 dimethicone, PEG/PPG-19/19 dimethicone, PEG/PPG-19/19 dimethicone and C13-16 isoparaffin and C10-13 isoparaffin, PEG-10 Dimethicone, Bis-isobutyl PEG/PPG-10/7 dimethicone copolymer, dimethicone and PEG/PPG-18/18 dimethicone, PEG-11 methyl ether dimethicone, PEG/PPG-20/22 butyl ether dimethicone, PEG-3 dimethicone, PEG-10 dimethicone, PEG-32 methyl ether dimethicone, PEG-9 polydimethylsiloxyethyl dimethicone, lauryl PEG-9 polydimethylsiloxyethyl dimethicone, polyglyceryl-3-disiloxane dimethicone, and polyglyceryl-3-polydimethylsiloxyethyl dimethicone.

The polar, water soluble solvent consisting the second emulsion phase may include water, glycerin, propylene glycol, butylene glycol, and pentylene glycol, inter alia.

TABLE 58
Exemplary emulsion formulation F5-67-1

| No. | Chemical Name | w/w (%) |
|---|---|---|
| 1 | Cetyl Diglyceryl Tris(Trimethyl- | 1.1% |
| | siloxy)silylethyl Dimethicone | |
| | (DOWSIL ™ ES-5600 Silicone Glycerol Emulsifier) | |
| 2 | Caprylyl Methicone (DOWSIL ™ FZ-3196 Fluid) | 4.8% |
| 3 | Dimethicone (XIAMETER ™ PMX-200 | 8.8% |
| | Silicone Fluid 5 cSt) | |
| 4 | F5-37-1 | 9.6% |
| 5 | 1,3 Butylene Glycol (Jeechem BUGL) | 37.8% |
| 6 | Glycerin | 37.8% |

### ENUMERATED EMBODIMENTS

The following exemplary embodiments are provided, the numbering of which is not to be construed as designating levels of importance:

Embodiment 1 provides a telechelic polymer composition comprising a cross-linked reaction product of any of:
(i) at least one polysiloxane (a) comprising a number of diorganosiloxy monomers, at least one 1-alkenyl-1-organosiloxy monomer, and two termini, wherein each terminus is independently selected from a triorganosiloxy group and a 1,1-organo-1-alkenyl-siloxy group, and wherein at least three Si atoms present in the polysiloxane are alkenyl-substituted;
(ii) at least one polysiloxane (b) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H);
(iii) at least one polysiloxane (c) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted;
(iv) at least one inert formulation-compatible polysiloxane (d); and
(v) at least one Group X transition metal catalyst.

Embodiment 2 provides the composition of Embodiment 1, further comprising:
(vi) at least one additional polysiloxane (f), wherein the at least one additional polysiloxane comprises a number of diorganosiloxy monomers, optionally a 1,1-diorgano-hydrosiloxy monomer, and two termini, wherein each terminus is independently selected from a triorganosiloxy group and a 1,1-diorgano-hydrosiloxy group, and wherein one Si atom present in the polysiloxane is substituted with H (i.e., Si—H).

Embodiment 3 provides the composition of Embodiment 1 or 2, wherein the at least one polysiloxane (a) possesses at least one of the following properties:
(i) a viscosity ranging from about 800 cSt to about 1200 cSt;
(ii) an average molecular weight ranging from about 15 kDa to about 45 kDa; and
(iii) an alkenyl equivalent per kilogram ranging from about 0.01 to about 0.70.

Embodiment 4 provides the composition of any one of Embodiments 1-3, wherein the at least one polysiloxane (b) possesses at least one of the following properties:
(i) a viscosity of about 10,000 cSt;
(ii) an average molecular weight ranging from about 45 kDa to about 75 kDa; and
(iii) a hydride weight percent of about 0.0010% to about 0.010%.

Embodiment 5 provides the composition of any one of Embodiments 1-4, wherein the at least one polysiloxane (b) possesses at least one of the following properties:
(i) a viscosity of about 7 cSt to about 10 cSt;
(ii) an average molecular weight of about 1 kDa to about 1.1 kDa; and
(iii) a hydride weight percent of about 0.18% to about 0.20%.

Embodiment 6 provides the composition of any one of Embodiments 1-5, wherein the at least one polysiloxane (c) possesses at least one of the following properties:
(i) a viscosity ranging from about 5,000 cSt to about 170,000 cSt; and
(ii) a vinyl equivalent per kilogram ranging from about 0.010 to about 0.040.

Embodiment 7 provides the composition of any one of Embodiments 2-6, wherein the at least one polysiloxane (f) possesses at least one of the following properties:
(i) a viscosity ranging from about 150 cSt to about 250 cSt;
(ii) an average molecular weight ranging from about 5 kDa to about 15 kDa; and
(iii) a weight fraction ranging from about 0.01% to about 75% of the reactant composition.

Embodiment 8 provides the composition of any one of Embodiments 1-7, wherein the at least one polysiloxane (a) is a compound of formula (Ia):

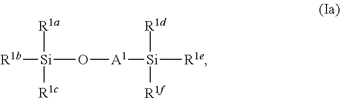

wherein:
A¹ comprises m units of

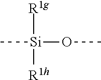

monomer and n units of

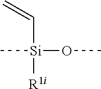

monomer,
   wherein each ---bond is a Si—O bond;
m is an integer ranging from 410 to 470;
n is an integer ranging from 1 to 50;
R^{1a}, R^{1b}, R^{1c}, R^{1d}, R^{1e}, and R^{1f} are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, optionally substituted C₂-C₁₂ heteroaryl, and optionally substituted C₂-C₆ alkenyl,
   wherein R^{1a}, R^{1b}, R^{1c}, R^{1d}, R^{1e}, and R^{1f} are selected such that each Si atom is substituted with no more than one optionally substituted C₂-C₆ alkenyl; and
R^{1g}, R^{1h}, and R¹ⁱ are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl.

Embodiment 9 provides the composition of Embodiment 8, wherein R^{1a}, R^{1b}, R^{1c}, R^{1d}, R^{1e}, R^{1f}, R^{1g}, R^{1h}, R¹ⁱ, m, and n are selected such that the at least one polysiloxane in (a) possesses at least one of the following properties:
(i) a viscosity ranging from about 800 cSt to about 1200 cSt;
(ii) an average molecular weight ranging from about 15 kDa to about 45 kDa; and
(iii) an alkenyl equivalent per kilogram ranging from about 0.01 to about 0.70.

Embodiment 10 provides the composition of any one of Embodiments 1-9, wherein the at least one polysiloxane in (b) is a compound of formula (Ib):

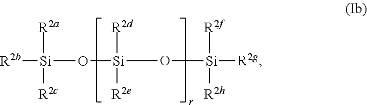

wherein:
R^{2a}, R^{2b}, R^{2c}, R^{2f}, R^{2g}, and R^{2h} are each independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
each occurrence of R^{2d} and R^{2e} is independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
o is an integer ranging from 500 to 1500; and
wherein R^{2a}, R^{2b}, R^{2c}, R^{2d}, R^{2e}, R^{2f}, R^{2g}, and R^{2h} are selected such that each Si atom is substituted with no more than one H atom.

Embodiment 11 provides the composition of Embodiment 10, wherein R^{2a}, R^{2b}, R^{2c}, R^{2d}, R^{2e}, R^{2f}, R^{2g}, R^{2h}, and o are selected such that the at least one polysiloxane in (b) possesses at least one of the following properties:
(i) a viscosity of about 10,000 cSt;
(ii) an average molecular weight ranging from about 45 kDa to about 75 kDa; and
(iii) a hydride weight percent of about 0.001% to about 0.010%.

Embodiment 12 provides the composition of Embodiment 11, wherein R^{2a}, R^{2b}, R^{2e}, R^{2d}, R^{2e}, R^{2f}, R^{2g}, R^{2h}, and o are selected such that the at least one polysiloxane in (b) possesses at least one of the following properties:
(i) a viscosity of about 7 cSt to about 10 cSt;
(ii) an average molecular weight ranging from about 1 kDa to about 1.1 kDa; and
(iii) a hydride weight percent of about 0.18% to about 0.20%.

Embodiment 13 provides the composition of any one of Embodiments 1-12, wherein the at least one polysiloxane in (c) is a compound of formula (Ic):

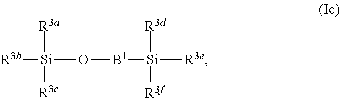

wherein:
B¹ comprises p units of

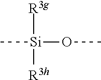

monomer and q units of

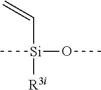

monomer,
   wherein each ---- bond is a Si—O bond;
R^{3a}, R^{3b}, R^{3c}, R^{3d}, R^{3e}, and R^{3f} are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, optionally substituted C₂-C₁₂ heteroaryl, and optionally substituted C₂-C₆ alkenyl,
   wherein R^{3a}, R^{3b}, R^{3c}, R^{3d}, R^{3e}, and R^{3f} are selected such that each Si atom is substituted with no more than one C₂-C₆ alkenyl;
R^{3g}, R^{3h}, and R³ⁱ are each independently C₁-C₆ alkyl;
p is an integer ranging from 500 to 2000; and
q is an integer ranging from 0 to 50.

Embodiment 14 provides the composition of Embodiment 13, wherein R^{3a}, R^{3b}, R^{3c}R^{3d}, R^{3e}, R^{3f}, p, and q, are selected such that the at least one polysiloxane in (c) possesses at least one of the following properties:
(i) a viscosity ranging from about 5,000 cSt to about 170,000 cSt; and
(ii) an alkenyl equivalent per kilogram ranging from about 0.010 to about 0.040.

Embodiment 15 provides the composition of any one of Embodiments 2-14, wherein the at least one polysiloxane in (f) is a compound of formula (If):

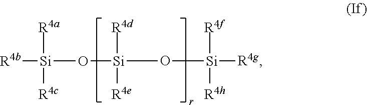

wherein:
R^{4a}, R^{4b}, R^{4c}, R^{4f}, and R^{4g} are each independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
each occurrence of R^{4d} and R^{4e} is independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
r is an integer ranging from 100 to 400; and
wherein no more than one of R^{4a}, R^{4b}, R^{4c}, R^{4d}, R^{4e}, R^{4f}, R^{4g}, and R^{4h} is H.

Embodiment 16 provides the composition of Embodiment 15, wherein R^{4a}, R^{4b}, R^{4c}, R^{4d}, R^{4e}, R^{4f}, R^{4g}, R^{4h}, and r are selected such that the at least one polysiloxane in (f) possesses at least one of the following properties:
(i) a viscosity ranging from about 150 cSt to about 250 cSt;
(ii) an average molecular weight ranging from about 5 kDa to about 15 kDa; and
(iii) a weight fraction ranging from about 0.01% to about 75% of the reactant composition.

Embodiment 17 provides the composition of any one of Embodiments 1-16, wherein the Group X catalyst comprises Pt.

Embodiment 18 provides the composition of Embodiment 17, wherein the Pt is Pt(0).

Embodiment 19 provides the composition of any one of Embodiments 1-18, wherein the Group X catalyst is Karstedt's catalyst:

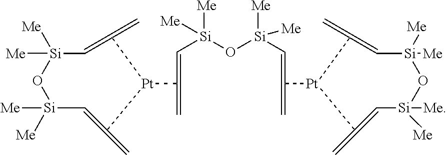

Embodiment 20 provides the composition of any one of Embodiments 1-19, wherein the inert formulation-compatible polysiloxane is a compound of formula (Id):

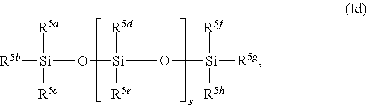

wherein:
R^{5a}, R^{5b}, R^{5c}, R^{5d}, R^{5e}, R^{5f}, R^{5g}, and R^{5h} are each independently C₁-C₆ alkyl; and
s is an integer ranging from 1 to about 500.

Embodiment 21 provides the composition of any one of Embodiments 1-20, wherein the inert formulation-compatible polysiloxane is selected from the group consisting of polydimethylsiloxane, dimethiconol, disiloxane, trisiloxane, and diphenyl dimethicone/vinyl diphenyl dimethicone/silsesquioxane cross-polymer.

Embodiment 22 provides the composition of any one of Embodiments 1-19 and 21, wherein the inert formulation-compatible polysiloxane is decamethylcyclopentasiloxane.

Embodiment 23 provides the composition of any one of Embodiments 1-22, wherein at least one of the following applies:
(i) R^{1a}, R^{1b}, R^{1c}, R^{1d}, R^{1e}, R^{1f}, R^{1g}, R^{1h}, and R¹ⁱ are each independently CH₃ or CH═CH₂;
(ii) R^{2a}, R^{2b}, R^{2c}, R^{2d}, R^{2e}, R^{2f}, R^{2g}, and R^{2h} are each independently H or CH₃;
(iii) R^{3a}, R^{3b}, R^{3c}, R^{3d}, R^{3e}, and R^{3f} are each independently CH₃ or CH═CH₂;
(iv) R^{3g}, R^{3h}, and R³ⁱ are each independently CH₃ or CH═CH₂;
(v) R^{4a}, R^{4b}, R^{4c}, R^{4d}, R^{4e}, R^{4f}, R^{4g}, and R^{4h} are each independently H or CH₃; and
(vi) R^{5a}, R^{5b}, R^{5c}, R^{5d}, R^{5e}, R^{5f}, R^{5g}, and R^{5h} are each independently CH₃.

Embodiment 24 provides the composition of any one of Embodiments 1-23, wherein the composition further comprises at least one additive.

Embodiment 25 provides the composition of Embodiment 24, wherein the at least one additive is at least one selected from the group consisting of glycerin, cetyl diglyceryl tris(trimethylsiloxy)silylethyl dimethicone, hexamethyldisilazane (HMDS) fumed silica, and polyoxyethylene/polyoxypropylene copolymer (PEG/PPG-18/18 dimethicone).

Embodiment 26 provides the composition of any one of Embodiments 1-25, wherein the composition has a viscosity ranging from about 4,000 cSt to about 100,000 cSt.

Embodiment 27 provides the composition of any one of Embodiments 1-26, wherein one of the following applies:
(a) the composition has a viscosity ranging from about 4,000 cSt to about 8,000 cSt; or
(b) the composition has a viscosity ranging from about 30,000 to about 50,000 cSt.

Embodiment 28 provides the composition of any one of Embodiments 1-27, wherein the composition has a total ratio of units of silicon hydride (i.e., Si—H) to vinyl-substituted silicon (i.e., Si—C(H)═CH₂) in all reactant components ranging from about 0.01 to about 0.8.

Embodiment 29 provides the composition of any one of Embodiments 1-28, wherein the composition has a total ratio of units of silicon hydride (i.e., Si—H) to vinyl-substituted silicon (i.e., Si—C(H)═CH₂) in all polysiloxane reactant components ranging from about 0.4 to about 0.6.

Embodiment 30 provides the composition of any one of Embodiments 1-29, wherein at least one of the following applies.
(i) the at least one polysiloxane in (a) comprises trimethylsiloxy terminated, 0.8-1.2% vinylmethylsiloxane dimethylsiloxane copolymer;
(ii) the at least one polysiloxane in (b) comprises hydride terminated polydimethylsiloxane;
(iii) the at least one polysiloxane in (c) comprises vinyl terminated polydimethylpolysiloxane;
(iv) the at least one inert formulation-compatible polysiloxane comprises polydimethyl siloxane and/or decamethylcyclopentasiloxane;
(v) the at least one Group X transition metal catalyst comprises Karstedt's catalyst; and
(vi) the at least one additional polysiloxane in (f) comprises monohydride terminated polydimethylpolysiloxane.

Embodiment 31 provides the composition of any one of Embodiments 1-30, wherein at least one of the following applies:
(i) the at least one polysiloxane in (a) comprises about 6.0% to about 12.0% of the composition by weight (w/w %);
(ii) the at least one polysiloxane in (b) comprises about 6.0% to about 12.0% of the composition by weight (w/w %);
(iii) the at least one polysiloxane in (c) comprises about 20.0% to about 30.0% of the composition by weight (w/w %);
(iv) the at least one formulation-compatible polysiloxane comprises about 10.0% to about 60.0% of the composition by weight (w/w %);
(v) the at least one Group X catalyst comprises about 0.0010% (10 ppm) to about 0.02% (200 ppm) of the composition by weight (w/w %); and
(vi) the at least one additional polysiloxane in (f) comprises about 1.0% to about 10.0% of the composition by weight (w/w %).

Embodiment 32 provides the composition of any one of Embodiments 1-31, wherein at least one of the following applies:
(i) the at least one polysiloxane in (a) comprises about 9.0% of the composition by weight (w/w %);
(ii) the at least one polysiloxane in (b) comprises about 9.4% of the composition by weight (w/w %);
(iii) the at least one polysiloxane in (c) comprises about 24.2% of the composition by weight (w/w %);
(iv) the at least one formulation-compatible polysiloxane comprises about 49% of the composition by weight (w/w %);
(v) the at least one Group X catalyst comprises about 0.0048% (48 ppm) of the composition by weight (w/w %); and
(vi) the at least one additional polysiloxane in (f) comprises about 5.8% of the composition by weight (w/w %).

Embodiment 33 provides the composition of any one of Embodiments 1-30, wherein at least one of the following applies:
(i) the at least one polysiloxane in (a) comprises about 14.0% to about 20.0% of the composition by weight (w/w %);
(ii) the at least one polysiloxane in (b) comprises about 0.20% to about 0.40% of the composition by weight (w/w %);
(iii) the at least one polysiloxane in (c) comprises about 18% to about 40% of the composition by weight (w/w %);
(iv) the at least one formulation-compatible polysiloxane comprises about 35% to about 50% of the composition by weight (w/w %);
(v) the at least one Group X catalyst comprises about 0.0080% (80 ppm) to about 0.0120 (120 ppm) of the composition by weight (w/w %); and
(vi) the at least one additional polysiloxane in (f) comprises about 7% to about 12% of the composition by weight (w/w %).

Embodiment 34 provides the composition of any one of Embodiments 1-30 and 33, wherein at least one of the following applies:
(i) the at least one polysiloxane in (a) comprises about 15.5% of the composition by weight (w/w %);
(ii) the at least one polysiloxane in (b) comprises about 0.30% of the composition by weight (w/w %);
(iii) the at least one polysiloxane in (c) comprises about 35.5% of the composition by weight (w/w %);
(iv) the at least one formulation-compatible polysiloxane comprises about 37.3% of the composition by weight (w/w %);
(v) the at least one Group X catalyst comprises about 0.0090% (90 ppm) of the composition by weight (w/w %); and
(vi) the at least one additional polysiloxane in (f) comprises about 8.5% of the composition by weight (w/w %).

Embodiment 35 provides the composition of any one of Embodiments 1-30 and 33-34, wherein at least one of the following applies:
(i) the at least one polysiloxane in (a) comprises about 18.90% of the composition by weight (w/w %);
(ii) the at least one polysiloxane in (b) comprises about 0.30% of the composition by weight (w/w %);
(iii) the at least one polysiloxane in (c) comprises about 21.60% of the composition by weight (w/w %);
(iv) the at least one formulation-compatible polysiloxane comprises about 45.1% of the composition by weight (w/w %);
(v) the at least one Group X catalyst comprises about 0.0108% (108 ppm) of the composition by weight (w/w %); and
(vi) the at least one additional polysiloxane in (f) comprises about 10.4% of the composition by weight (w/w %).

Embodiment 36 provides the composition of any one of Embodiments 1-30 and 33-34, wherein at least one of the following applies:
(i) the at least one polysiloxane in (a) comprises about 17.18% of the composition by weight (w/w %);
(ii) the at least one polysiloxane in (b) comprises about 0.26% of the composition by weight (w/w %);
(iii) the at least one polysiloxane in (c) comprises about 19.69% of the composition by weight (w/w %);
(iv) the at least one formulation-compatible polysiloxane comprises about 44.00% of the composition by weight (w/w %);
(v) the at least one Group X catalyst comprises about 0.0099% (99 ppm) of the composition by weight (w/w %); and
(vi) the at least one additional polysiloxane in (f) comprises about 9.45% of the composition by weight (w/w %).

Embodiment 37 provides a method of preparing the composition of Embodiment 1, the method comprising:
(i) contacting each of the following to provide a first mixture:
   at least one polysiloxane in (a) comprising a number of diorganosiloxy monomers, at least one 1-alkenyl-1-organosiloxy monomer, and two termini, wherein each terminus is independently selected from a triorganosiloxy group and a 1,1-organo-1-alkenyl-siloxy group, and wherein at least three Si atoms present in the polysiloxane are alkenyl-substituted;
   at least one polysiloxane in (b) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H); and at least one inert formulation-compatible polysiloxane in (d);
(ii) contacting the first mixture with a Group X transition metal catalyst to provide an at least partially cross-linked mixture; and
(iii) contacting the at least partially cross-linked mixture with:
   at least one polysiloxane in (c) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted.

Embodiment 38 provides a method of preparing the composition of Embodiment 1, the method comprising:
(i) contacting each of the following to provide a first mixture:
   at least one polysiloxane in (a) comprising a number of diorganosiloxy monomers, at least one 1-alkenyl-1-organosiloxy monomer, and two termini, wherein each terminus is independently selected from a triorganosiloxy group and a 1,1-organo-1-alkenyl-siloxy group, and wherein at least three Si atoms present in the polysiloxane are alkenyl-substituted; and
   at least one polysiloxane in (b) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H);
(ii) contacting the first mixture with at least one inert formulation-compatible polysiloxane (d) and a Group X transition metal catalyst to provide an at least partially cross-linked mixture; and
(iii) contacting the at least partially cross-linked mixture with:
   at least one polysiloxane (c) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted.

Embodiment 39 provides a method of preparing the composition of Embodiment 2, the method comprising:
(iv) contacting each of the following to provide a first mixture:
   at least one polysiloxane (a) comprising a number of diorganosiloxy monomers, at least one 1-alkenyl-1-organosiloxy monomer, and two termini, wherein each terminus is independently selected from a triorganosiloxy group and a 1,1-organo-1-alkenyl-siloxy group, and wherein at least three Si atoms present in the polysiloxane are alkenyl-substituted;
   least one polysiloxane (f) comprising a number of diorganosiloxy monomers, optionally a 1,1-diorgano-hydrosiloxy monomer, and two termini, wherein each terminus is independently selected from a triorganosiloxy group and a 1,1-diorgano-hydrosiloxy group, and wherein one Si atom present in the polysiloxane is substituted with H (i.e., Si—H);
   at least one inert formulation-compatible polysiloxane (d); and
   at least one Group X transition metal catalyst;
(v) contacting the first mixture with the following to provide a second mixture:
   at least one polysiloxane (b) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H); and
(vi) contacting the second mixture with:
   least one polysiloxane (c) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted.

Embodiment 40 provides the method of any one of Embodiments 37-39, wherein the at least one polysiloxane (a) possesses at least one of the following properties:
(i) a viscosity ranging from about 800 cSt to about 1200 cSt;
(ii) an average molecular weight ranging from about 15 kDa to about 45 kDa; and
(iii) an alkenyl equivalent per kilogram ranging from about 0.01 to about 0.70.

Embodiment 41 provides the method of any one of Embodiments 37-38, wherein the at least one polysiloxane (b) possesses at least one of the following properties:
(i) a viscosity of about 10,000 cSt;
(ii) an average molecular weight ranging from about 45 kDa to about 75 kDa; and
(iii) a hydride weight percent of about 0.0010% to about 0.010%.

Embodiment 42 provides the method of any one of Embodiments 37-38, wherein the at least one polysiloxane (b) possesses at least one of the following properties:
(i) a viscosity of about 7 cSt to about 10 cSt;
(ii) an average molecular weight ranging from about 1 kDa to about 1.1 kDa; and
(iii) a hydride weight percent of about 0.18% to about 0.20%.

Embodiment 43 provides the method of any one of Embodiments 37-42, wherein the at least one polysiloxane (c) possesses at least one of the following properties:
(i) a viscosity ranging from about 5,000 cSt to about 170,000 cSt; and
(ii) a vinyl equivalent per kilogram ranging from about 0.010 to about 0.040.

Embodiment 44 provides the method of any one of Embodiments 39-43, wherein the at least one polysiloxane (f) possesses at least one of the following properties:
(i) a viscosity ranging from about 150 cSt to about 250 cSt;
(ii) an average molecular weight ranging from about 5 kDa to about 15 kDa; and
(iii) a weight fraction ranging from about 0.01% to about 75% of the reactant composition.

Embodiment 45 provides the method of any one of Embodiments 37-44, wherein the at least one polysiloxane (a) is a compound of formula (Ia):

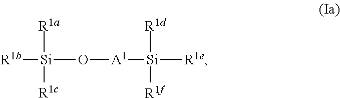

wherein:
A¹ comprises m units of

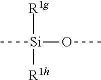

monomer and n units of

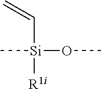

monomer,
   wherein each ---- bond is a Si—O bond;
m is an integer ranging from 410 to 470;
n is an integer ranging from 1 to 50;
R^{1a}, R^{1b}, R^{1c}, R^{1d}, R^{1e}, and R^{1f} are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, optionally substituted C₂-C₁₂ heteroaryl, and C₂-C₆ alkenyl,
   wherein R^{1a}, R^{1b}, R^{1c}, R^{1d}, R^{1e}, and R^{1f} are selected such that each Si atom is substituted with no more than one optionally substituted C₂-C₆ alkenyl; and
R^{1g}, R^{1h}, and R¹ⁱ are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl.

Embodiment 46 provides the method of Embodiment 45, wherein R^{1a}, R^{1b}, R^{1c}, R^{1d}, R^{1e}, R^{1f}, R^{1g}, R^{1h}, R¹ⁱ, m, and n are selected such that the at least one polysiloxane (a) possesses at least one of the following properties:
(i) a viscosity ranging from about 800 cSt to about 1200 cSt;
(ii) an average molecular weight ranging from about 15 kDa to about 45 kDa; and
(iii) an alkenyl equivalent per kilogram ranging from about 0.01 to about 0.70.

Embodiment 47 provides the method of any one of Embodiments 37-46, wherein the at least one polysiloxane (b) is a compound of formula (Ib):

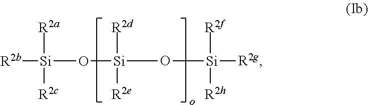

wherein:
R^{2a}, R^{2b}, R^{2c}, R^{2f}, R^{2g}, and R^{2h} are each independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
each occurrence of R^{2d} and R^{2e} is independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
o is an integer ranging from 500 to 1500; and
wherein R^{2a}, R^{2b}, R^{2c}, R^{2d}, R^{2e}, R^{2f}, R^{2g}, and R^{2h} are selected such that each Si atom is substituted with no more than one H atom.

Embodiment 48 provides the method of Embodiment 47, wherein R^{2a}, R^{2b}, R^{2c}, R^{2d}, R^{2e}, R^{2f}, R^{2g}, R^{2h}, and o are selected such that the at least one polysiloxane (b) possesses at least one of the following properties:
(i) a viscosity of about 10,000 cSt;
(ii) an average molecular weight ranging from about 45 kDa to about 75 kDa; and
(iii) a hydride weight percent of about 0.0010% to about 0.010%.

Embodiment 49 provides the method of Embodiment 47, wherein R^{2a}, R^{2b}, R^{2c}, R^{2d}R^{2e}, R^{2f}, R^{2g}, R^{2h}, and o are selected such that the at least one polysiloxane (b) possesses at least one of the following properties:
(i) a viscosity of about 7 cSt to about 10 cSt;
(ii) an average molecular weight ranging from about 1 kDa to about 1.1 kDa; and
(iii) a hydride weight percent of about 0.18% to about 0.20%.

Embodiment 50 provides the method of any one of Embodiments 37-49, wherein the at least one polysiloxane (c) is a compound of formula (Ic):

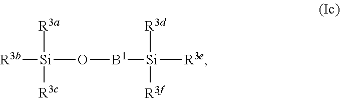

wherein:
B¹ comprises p units of

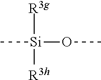

monomer and q units of

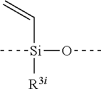

monomer,
   wherein each ---- bond is a Si—O bond;
R^{3a}, R^{3b}, R^{3c}, R^{3d}, R^{3e}, and R^{3f} are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, optionally substituted C₂-C₁₂ heteroaryl, and optionally substituted C₂-C₆ alkenyl,
   wherein R^{3a}, R^{3b}, R^{3c}, R^{3d}, R^{3e}, and R^{3f} are selected such that each Si atom is substituted with no more than one C₂-C₆ alkenyl;
R^{3g}, R^{3h}, and R³ⁱ are each independently C₁-C₆ alkyl;
p is an integer ranging from 500 to 2000; and
q is an integer ranging from 0 to 50.

Embodiment 51 provides the method of Embodiment 50, wherein R^{3a}, R^{3b}, R^{3c}, R^{3d}, R^{3e}, R^{3f}, p, and q, are selected such that the at least one polysiloxane (c) possesses at least one of the following properties:
(i) a viscosity ranging from about 5,000 cSt to about 170,000 cSt; and
(ii) an alkenyl equivalent per kilogram ranging from about 0.010 to about 0.040.

Embodiment 52 provides the method of any one of Embodiments 39-51, wherein the at least one polysiloxane (f) is a compound of formula (If):

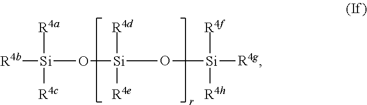

wherein:
R^{4a}, R^{4b}, R^{4c}, R^{4f}, and R^{4g} are each independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
each occurrence of R^{4d} and R^{4e} is independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
r is an integer ranging from 100 to 400; and
wherein no more than one of R^{4a}, R^{4b}, R^{4c}, R^{4d}, R^{4e}, R^{4f}, R^{4g}, and R^{4h} is H.

Embodiment 53 provides the method of Embodiment 52, wherein R^{4a}, R^{4b}, R^{4c}, R^{4d}, R^{4e}, R^{4f}, R^{4g}, R^{4h}, and r are selected such that the at least one polysiloxane (f) possesses at least one of the following properties:
(i) a viscosity ranging from about 150 cSt to about 250 cSt;
(ii) an average molecular weight ranging from about 5 kDa to about 15 kDa; and
(iii) a weight fraction ranging from about 0.01% to about 75% of the reactant composition.

Embodiment 54 provides the method of any one of Embodiments 37-53, wherein the Group X catalyst comprises Pt.

Embodiment 55 provides the method of Embodiment 54, wherein the Pt is Pt(0).

Embodiment 56 provides the method of any one of Embodiments 37-55, wherein the Group X catalyst is Karstedt's catalyst:

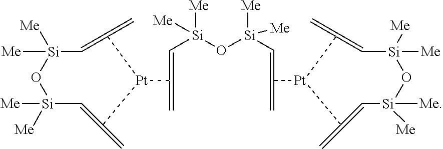

Embodiment 57 provides the method of any one of Embodiments 37-56, wherein the inert formulation-compatible polysiloxane is a compound of formula (Id):

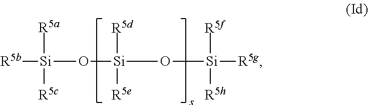

wherein:
R^{5a}, R^{5b}, R^{5c}, R^{5d}, R^{5e}, R^{5f}, R^{5g}, and R^{5h} are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl; and
s is an integer ranging from 1 to about 500.

Embodiment 58 provides the method of any one of Embodiments 37-57, wherein the inert formulation-compatible polysiloxane is selected from the group consisting of poly dimethylsiloxane, dimethiconol, disiloxane, trisiloxane, and diphenyl dimethicone/vinyl diphenyl dimethicone/silsesquioxane cross-polymer.

Embodiment 59 provides the method of any one of Embodiments 37-56 and 58, wherein the inert formulation-compatible polysiloxane is decamethylcyclopentasiloxane.

Embodiment 60 provides the method of any one of Embodiments 44-59, wherein at least one of the following applies:
(a) R^{1a}, R^{1b}, R^{1c}, R^{1d}, R^{1e}, R^{1f}, R^{1g}, R^{1h}, and R¹ⁱ are each independently CH₃ or CH═CH₂;
(b) R^{2a}, R^{2b}, R^{2c}, R^{2d}, R^{2e}, R^{2f}, R^{2g}, and R^{2h} are each independently H or CH₃;
(c) R^{3a}, R^{3b}, R^{3c}, R^{3d}, R^{3e}, and R^{3f} are each independently CH₃ or CH═CH₂;
(d) R^{3g}, R^{3h}, and R³ⁱ are each independently CH₃ or CH═CH₂;
(e) R^{4a}, R^{4b}, R^{4c}, R^{4d}, R^{4e}, R^{4f}, R^{4g}, and R^{4h} are each independently H or CH₃; and
(f) R^{5a}, R^{5b}, R^{5c}, R^{5d}, R^{5e}, R^{5f}, R^{5g}, and R^{5h} are each independently CH₃.

Embodiment 61 provides the method of any one of Embodiments 37-60, wherein the composition further comprises at least one additive.

Embodiment 62 provides the method of Embodiment 61, wherein the additive is at least one selected from the group consisting of glycerin, cetyl diglyceryl tris(trimethylsiloxy)silylethyl dimethicone, hexamethyldisilazane (HMDS) fumed silica, and polyoxyethylene/polyoxypropylene copolymer (PEG/PPG-18/18 dimethicone).

Embodiment 63 provides the method of any one of Embodiments 37-62, wherein the composition has a viscosity ranging from about 4,000 cSt to about 100,000 cSt.

Embodiment 64 provides the method of any one of Embodiments 37-63, wherein one of the following applies:
(a) the composition has a viscosity ranging from about 4,000 cSt to about 8,000 cSt; or
(b) the composition has a viscosity ranging from about 30,000 to about 50,000 cSt.

Embodiment 65 provides the method of any one of Embodiments 37-64, wherein the composition has a total ratio of units of silicon hydride (i.e., Si—H) to vinyl-substituted silicon (i.e., Si—C(H)═CH₂) in all reactant components ranging from about 0.01 to about 0.8.

Embodiment 66 provides the method of any one of Embodiments 37-65, wherein the composition has a total ratio of units of silicon hydride (i.e., Si—H) to vinyl-substituted silicon (i.e., Si—C(H)═CH₂) in all polysiloxane reactant components ranging from about 0.4 to about 0.6.

Embodiment 67 provides the method of any one of Embodiments 39-66, wherein at least one of the following applies:
(a) the at least one polysiloxane (a) comprises trimethylsiloxy terminated, 0.8-1.2% vinylmethylsiloxane dimethylsiloxane copolymer;
(b) the at least one polysiloxane (b) comprises hydride terminated polydimethylsiloxane;
(c) the at least one polysiloxane (c) comprises vinyl terminated polydimethylpolysiloxane;
(d) the at least one inert formulation-compatible polysiloxane comprises polydimethy siloxane and/or decamethylcyclopentasiloxane;
(e) the at least one Group X transition metal catalyst comprises Karstedt's catalyst; and
(f) the at least one polysiloxane (f) comprises monohydride terminated polydimethylpolysiloxane.

Embodiment 68 provides the method of any one of Embodiments 39-67, wherein at least one of the following applies:
(a) the at least one polysiloxane (a) comprises about 6.0% to about 12.0% of the composition by weight (w/w %);
(b) the at least one polysiloxane (b) comprises about 6.0% to about 12.0% of the composition by weight (w/w %);
(c) the at least one polysiloxane (c) comprises about 20.0% to about 30.0% of the composition by weight (w/w %);
(d) the at least one formulation-compatible polysiloxane comprises about 10.0% to about 60.0% of the composition by weight (w/w %);
(e) the at least one Group X catalyst comprises about 0.001% (10 ppm) to about 0.02% (200 ppm) of the composition by weight (w/w %); and
(f) the at least one polysiloxane (f) comprises about 1.0% to about 10.0% of the composition by weight (w/w %).

Embodiment 69 provides the method of any one of Embodiments 39-68, wherein at least one of the following applies:
(a) the at least one polysiloxane (a) comprises about 9.0% of the composition by weight (w/w %);
(b) the at least one polysiloxane (b) comprises about 9.4% of the composition by weight (w/w %);
(c) the at least one polysiloxane (c) comprises about 24.2% of the composition by weight (w/w %);
(d) the at least one formulation-compatible polysiloxane comprises about 49% of the composition by weight (w/w %);
(e) the at least one Group X catalyst comprises about 0.0048% (48 ppm) of the composition by weight (w/w %); and
(f) the at least one additional polysiloxane (f) comprises about 5.8% of the composition by weight (w/w %).

Embodiment 70 provides the method of any one of Embodiments 39-66, wherein at least one of the following applies:
(i) the at least one polysiloxane in (a) comprises about 14.0% to about 20.0% of the composition by weight (w/w %);
(ii) the at least one polysiloxane in (b) comprises about 0.20% to about 0.40% of the composition by weight (w/w %);
(iii) the at least one polysiloxane in (c) comprises about % of the composition by weight (w/w %);
(iv) the at least one formulation-compatible polysiloxane comprises about 35% to about 50% of the composition by weight (w/w %);
(v) the at least one Group X catalyst comprises about 0.0080% (80 ppm) to about 0.0120 (120 ppm) of the composition by weight (w/w %); and
(vi) the at least one additional polysiloxane in (f) comprises about 7% to about 12% of the composition by weight (w/w %).

Embodiment 71 provides the method of any one of Embodiments 39-66 and 70, wherein at least one of the following applies:
(i) the at least one polysiloxane in (a) comprises about 15.5% of the composition by weight (w/w %);
(ii) the at least one polysiloxane in (b) comprises about 0.30% of the composition by weight (w/w %);
(iii) the at least one polysiloxane in (c) comprises about 35.5% of the composition by weight (w/w %);
(iv) the at least one formulation-compatible polysiloxane comprises about 37.3% of the composition by weight (w/w %);
(v) the at least one Group X catalyst comprises about 0.0090% (90 ppm) of the composition by weight (w/w %); and
(vi) the at least one additional polysiloxane in (f) comprises about 8.5% of the composition by weight (w/w %)

Embodiment 72 provides the method of any one of Embodiments 39-66 and 70-71, wherein at least one of the following applies:
(i) the at least one polysiloxane in (a) comprises about 18.90% of the composition by weight (w/w %);
(ii) the at least one polysiloxane in (b) comprises about 0.30% of the composition by weight (w/w %);
(iii) the at least one polysiloxane in (c) comprises about 21.60% of the composition by weight (w/w %);
(iv) the at least one formulation-compatible polysiloxane comprises about 45.1% of the composition by weight (w/w %);
(v) the at least one Group X catalyst comprises about 0.0108% (108 ppm) of the composition by weight (w/w %); and
(vi) the at least one additional polysiloxane in (f) comprises about 10.4% of the composition by weight (w/w %).

Embodiment 73 provides a mechanically reinforcing composition (MRC) comprising:
(i) at least one polysiloxane (g) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted;
(ii) at least one polysiloxane (h) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H);
(iii) at least one reinforcing material; and
(iv) at least one silicone miscible, volatile fluid.

Embodiment 74 provides the composition of Embodiment 73, wherein the composition further comprises at least one non-volatile, silicone miscible fluid.

Embodiment 75 provides the composition of Embodiment 73 or 74, wherein the at least one polysiloxane (g) is a compound of formula (Ig):

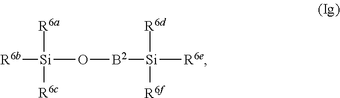

wherein:
B² comprises t units of

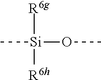

monomer and u units of

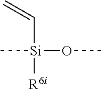

monomer,
   wherein each ---- bond is a Si—O bond;
R^{6a}, R^{6b}, R^{6c}, R^{6d}, R^{6e}, and R^{6f} are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, optionally substituted C₂-C₁₂ heteroaryl, and optionally substituted C₂-C₆ alkenyl,
   wherein R^{6a}, R^{6b}, R^{6c}, R^{6d}, R^{6e}, and R^{6f} are selected such that each Si atom is substituted with no more than one optionally substituted C₂-C₆ alkenyl;
R^{6g}, R^{6h}, and R⁶ⁱ are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
t is an integer ranging from 300 to 2000; and
u is an integer ranging from 0 to 50.

Embodiment 76 provides the composition of any one of Embodiments 73-75, wherein the at least one polysiloxane (h) is a compound of formula (Ih):

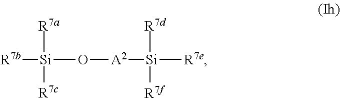

wherein:
A² comprises v units of

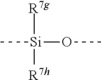

monomer and w units of

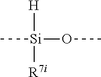

monomer,
   wherein each ---- bond is a Si—O bond;
R^{7a}, R^{7b}, R^{7c}, R^{7d}, R^{7e}, and R^{7f} are each independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl,
   wherein R^{7a}, R^{7b}, R^{7c}, R^{7d}, R^{7e}, and R^{7f} are selected such that each Si atom is substituted with no more than one H atom;
each occurrence of R^{7g}, R^{7h}, and R⁷ⁱ is independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
v is an integer ranging from 10 to 500; and
w is an integer ranging from 2 to 10.

Embodiment 77 provides the composition of any one of Embodiments 73-76, wherein at least one of the following applies:
(a) R^{6a}, R^{6b}, R^{6c}, R^{6d}, R^{6e} and R^{6f} are each independently selected from the group consisting of CH₃ and —CH═CH₂;
(b) R^{6g}, R^{6h}, and R⁶ⁱ are each independently CH₃;
(c) R^{7a}, R^{7b}, R^{7c}, R^{7d}, R^{7e}, and R^{7f} are each independently selected from the group H and CH₃ R^{6g}, R^{6h}, and R⁶ⁱ are each independently CH₃; and
(c) R^{7g}, R^{7h}, and R⁷ⁱ are each independently CH₃.

Embodiment 78 provides the composition of any one of Embodiments 73-77, wherein the silicone miscible, volatile fluid is at least one selected from the group consisting of disiloxane, trisiloxane, and decamethyl cyclopentasiloxane.

Embodiment 79 provides the composition of any one of Embodiments 73-78, wherein the reinforcing agent is at least one selected from the group consisting of silica and HMDS treated fumed silica.

Embodiment 80 provides the composition of any one of Embodiments 73-79, wherein the composition has a total ratio of units of silicon hydride (i.e., Si—H) to vinyl-substituted silicon (i.e., Si—C(H)═CH₂) in all reactant components ranging from about 1 to about 20.

Embodiment 81 provides the composition of any one of Embodiments 73-80, wherein the composition has a total ratio of units of silicon hydride (i.e., Si—H) to vinyl-substituted silicon (i.e., Si—C(H)═CH₂) in all reactant components ranging from about 5 to about 7.

Embodiment 82 provides the composition of any one of Embodiments 73-81, wherein the composition further comprises one or more additives.

Embodiment 83 provides the composition of Embodiment 82, wherein the additive is a rheology modifier.

Embodiment 84 provides the composition of Embodiment 82 or 83, wherein the additive is an aesthetic and/or cosmetic modifier.

Embodiment 85 provides the composition of Embodiment 84, wherein the aesthetic and/or cosmetic modifier is at least one selected from the group consisting of vitamin A, vitamin B3, vitamin C, vitamin D, vitamin E, vitamin F, vitamin K, glycolic acid, sunscreen, and/or panthenol.

Embodiment 86 provides the composition of any one of Embodiments 82-85, wherein the additive is a pharmaceutically active compound and/or composition.

Embodiment 87 provides the composition of Embodiment 86, wherein the pharmaceutically active additive is at least one selected from the group consisting of one or more steroids (e.g., mometasone, clobetasol, triamcinolone, fluocinonide, flurandrenolide, clocortolone, halobetasol, desoximetasone, desonide, hydrocortisone, betamethasone, fluticasone, halcinonide, fluocinolone, prednicarbate, diflorasone, flurandrenolide, amcinonide and alclometasone), one or more retinoids (e.g., tretinoin, adapalene, tazarotene, alitretinoin and bexarotene), benzoyl peroxide, azelaic acid, diamino-diphenyl sulphone, one or more JAK inhibitors (e.g., ruxolitinib and delgocitinib), one or more antibiotics (e.g., fusidic acid, mupirocin, retapamulin, silver sulfadiazine, bacitracin, neomycin, polymyxin B, sulfacetamide sodium, sulfur, ozenoxacin, silver sulfadiazine, erythromycin, mafenide, gentamicin, clindamycin, metronidazole, gentamicin, and nadifloxacin), one or more calcineurin inhibitors (e.g., tacrolimus and pimecrolimus), one or more antifungals (e.g., clotrimazole, terbinafine, miconazole, econazole, ketoconazole, tioconazole and amorolfine), becaplermin, 5-fluorouracil, diclofenac, and imiquimod.

Embodiment 88 provides the composition of any one of Embodiments 78-87, wherein at least one of the following applies:
(a) the at least one polysiloxane (g) comprises vinyl terminated dimethylpolysiloxane;
(b) the at least one polysiloxane (h) comprises trimethylsiloxy terminated, pendant silicon-hydride functional poly dimethylsiloxane;
(c) the at least one reinforcing material comprises HMDS treated fumed silica; and
(d) the at least one silicone miscible, volatile fluid comprises decamethyl cyclopentasiloxane.

Embodiment 89 provides the composition of Embodiment 88, wherein at least one of the following applies:
(a) the at least one polysiloxane (g) comprises about 30% to about 50% of the composition by weight (w/w %);
(b) the at least one polysiloxane (h) comprises about 1% to about 10% of the composition by weight (w/w %);
(c) the at least one reinforcing material comprises about 10% to about 30% of the composition by weight (w/w %); and
(d) the at least one silicone miscible, volatile fluid comprises about 35% to about 50% of the composition by weight (w/w %).

Embodiment 90 provides the composition of Embodiment 88 or 89, wherein at least one of the following applies:
(a) the at least one polysiloxane (g) comprises about 36.6% of the composition by weight (w/w %);
(b) the at least one polysiloxane (h) comprises about 6.2% of the composition by weight (w/w %);
(c) the at least one reinforcing material comprises about 14.5% of the composition by weight (w/w %); and
(d) the at least one silicone miscible, volatile fluid comprises about 42.7% of the composition by weight (w/w %).

Embodiment 91 provides the composition of any one of Embodiments 73-87, wherein at least one of the following applies:
(a) the at least one polysiloxane (g) comprises vinyl terminated dimethylpolysiloxane;
(b) the at least one polysiloxane (h) comprises trimethylsiloxy terminated, pendant silicon-hydride functional poly dimethylsiloxane;
(c) the at least one reinforcing material comprises silica and HMDS treated fumed silica;
(d) the at least one silicone miscible, volatile fluid comprises decamethyl cyclopentasiloxane; and
(e) the at least one non-volatile silicone miscible fluid comprises polydimethylsiloxane fluid.

Embodiment 92 provides the composition of any one of Embodiments 73-87 and 91, wherein at least one of the following applies:
(a) the at least one polysiloxane (g) comprises about 20% to about 40% of the composition by weight (w/w %);
(b) the at least one polysiloxane (h) comprises about 1% to about 10% of the composition by weight (w/w %);
(c) the at least one reinforcing material comprises about 10% to about 30% of the composition by weight (w/w %);
(d) the at least one silicone miscible, volatile fluid comprises about 35% to about 50% of the composition by weight (w/w %); and
(e) the at least one non-volatile silicone miscible fluid comprises about 0.1 to about 5% of the composition by weight (w/w %).

Embodiment 93 provides the composition of Embodiment 92, wherein at least one of the following applies:
(a) the at least one polysiloxane (g) comprises about 34.4%, 34.8%, or about 36.2% of the composition by weight (w/w %);
(b) the at least one polysiloxane (h) comprises about 5.8%, 5.9%, or about 6.1% of the composition by weight (w/w %);
(c) the at least one reinforcing material comprises two components which in total comprise about 14.4%, 18.6%, or about 18.8% of the composition by weight (w/w %);
(d) the at least one silicone miscible, volatile fluid comprises about 40.1%, 40.6%, or about 42.3% of the composition by weight (w/w %); and
(e) the at least one non-volatile silicone miscible fluid comprises about 0% or about 1% of the composition by weight (w/w %).

Embodiment 94 provides the composition of any one of Embodiments 73-87 and 91-93, wherein at least one of the following applies:
(a) the at least one polysiloxane (g) comprises about 30% of the composition by weight (w/w %);
(b) the at least one polysiloxane (h) comprises about 5% of the composition by weight (w/w %);
(c) the at least one reinforcing material comprises about 22% of the composition by weight (w/w %);
(d) the at least one silicone miscible, volatile fluid comprises about 42% of the composition by weight (w/w %); and
(e) the at least one non-volatile silicone miscible fluid comprises about 0.7% of the composition by weight (w/w %).

Embodiment 95 provides the composition of any one of Embodiments 73-87 and 91-94, wherein at least one of the following applies:
(a) the at least one polysiloxane (g) comprises two vinyl terminated dimethylpolysiloxanes;
(b) the at least one polysiloxane (h) comprises two trimethylsiloxy terminated, pendant silicon-hydride functional polydimethylsiloxanes;
(c) the at least one reinforcing material comprises silica and HMDS treated fumed silica; and
(d) the at least one silicone miscible, volatile fluid comprises decamethyl cyclopentasiloxane.

Embodiment 96 provides the composition of any one of Embodiments 73-87 and 95, wherein at least one of the following applies:
(a) the at least one polysiloxane (g) comprises about 20% to about 40% of the composition by weight (w/w %);
(b) the at least one polysiloxane (h) comprises about 1% to about 10% of the composition by weight (w/w %);
(c) the at least one reinforcing material comprises about 5% to about 25% of the composition by weight (w/w %); and
(d) the at least one silicone miscible, volatile fluid comprises about 40% to about 60% of the composition by weight (w/w %).

Embodiment 97 provides the composition of any one of Embodiments 73-87 and 95-96, wherein at least one of the following applies:
(a) the at least one polysiloxane (g) comprises about 27% of the composition by weight (w/w %);
(b) the at least one polysiloxane (h) comprises about 6% of the composition by weight (w/w %);
(c) the at least one reinforcing material comprises about 13% of the composition by weight (w/w %); and
(d) the at least one silicone miscible, volatile fluid comprises about 54% of the composition by weight (w/w %).

Embodiment 98 provides a multilayer composition comprising:
(a) an adhesive basal layer comprising the composition of any one of Embodiments 1-36, wherein the adhesive basal layer optionally further comprises one or more additional compounds and/or compositions selected from the group consisting of a non-reactive fluid, emulsifier, a cosmetic, and a pharmaceutically active agent and/or composition;
(b) a mechanically reinforcing layer comprising the composition of any one of Embodiments 73-97;
   wherein the adhesive basal layer is in contiguous contact with at least a portion of a surface of an object; and
   wherein the mechanically reinforcing layer is in contiguous contact with at least a portion of the surface of the adhesive basal layer.

Embodiment 99 provides a method for applying a multilayered wound dressing composition to a wound of a subject, the method comprising:
(a) applying to the surface of the wound an adhesive basal layer comprising the composition of any one of Embodiments 1-36, wherein the adhesive basal layer optionally further comprises one or more additional compounds and/or compositions selected from the group consisting of a non-reactive fluid, emulsifier, and a pharmaceutically active agent and/or composition; and
(b) applying to the surface of the adhesive basal layer a mechanically reinforcing layer comprising the composition of any one of Embodiments 73-97;
   wherein the adhesive basal layer is in contiguous contact with at least a portion of the surface of the wound; and
   wherein the mechanically reinforcing layer is in contiguous contact with at least a portion of the surface of the adhesive basal layer.

Embodiment 100 provides the method of Embodiment 99, wherein the adhesive basal layer is applied with a roller.

Embodiment 101 provides the method of Embodiment 100, wherein the roller is metal.

Embodiment 102 provides the method of any one of Embodiments 99-101, wherein the adhesive basal layer has a thickness of about 10 μm to about 100 μm.

Embodiment 103 provides the method of any one of Embodiments 99-102, wherein the mechanically reinforcing layer is applied with an applicator tool, wherein the applicator is optionally metal, and wherein the applicator is optionally a roller.

Embodiment 104 provides the method of any one of Embodiments 99-103, wherein at least one of the adhesive basal layer and the mechanically reinforcing layer have a uniform thickness.

Embodiment 105 provides the method of any one of Embodiments 99-104, wherein the wound is caused by mechanical shearing and/or puncturing of the skin of the subject.

Embodiment 106 provides the method of any one of Embodiments 99-105, wherein the wound is caused by a skin condition.

Embodiment 107 provides the method of Embodiment 106, wherein the skin condition is at least one of xerosis, ichthyosis, eczema, contact dermatitis, diaper rash, radiation dermatitis, and psoriasis.

Embodiment 108 provides the method of any one of Embodiments 99-107, wherein the adhesive basal layer is applied to a wound which has been treated and/or coated with one or more topically active compounds and/or compositions.

Embodiment 109 provides a method of treating a skin condition and/or wound of a subject, the method comprising:
(a) applying to the surface of the wound an adhesive basal layer comprising the composition of any one of Embodiments 1-36, wherein the adhesive basal layer optionally further comprises one or more additional compounds and/or compositions selected from the group consisting of a non-reactive fluid, emulsifier, a cosmetic, and a pharmaceutically active agent and/or composition; and
(b) applying to the surface of the adhesive basal layer a mechanically reinforcing layer comprising the composition of any one of Embodiments 73-97;
   wherein the adhesive basal layer is in contiguous contact with at least a portion of the surface of the wound; and
   wherein the mechanically reinforcing layer is in contiguous contact with at least a portion of the surface of the adhesive basal layer.

Embodiment 110 provides the method of Embodiment 109, wherein the adhesive basal layer is applied with a roller.

Embodiment 111 provides the method of Embodiment 110, wherein the roller is metal.

Embodiment 112 provides the method of any one of Embodiments 109-111, wherein the adhesive basal layer has a thickness of about 10 μm to about 100 μm.

Embodiment 113 provides the method of any one of Embodiments 109-112, wherein the mechanically reinforcing layer has a thickness of about 10 μm to about 100 μm.

Embodiment 114 provides the method of any one of Embodiments 109-113, wherein the mechanically reinforcing layer is applied with an applicator tool, wherein the tool is optionally metal, and wherein the applicator is optionally a roller.

Embodiment 115 provides the method of any one of Embodiments 109-114, wherein at least one of the adhesive basal layer and the mechanically reinforcing layer have a uniform thickness.

Embodiment 116 provides the method of any one of Embodiments 109-115, wherein the wound is caused by mechanical shearing and/or puncturing of the skin of the subject.

Embodiment 117 provides the method of any one of Embodiments 109-116, wherein the wound is caused by a skin condition.

Embodiment 118 provides the method of Embodiment 117, wherein the skin condition is selected from the group consisting of xerosis, ichthyosis, eczema, contact dermatitis, diaper rash, radiation dermatitis, and psoriasis.

Embodiment 119 provides the method of any one of Embodiments 109-118, wherein the adhesive basal layer is applied to a wound which has been treated and/or coated with one or more topically active compounds and/or compositions.

Embodiment 120 provides a kit comprising:
(a) a container comprising the composition of any one of Embodiments 1-36, wherein the container is suitable for dispensation;
(b) a container comprising the composition of any one of Embodiments 73-97, wherein the container is suitable for dispensation; and
(c) instructional materials for use thereof.

Embodiment 121 provides the kit of Embodiment 120, further comprising a roller.

Embodiment 122 provides the kit of Embodiment 121, wherein the roller is metal.

Embodiment 123 provides the kit of any one of Embodiments 120-122, further comprising an applicator.

Embodiment 124 provides the kit of Embodiment 123, wherein the applicator is metal.

Embodiment 125 provides the kit of Embodiment 123 or 124, wherein the applicator is a roller.

Embodiment 126 provides the kit of any one of Embodiments 123-125, wherein the kit further comprises applicator maintenance wipes.

Embodiment 127 provides an emulsion composition comprising:
(a) at least one cross-linked telechelic polymer composition,
   wherein the at least one cross-linked telechelic polymer composition comprises about 1% to about 25% of the emulsion composition (w/w %);
(b) at least one emulsifier,
   wherein the at least one emulsifier comprises about 0.1% to about 10% of the emulsion composition (w/w %);
(c) at least one polar solvent or water-miscible solvent,
   wherein the at least one polar solvent or water-miscible solvent comprises about 50% to about 99% of the emulsion composition (w/w %); and
(d) at least one silicone fluid,
   wherein the at least one silicone fluid comprises about 1% to about 25% of the emulsion composition (w/w %).

Embodiment 128 provides the emulsion of Embodiment 127, wherein at least one of the following applies:
(a) the at least one cross-linked telechelic polymer composition is the composition of any one of Embodiments 1-36;
(b) the at least one cross-linked telechelic polymer composition comprises about 10% of the emulsion composition (w/w %);
(c) the at least one emulsifier comprises cetyl diglyceryl tris(trimethylsiloxy)silylethyl dimethicone;
(d) the at least one emulsifier comprises about 1% of the emulsion composition (w/w %);
(e) the at least one polar solvent or water-miscible solvent comprises at least one selected from the group consisting of 1,3-butylene glycol and glycerin;
(f) the at least one polar solvent or water-miscible solvent comprises about 75% of the emulsion composition (w/w %);
(g) the at least one silicone fluid comprises at least one selected from the group consisting of caprylyl methicone and dimethicone; and
(h) the at least one silicone fluid comprises about 14% of the emulsion composition (v/w %).

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A telechelic polymer composition comprising a cross-linked reaction product of any of:
(i) at least one polysiloxane (a) comprising a number of diorganosiloxy monomers, at least one 1-alkenyl-1-organosiloxy monomer, and two termini, wherein each terminus is independently selected from a triorganosiloxy group and a 1,1-organo-1-alkenyl-siloxy group, and wherein at least three Si atoms present in the polysiloxane are alkenyl-substituted;
(ii) at least one polysiloxane (b) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H);
(iii) at least one polysiloxane (c) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted;
(iv) at least one inert formulation-compatible polysiloxane (d); and
(v) at least one Group X transition metal catalyst.

2. The composition of claim 1, further comprising:
(vi) at least one additional polysiloxane (f), wherein the at least one additional polysiloxane comprises a number of diorganosiloxy monomers, optionally a 1,1-diorgano-hydrosiloxy monomer, and two termini, wherein each terminus is independently selected from a triorganosiloxy group and a 1,1-diorgano-hydrosiloxy group, and wherein one Si atom present in the polysiloxane is substituted with H (i.e., Si—H).

3. The composition of claim 1 or 2, wherein the at least one polysiloxane (a) is a compound of formula (Ia): 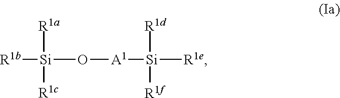
wherein:
A¹ comprises m units of 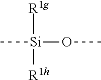
monomer and n units of 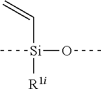
monomer,
wherein each ---- bond is a Si—O bond;
m is an integer ranging from 410 to 470;
n is an integer ranging from 1 to 50;
R^{1a}, R^{1b}, R^{1c}, R^{1d}, R^{1e}, and R^{1f} are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, optionally substituted C₂-C₁₂ heteroaryl, and optionally substituted C₂-C₆ alkenyl,
wherein R^{1a}, R^{1b}, R^{1c}, R^{1d}, R^{1e}, and R^{1f} are selected such that each Si atom is substituted with no more than one optionally substituted C₂-C₆ alkenyl; and
R^{1g}, R^{1h}, and R¹ⁱ are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl.

4. The composition of any one of claims 1-3, wherein the at least one polysiloxane in (b) is a compound of formula (Ib): 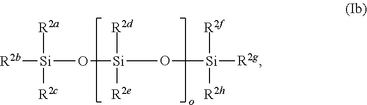
wherein:
R^{2a}, R^{2b}, R^{2c}, R^{2f}, R^{2g}, and R^{2h} are each independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
each occurrence of R^{2d} and R^{2e} is independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
o is an integer ranging from 500 to 1500; and
wherein R^{2a}, R^{2b}, R^{2c}, R^{2d}, R^{2e}, R^{2f}, R^{2g} and R^{2h} are selected such that each Si atom is substituted with no more than one H atom.

5. The composition of any one of claims 1-4, wherein the at least one polysiloxane in (c) is a compound of formula (Ic): 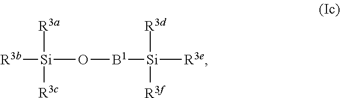
wherein:
B¹ comprises p units of 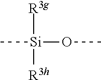
monomer and q units of 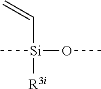
monomer,
wherein each ---- bond is a Si—O bond;
R^{3a}, R^{3b}, R^{3c}, R^{3d}, R^{3e}, and R^{3f} are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-CLO aryl, optionally substituted C₂-C₁₂ heteroaryl, and optionally substituted C₂-C₆ alkenyl,
wherein R^{3a}, R^{3b}, R^{3c}, R^{3d}, R^{3e}, and R^{3f} are selected such that each Si atom is substituted with no more than one C₂-C₆ alkenyl;
R^{3g}, R^{3h}, and R³ⁱ are each independently C₁-C₆ alkyl;
p is an integer ranging from 500 to 2000; and
q is an integer ranging from 0 to 50.

6. The composition of any one of claims 2-5, wherein the at least one polysiloxane in (f) is a compound of formula (If): 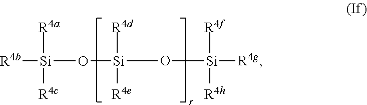
wherein:
R^{4a}, R^{4b}, R^{4c}, R^{4f}, and R^{4g} are each independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
each occurrence of R^{4d} and R^{4e} is independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
r is an integer ranging from 100 to 400; and
wherein no more than one of R^{4a}, R^{4b}, R^{4c}, R^{4d}, R^{4c}, R^{4f}, R^{4g}, and R^{4h} is H.

7. The composition of any one of claims 1-6, wherein the Group X catalyst comprises Pt.

8. The composition of claim 7, wherein the Pt is Pt(0).

9. The composition of any one of claims 1-8, wherein the Group X catalyst is Karstedt's catalyst:

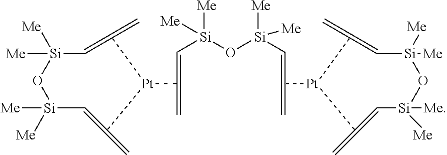

10. The composition of any one of claims 1-9, wherein the inert formulation-compatible polysiloxane is a compound of formula (Id): 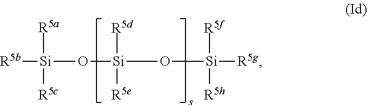
wherein:
R^{5a}, R^{5b}, R^{5c}, R^{5d}, R^{5e}, R^{5f}, R^{5g}, and R^{5h} are each independently C₁-C₆ alkyl; and
s is an integer ranging from 1 to about 500.

11. The composition of any one of claims 1-10, wherein the inert formulation-compatible polysiloxane is selected from the group consisting of poly dimethylsiloxane, dimethiconol, disiloxane, trisiloxane, and diphenyl dimethicone/vinyl diphenyl dimethicone/silsesquioxane cross-polymer.

12. The composition of any one of claims 1-9 and 11, wherein the inert formulation-compatible polysiloxane is decamethylcyclopentasiloxane.

13. The composition of any one of claims 1-12, wherein at least one of the following applies:
(i) R^{1a}, R^{1b}, R^{1c}, R^{1d}, R^{1c}, R^{1f}, R^{1g}, R^{1h}, and R¹ⁱ are each independently CH₃ or CH═CH₂;
(ii) R^{2a}, R^{2b}, R^{2c}, R^{2d}, R^{2e}, R^{2f}, R^{2g}, and R^{2h} are each independently H or CH₃;
(iii) R^{3a}, R^{3b}, R^{3c}, R^{3d}, R^{3e}, and R^{3f} are each independently CH₃ or CH═CH₂;
(iv) R^{3g}, R^{3h}, and R³ⁱ are each independently CH₃ or CH═CH₂;
(v) R^{4a}, R^{4b}, R^{4c}, R^{4d}, R^{4e}, R^{4f}, R^{4g}, and R^{4h} are each independently H or CH₃; and
(vi) R^{5a}, R^{5b}, R^{5c}, R^{5d}, R^{5e}, R^{5f}, R^{5g}, and R^{5h} are each independently CH₃.

14. The composition of any one of claims 1-13, wherein the composition further comprises at least one additive.

15. The composition of claim 14, wherein the at least one additive is at least one selected from the group consisting of glycerin, cetyl diglyceryl tris(trimethylsiloxy)silylethyl dimethicone, hexamethyldisilazane (HMDS) fumed silica, and polyoxyethylene/polyoxypropylene copolymer (PEG/PPG-18/18 dimethicone).

16. The composition of any one of claims 1-15, wherein the composition has a viscosity ranging from about 4,000 cSt to about 100,000 cSt.

17. The composition of any one of claims 1-16, wherein one of the following applies:
(a) the composition has a viscosity ranging from about 4,000 cSt to about 8,000 cSt; or
(b) the composition has a viscosity ranging from about 30,000 to about 50,000 cSt.

18. The composition of any one of claims 1-17, wherein the composition has a total ratio of units of silicon hydride (i.e., Si—H) to vinyl-substituted silicon (i.e., Si—C(H)═CH₂) in all reactant components ranging from about 0.01 to about 0.8.

19. The composition of any one of claims 1-18, wherein the composition has a total ratio of units of silicon hydride (i.e., Si—H) to vinyl-substituted silicon (i.e., Si—C(H)═CH₂) in all polysiloxane reactant components ranging from about 0.4 to about 0.6.

20. The composition of any one of claims 1-19, wherein at least one of the following applies:
(i) the at least one polysiloxane in (a) comprises trimethylsiloxy terminated, 0.8-1.2% vinylmethylsiloxane dimethylsiloxane copolymer;
(ii) the at least one polysiloxane in (b) comprises hydride terminated polydimethylsiloxane;
(iii) the at least one polysiloxane in (c) comprises vinyl terminated polydimethylpolysiloxane;
(iv) the at least one inert formulation-compatible polysiloxane comprises polydimethyl siloxane and/or decamethylcyclopentasiloxane;
(v) the at least one Group X transition metal catalyst comprises Karstedt's catalyst; and
(vi) the at least one additional polysiloxane in (f) comprises monohydride terminated polydimethylpolysiloxane.

21. The composition of any one of claims 1-20, wherein at least one of the following applies:
(vii) the at least one polysiloxane in (a) comprises about 6.0% to about 12.0% of the composition by weight (w/w %);
(viii) the at least one polysiloxane in (b) comprises about 6.0% to about 12.0% of the composition by weight (w/w %);
(ix) the at least one polysiloxane in (c) comprises about 20.0% to about 30.0% of the composition by weight (w/w %);
(x) the at least one formulation-compatible polysiloxane comprises about 10.0% to about 60.0% of the composition by weight (w/w %);
(xi) the at least one Group X catalyst comprises about 0.001% (10 ppm) to about 0.02% (200 ppm) of the composition by weight (w/w %); and
(xii) the at least one additional polysiloxane in (f) comprises about 1.0% to about 10.0% of the composition by weight (w/w %).

22. The composition of any one of claims 1-21, wherein at least one of the following applies:
(vii) the at least one polysiloxane in (a) comprises about 9.0% of the composition by weight (w/w %);
(viii) the at least one polysiloxane in (b) comprises about 9.4% of the composition by weight (w/w %);
(ix) the at least one polysiloxane in (c) comprises about 24.2% of the composition by weight (w/w %);
(x) the at least one formulation-compatible polysiloxane comprises about 49% of the composition by weight (w/w %);
(xi) the at least one Group X catalyst comprises about 0.0048% (48 ppm) of the composition by weight (w/w %); and
(xii) the at least one additional polysiloxane in (f) comprises about 5.8% of the composition by weight (w/w %).

23. The composition of any one of claims 1-20, wherein at least one of the following applies:
(i) the at least one polysiloxane in (a) comprises about 14.0% to about 20.0% of the composition by weight (w/w %);
(ii) the at least one polysiloxane in (b) comprises about 0.20% to about 0.40% of the composition by weight (w/w %);
(iii) the at least one polysiloxane in (c) comprises about 18% to about 40% of the composition by weight (w/w %);
(iv) the at least one formulation-compatible polysiloxane comprises about 35% to about 50% of the composition by weight (w/w %);
(v) the at least one Group X catalyst comprises about 0.0080% (80 ppm) to about 0.0120 (120 ppm) of the composition by weight (w/w %); and
(vi) the at least one additional polysiloxane in (f) comprises about 7% to about 12% of the composition by weight (w/w %).

24. The composition of any one of claims 1-20 and 23, wherein at least one of the following applies:
(i) the at least one polysiloxane in (a) comprises about 15.5% of the composition by weight (w/w %);
(ii) the at least one polysiloxane in (b) comprises about 0.30% of the composition by weight (w/w %);
(iii) the at least one polysiloxane in (c) comprises about 35.5% of the composition by weight (w/w %);
(iv) the at least one formulation-compatible polysiloxane comprises about 37.3% of the composition by weight (w/w %);
(v) the at least one Group X catalyst comprises about 0.0090% (90 ppm) of the composition by weight (w/w %); and
(vi) the at least one additional polysiloxane in (f) comprises about 8.5% of the composition by weight (w/w %).

25. The composition of any one of claims 1-20 and 23-24, wherein at least one of the following applies:
(i) the at least one polysiloxane in (a) comprises about 18.90% of the composition by weight (w/w %);
(ii) the at least one polysiloxane in (b) comprises about 0.30% of the composition by weight (w/w %);
(iii) the at least one polysiloxane in (c) comprises about 21.60% of the composition by weight (w/w %);
(iv) the at least one formulation-compatible polysiloxane comprises about 45.1% of the composition by weight (w/w %);
(v) the at least one Group X catalyst comprises about 0.0108% (108 ppm) of the composition by weight (w/w %); and
(vi) the at least one additional polysiloxane in (f) comprises about 10.4% of the composition by weight (w/w %).

26. The composition of any one of claims 1-20 and 23-24, wherein at least one of the following applies:
(i) the at least one polysiloxane in (a) comprises about 17.18% of the composition by weight (w/w %);
(ii) the at least one polysiloxane in (b) comprises about 0.26% of the composition by weight (w/w %);
(iii) the at least one polysiloxane in (c) comprises about 19.69% of the composition by weight (w/w %);
(iv) the at least one formulation-compatible polysiloxane comprises about 44.00% of the composition by weight (w/w %);
(v) the at least one Group X catalyst comprises about 0.0099% (99 ppm) of the composition by weight (w/w %); and
(vi) the at least one additional polysiloxane in (f) comprises about 9.45% of the composition by weight (w/w %).

27. A method of preparing the composition of claim 1, the method comprising:
(i) contacting each of the following to provide a first mixture:
at least one polysiloxane in (a) comprising a number of diorganosiloxy monomers, at least one 1-alkenyl-1-organosiloxy monomer, and two termini, wherein each terminus is independently selected from a triorganosiloxy group and a 1,1-organo-1-alkenyl-siloxy group, and wherein at least three Si atoms present in the polysiloxane are alkenyl-substituted;
at least one polysiloxane in (b) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H); and
at least one inert formulation-compatible polysiloxane in (d);
(ii) contacting the first mixture with a Group X transition metal catalyst to provide an at least partially cross-linked mixture; and
(iii) contacting the at least partially cross-linked mixture with:
at least one polysiloxane in (c) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted.

28. A method of preparing the composition of claim 1, the method comprising:
(i) contacting each of the following to provide a first mixture:
at least one polysiloxane in (a) comprising a number of diorganosiloxy monomers, at least one 1-alkenyl-1-organosiloxy monomer, and two termini, wherein each terminus is independently selected from a triorganosiloxy group and a 1,1-organo-1-alkenyl-siloxy group, and wherein at least three Si atoms present in the polysiloxane are alkenyl-substituted; and
at least one polysiloxane in (b) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H);
(ii) contacting the first mixture with at least one inert formulation-compatible polysiloxane (d) and a Group X transition metal catalyst to provide an at least partially cross-linked mixture; and
(iii) contacting the at least partially cross-linked mixture with:
at least one polysiloxane (c) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted.

29. A method of preparing the composition of claim 2, the method comprising:
(vii) contacting each of the following to provide a first mixture:
at least one polysiloxane (a) comprising a number of diorganosiloxy monomers, at least one 1-alkenyl-1-organosiloxy monomer, and two termini, wherein each terminus is independently selected from a triorganosiloxy group and a 1,1-organo-1-alkenyl-siloxy group, and wherein at least three Si atoms present in the polysiloxane are alkenyl-substituted;
least one polysiloxane (f) comprising a number of diorganosiloxy monomers, optionally a 1,1-diorgano-hydrosiloxy monomer, and two termini, wherein each terminus is independently selected from a triorganosiloxy group and a 1,1-diorgano-hydrosiloxy group, and wherein one Si atom present in the polysiloxane is substituted with H (i.e., Si—H);
at least one inert formulation-compatible polysiloxane (d); and
at least one Group X transition metal catalyst;
(viii) contacting the first mixture with the following to provide a second mixture:
at least one polysiloxane (b) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H); and
(ix) contacting the second mixture with:
least one polysiloxane (c) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted.

30. A mechanically reinforcing composition (MRC) comprising:
(i) at least one polysiloxane (g) comprising a number of diorganosiloxy monomers, optionally a number of 1-alkenyl-1-organo-siloxy monomers, and two termini, wherein each terminus is independently selected from a triorganosiloxy group and a 1,1-diorgano-1-alkenyl-siloxy group, and wherein at least two Si atoms present in the polysiloxane are vinyl-substituted;
(ii) at least one polysiloxane (h) comprising a number of diorganosiloxy monomers, a number of 1-organo-hydrosiloxy monomers, and two termini, wherein each terminus is independently selected from a triorganosiloxy group and a 1,1-diorgano-hydrosiloxy group, and wherein at least two Si atoms present in the polysiloxane are substituted with H (i.e., Si—H);
(iii) at least one reinforcing material; and
(iv) at least one silicone miscible, volatile fluid.

31. The MRC of claim 30, wherein the composition further comprises at least one non-volatile, silicone miscible fluid.

32. The MRC of claim 30 or 31, wherein the at least one polysiloxane (g) is a compound of formula (Ig): 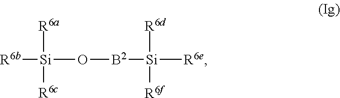
wherein:
B² comprises t units of 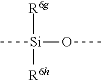
monomer and u units of 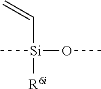
monomer,
wherein each ---- bond is a Si—O bond;
R^{6a}, R^{6b}, R^{6c}, R^{6d}, R^{6e}, and R^{6f} are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, optionally substituted C₂-C₁₂ heteroaryl, and optionally substituted C₂-C₆ alkenyl,
wherein R^{6a}, R^{6b}, R^{6c}, R^{6d}, R^{6e}, and R^{6f} are selected such that each Si atom is substituted with no more than one optionally substituted C₂-C₆ alkenyl;
R^{6g}, R^{6h}, and R⁶ⁱ are each independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
t is an integer ranging from 300 to 2000; and
u is an integer ranging from 0 to 50.

33. The MRC of any one of claims 30-32, wherein the at least one polysiloxane (h) is a compound of formula (Ih): 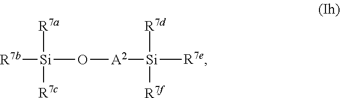
wherein:
A² comprises v units of 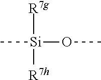
monomer and w units of 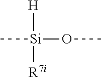
monomer,
wherein each ---- bond is a Si—O bond;
R^{7a}, R^{7b}, R^{7c}, R^{7d}, R^{7e}, and R^{7f} are each independently selected from the group consisting of H, optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl,
wherein R^{7a}, R^{7b}, R^{7c}, R^{7d}, R^{7e}, and R^{7f} are selected such that each Si atom is substituted with no more than one H atom;
each occurrence of R^{7g}, R^{7h}, and R⁷ⁱ is independently selected from the group consisting of optionally substituted C₁-C₆ alkyl, optionally substituted C₃-C₈ cycloalkyl, optionally substituted C₂-C₆ heteroalkyl, optionally substituted C₁-C₈ heterocycloalkyl, optionally substituted benzyl, optionally substituted C₆-C₁₀ aryl, and optionally substituted C₂-C₁₂ heteroaryl;
v is an integer ranging from 10 to 500; and
w is an integer ranging from 2 to 10.

34. The MRC of any one of claims 30-33, wherein at least one of the following applies:
(d) R^{6a}, R^{6b}, R^{6c}, R^{6d}, R^{6e}, and R^{6f} are each independently selected from the group consisting of CH₃ and —CH═CH₂;
(e) R^{6g}, R^{6h}, and R⁶ⁱ are each independently CH₃;
(c) R^{7a}, R^{7b}, R^{7c}, R^{7d}, R^{7e}, and R^{7f} are each independently selected from the group H and CH₃ R^{6g}, R^{6h}, and R⁶ⁱ are each independently CH₃; and
(f) R^{7g}, R^{7h}, and R⁷ⁱ are each independently CH₃.

35. The MRC of any one of claims 30-34, wherein the silicone miscible, volatile fluid is at least one selected from the group consisting of disiloxane, trisiloxane, and decamethyl cyclopentasiloxane.

36. The MRC of any one of claims 30-35, wherein the reinforcing agent is at least one selected from the group consisting of silica and HMDS treated fumed silica.

37. The MRC of any one of claims 30-36, wherein the composition has a total ratio of units of silicon hydride (i.e., Si—H) to vinyl-substituted silicon (i.e., Si—C(H)═CH₂) in all reactant components ranging from about 1 to about 20.

38. The MRC of any one of claims 30-37, wherein the composition has a total ratio of units of silicon hydride (i.e., Si—H) to vinyl-substituted silicon (i.e., Si—C(H)═CH₂) in all reactant components ranging from about 5 to about 7.

39. The MRC of any one of claims 30-38, wherein the composition further comprises one or more additives.

40. The MRC of claim 39, wherein the additive is a rheology modifier.

41. The MRC of claim 39 or 40, wherein the additive is an aesthetic and/or cosmetic modifier.

42. The MRC of claim 41, wherein the aesthetic and/or cosmetic modifier is at least one selected from the group consisting of vitamin A, vitamin B3, vitamin C, vitamin D, vitamin E, vitamin F, vitamin K, glycolic acid, sunscreen, and/or panthenol.

43. The MRC of any one of claims 39-42, wherein the additive is a pharmaceutically active compound and/or composition.

44. The MRC of claim 43, wherein the pharmaceutically active additive is at least one selected from the group consisting of one or more steroids (e.g., mometasone, clobetasol, triamcinolone, fluocinonide, flurandrenolide, clocortolone, halobetasol, desoximetasone, desonide, hydrocortisone, betamethasone, fluticasone, halcinonide, fluocinolone, prednicarbate, diflorasone, flurandrenolide, amcinonide and alclometasone), one or more retinoids (e.g., tretinoin, adapalene, tazarotene, alitretinoin and bexarotene), benzoyl peroxide, azelaic acid, diamino-diphenyl sulphone, one or more JAK inhibitors (e.g., ruxolitinib and delgocitinib), one or more antibiotics (e.g., fusidic acid, mupirocin, retapamulin, silver sulfadiazine, bacitracin, neomycin, polymyxin B, sulfacetamide sodium, sulfur, ozenoxacin, silver sulfadiazine, erythromycin, mafenide, gentamicin, clindamycin, metronidazole, gentamicin, and nadifloxacin), one or more calcineurin inhibitors (e.g., tacrolimus and pimecrolimus), one or more antifungals (e.g., clotrimazole, terbinafine, miconazole, econazole, ketoconazole, tioconazole and amorolfine), becaplermin, 5-fluorouracil, diclofenac, and imiquimod.

45. The MRC of any one of claims 35-44, wherein at least one of the following applies:
(a) the at least one polysiloxane (g) comprises vinyl terminated dimethylpolysiloxane;
(b) the at least one polysiloxane (h) comprises trimethylsiloxy terminated, pendant silicon-hydride functional poly dimethylsiloxane;
(c) the at least one reinforcing material comprises HMDS treated fumed silica; and
(d) the at least one silicone miscible, volatile fluid comprises decamethyl cyclopentasiloxane.

46. The MRC of claim 45, wherein at least one of the following applies:
(a) the at least one polysiloxane (g) comprises about 30% to about 50% of the composition by weight (w/w %);
(b) the at least one polysiloxane (h) comprises about 1% to about 10% of the composition by weight (w/w %);
(c) the at least one reinforcing material comprises about 10% to about 30% of the composition by weight (w/w %); and
(d) the at least one silicone miscible, volatile fluid comprises about 35% to about 50% of the composition by weight (w/w %).

47. The MRC of claim 45 or 46, wherein at least one of the following applies:
(a) the at least one polysiloxane (g) comprises about 36.6% of the composition by weight (w/w %);
(b) the at least one polysiloxane (h) comprises about 6.2% of the composition by weight (w/w %);
(c) the at least one reinforcing material comprises about 14.5% of the composition by weight (w/w %); and
(d) the at least one silicone miscible, volatile fluid comprises about 42.7% of the composition by weight (w/w %).

48. The MRC of any one of claims 30-44, wherein at least one of the following applies:
(a) the at least one polysiloxane (g) comprises vinyl terminated dimethylpolysiloxane;
(b) the at least one polysiloxane (h) comprises trimethylsiloxy terminated, pendant silicon-hydride functional poly dimethylsiloxane;
(c) the at least one reinforcing material comprises silica and HMDS treated fumed silica;
(d) the at least one silicone miscible, volatile fluid comprises decamethyl cyclopentasiloxane; and
(e) the at least one non-volatile silicone miscible fluid comprises polydimethylsiloxane fluid.

49. The MRC of any one of claims 30-44 and 48, wherein at least one of the following applies:
(a) the at least one polysiloxane (g) comprises about 20% to about 40% of the composition by weight (w/w %);
(b) the at least one polysiloxane (h) comprises about 1% to about 10% of the composition by weight (w/w %);
(c) the at least one reinforcing material comprises about 10% to about 30% of the composition by weight (w/w %);
(d) the at least one silicone miscible, volatile fluid comprises about 35% to about 50% of the composition by weight (w/w %); and
(e) the at least one non-volatile silicone miscible fluid comprises about 0.1 to about 5% of the composition by weight (w/w %).

50. The MRC of claim 49, wherein at least one of the following applies:
(a) the at least one polysiloxane (g) comprises about 34.4%, 34.8%, or about 36.2% of the composition by weight (w/w %);
(b) the at least one polysiloxane (h) comprises about 5.8%, 5.9%, or about 6.1% of the composition by weight (w/w %);
(c) the at least one reinforcing material comprises two components which in total comprise about 14.4%, 18.6%, or about 18.8% of the composition by weight (w/w %);
(d) the at least one silicone miscible, volatile fluid comprises about 40.1%, 40.6%, or about 42.3% of the composition by weight (w/w %); and
(e) the at least one non-volatile silicone miscible fluid comprises about 0% or about 1% of the composition by weight (w/w %).

51. The MRC of any one of claims 30-44 and 48-50, wherein at least one of the following applies:
(a) the at least one polysiloxane (g) comprises about 30% of the composition by weight (w/w %);
(b) the at least one polysiloxane (h) comprises about 5% of the composition by weight (w/w %);
(c) the at least one reinforcing material comprises about 22% of the composition by weight (w/w %);
(d) the at least one silicone miscible, volatile fluid comprises about 42% of the composition by weight (w/w %); and
(e) the at least one non-volatile silicone miscible fluid comprises about 0.7% of the composition by weight (w/w %).

52. The MRC of any one of claims 30-44 and 48-51, wherein at least one of the following applies:
(a) the at least one polysiloxane (g) comprises two vinyl terminated dimethylpolysiloxanes;
(b) the at least one polysiloxane (h) comprises two trimethylsiloxy terminated, pendant silicon-hydride functional polydimethylsiloxanes;
(c) the at least one reinforcing material comprises silica and HMDS treated fumed silica; and
(d) the at least one silicone miscible, volatile fluid comprises decamethyl cyclopentasiloxane.

53. The MRC of any one of claims 30-44 and 52, wherein at least one of the following applies:
(a) the at least one polysiloxane (g) comprises about 20% to about 40% of the composition by weight (w/w %);
(b) the at least one polysiloxane (h) comprises about 1% to about 10% of the composition by weight (w/w %);
(c) the at least one reinforcing material comprises about 5% to about 25% of the composition by weight (w/w %); and
(d) the at least one silicone miscible, volatile fluid comprises about 40% to about 60% of the composition by weight (w/w %).

54. The MRC of any one of claims 30-44 and 52-53, wherein at least one of the following applies:
(a) the at least one polysiloxane (g) comprises about 27% of the composition by weight (w/w %);
(b) the at least one polysiloxane (h) comprises about 6% of the composition by weight (w/w %);
(c) the at least one reinforcing material comprises about 13% of the composition by weight (w/w %); and
(d) the at least one silicone miscible, volatile fluid comprises about 54% of the composition by weight (w/w %).

55. A multilayer composition comprising:
(a) an adhesive basal layer comprising the composition of any one of claims 1-26, wherein the adhesive basal layer optionally further comprises one or more additional compounds and/or compositions selected from the group consisting of a non-reactive fluid, emulsifier, a cosmetic, and a pharmaceutically active agent and/or composition;
(b) a mechanically reinforcing layer comprising the MRC of any one of claims 30-54;
wherein the adhesive basal layer is in contiguous contact with at least a portion of a surface of an object; and
wherein the mechanically reinforcing layer is in contiguous contact with at least a portion of the surface of the adhesive basal layer.

56. A method for applying a multilayered wound dressing composition to a wound of a subject, the method comprising:
(a) applying to the surface of the wound an adhesive basal layer comprising the composition of any one of claims 1-26, wherein the adhesive basal layer optionally further comprises one or more additional compounds and/or compositions selected from the group consisting of a non-reactive fluid, emulsifier, and a pharmaceutically active agent and/or composition; and
(b) applying to the surface of the adhesive basal layer a mechanically reinforcing layer comprising the MRC of any one of claims 30-54;
wherein the adhesive basal layer is in contiguous contact with at least a portion of the surface of the wound; and
wherein the mechanically reinforcing layer is in contiguous contact with at least a portion of the surface of the adhesive basal layer.

57. A method of treating a skin condition and/or wound of a subject, the method comprising:
(a) applying to the surface of the wound an adhesive basal layer comprising the composition of any one of claims 1-26, wherein the adhesive basal layer optionally further comprises one or more additional compounds and/or compositions selected from the group consisting of a non-reactive fluid, emulsifier, a cosmetic, and a pharmaceutically active agent and/or composition; and
(b) applying to the surface of the adhesive basal layer a mechanically reinforcing layer comprising the MRC of any one of claims 30-54;
wherein the adhesive basal layer is in contiguous contact with at least a portion of the surface of the wound; and
wherein the mechanically reinforcing layer is in contiguous contact with at least a portion of the surface of the adhesive basal layer.

58. A kit comprising:
(a) a container comprising the composition of any one of claims 1-26, wherein the container is suitable for dispensation;
(b) a container comprising the MRC of any one of claims 30-54, wherein the container is suitable for dispensation; and
(c) instructional materials for use thereof.

59. An emulsion composition comprising:
(a) at least one cross-linked telechelic polymer composition,
wherein the at least one cross-linked telechelic polymer composition comprises about 1% to about 25% of the emulsion composition (w/w %);
(b) at least one emulsifier,
wherein the at least one emulsifier comprises about 0.10% to about 10% of the emulsion composition (w/w %);
(c) at least one polar solvent or water-miscible solvent,
wherein the at least one polar solvent or water-miscible solvent comprises about 50% to about 99% of the emulsion composition (v/w %); and
(d) at least one silicone fluid,
wherein the at least one silicone fluid comprises about 1% to about 25% of the emulsion composition (w/w %).

60. The emulsion of claim 59, wherein at least one of the following applies:
(a) the at least one cross-linked telechelic polymer composition is the composition of any one of claims 1-26;
(b) the at least one cross-linked telechelic polymer composition comprises about 10% of the emulsion composition (w/w %);
(c) the at least one emulsifier comprises cetyl diglyceryl tris(trimethylsiloxy)silylethyl dimethicone;
(d) the at least one emulsifier comprises about 1% of the emulsion composition (w/w %);
(e) the at least one polar solvent or water-miscible solvent comprises at least one selected from the group consisting of 1,3-butylene glycol and glycerin;
(f) the at least one polar solvent or water-miscible solvent comprises about 75% of the emulsion composition (w/w %);
(g) the at least one silicone fluid comprises at least one selected from the group consisting of caprylyl methicone and dimethicone; and
(h) the at least one silicone fluid comprises about 14% of the emulsion composition (w/w %).
